(19) United States
(12) Patent Application Publication
(10) Pub. No.: US 2009/0069373 A1
(43) Pub. Date: Mar. 12, 2009

(54) Quinoline Acids

(75) Inventors: Jay E. Wrobel, Lawrenceville, NJ (US); Baihua Hu, Audubon, PA (US); Michael David Collini, Clifton, PA (US); James Winfield Jetter, Norristown, PA (US); Ronald Charles Bernotas, Wayne, PA (US); David Harry Kaufman, Schwenksville, PA (US); Robert Ray Singhaus, JR., Pottstown, PA (US); John William Ullrich, Downingtown, PA (US); Robert Lester Morris, Wayne, PA (US); Rayomand J. Unwalla, Eagleville, PA (US)

Correspondence Address: FISH & RICHARDSON P.C., P.O BOX 1022, MINNEAPOLIS, MN, 55440-1022, US

(73) Assignee: Wyeth, Madison, NJ (US)

(21) Appl. No.: 12/039,347

(22) Filed: Feb. 28, 2008

Related U.S. Application Data
(60) Provisional application No. 60/903,942, filed on Feb. 28, 2007.

(51) Int. Cl. A61K 31/47; C07D 215/12; C07D 401/02; A61P 9/00; A61P 25/28; A61K 31/4709; A61P 3/00; A61P 29/00; A61P 17/00

(52) U.S. Cl. 514/313; 546/152; 546/167; 514/311

(57) ABSTRACT

This invention relates generally to quinoline-based modulators of Liver X receptors (LXRs) and related methods.

### CROSS REFERENCE TO RELATED APPLICATIONS

This application claims the benefit of U.S. Provisional Application No. 60/903,942, filed on Feb. 28, 2007, which is incorporated herein by reference in its entirety.

### TECHNICAL FIELD

This invention relates generally to quinoline-based modulators of Liver X receptors (LXRs) and related methods.

### BACKGROUND

Atherosclerosis is among the leading causes of death in developed countries. Some of the independent risk factors associated with atherosclerosis include the presence of relatively high levels of serum LDL cholesterol and relatively low levels of serum HDL cholesterol in affected patients. As such, some anti-atherosclerotic therapy regimens include the administration of agents (e.g., statins) to reduce elevated serum LDL cholesterol levels.

Agents that increase patient HDL cholesterol levels can also be useful in anti-atherosclerotic therapy regimens. HDL cholesterol is believed to play a major role in the transport of cholesterol from peripheral tissues to the liver for metabolism and excretion (this process is sometimes referred to as “reverse cholesterol transport”). ABCA1 is a transporter gene involved in HDL production and reverse cholesterol transport. Upregulation of ABCA1 can therefore result in increased reverse cholesterol transport as well as inhibition of cholesterol absorption in the gut. In addition, HDL is also believed to inhibit the oxidation of LDL cholesterol, reduce the inflammatory response of endothelial cells, inhibit the coagulation pathway, and promote the availability of nitric oxide.

Liver X receptors (LXRs), originally identified in the liver as orphan receptors, are members of the nuclear hormone receptor super family and are believed to be involved in the regulation of cholesterol and lipid metabolism. LXRs are ligand-activated transcription factors and bind to DNA as obligate heterodimers with retinoid X receptors. While LXRα is generally found in tissues such as liver, kidney, adipose tissue, intestine and macrophages, LXRβ displays a ubiquitous tissue distribution pattern. Activation of LXRs by oxysterols (endogenous ligands) in macrophages results in the expression of several genes involved in lipid metabolism and reverse cholesterol transport including the aforementioned ABCA1, ABCG1, and ApoE.

Studies have been conducted in LXRα knock-out (k/o), LXRβ k/o and double k/o mice to determine the physiological role of LXRs in lipid homeostasis and atherosclerosis. The data from these studies suggested that in double k/o mice on normal chow diet, increased cholesterol accumulation was observed in macrophages (foam cells) of the spleen, lung and arterial wall. The increased cholesterol accumulation was believed to be associated with the presence of reduced serum HDL cholesterol and increased LDL cholesterol, even though the total cholesterol levels in the mice were about normal. While LXRα k/o mice did not appear to show significant changes in hepatic gene expression, LXRβ k/o mice showed 58% decrease in hepatic ABCA1 expression and 208% increase in SREBP1c expression suggesting that LXRβ may be involved in the regulation of liver SREBP1c expression.

Data obtained from studies employing two different atherosclerotic mouse models (ApoE k/o and LDLR k/o) suggest that agonists of LXRα or β can be relatively effective in upregulating ABCA1 expression in macrophages. For example, inhibition of atherosclerotic lesions could be observed when ApoE k/o and LDLR k/o mice were treated with LXRα or β agonists for 12 weeks. The tested agonists were observed to have variable effects on serum cholesterol and lipoprotein levels and appeared to cause a relatively significant increase in serum HDL cholesterol and triglyceride levels. These in vivo data were found to be consistent with in vitro data obtained for the same agonists in macrophages.

In addition to the lipid and triglyceride effects described above, it is also believed that activation of LXRs results in the inhibition of inflammation and proinflammatory gene expression. This hypothesis is based on data obtained from studies employing three different models of inflammation (LPS-induced sepsis, acute contact dermatitis of the ear and chronic atherosclerotic inflammation of the artery wall). These data suggest that LXR modulators can mediate both the removal of cholesterol from the macrophages and the inhibition of vascular inflammation.

### SUMMARY

This invention relates generally to quinoline-based modulators of LXRs and related methods and compositions.

In one aspect, this invention features a compound of formula (I):

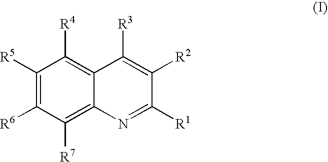

wherein:

R¹ is hydrogen or C₁-C₆ alkyl;

R² is:

(i) hydrogen; or

(ii) C₁-C₁₂ alkyl or C₁-C₁₂ haloalkyl, each of which is optionally substituted with from 1-5 R^{a}; or

(iii) C₇-C₂₀ aralkyl or heteroaralkyl including 6-20 atoms, each of which is optionally substituted with from 1-10 R^{b}; or

(iv) C₂-C₁₂ alkenyl or C₂-C₁₂ alkynyl, each of which is optionally substituted with from 1-10 R^{c};

(v) C₃-C₁₀ cycloalkyl or heterocyclyl including 3-10 atoms, each of which is optionally substituted with from 1-5 R^{b}; or

(vi) C₆-C₁₈ aryl or heteroaryl including 5-16 atoms, each of which is optionally substituted with from 1-10 R^{d};

R³ is C₆-C₁₈ aryl, heteroaryl including 5-16 atoms, C₈-C₂₀ arylcycloalkyl, heteroarylcycloalkyl including 8-20 atoms, C₈-C₂₀ arylcycloalkenyl, heteroarylcycloalkenyl including 8-20 atoms, arylheterocyclyl including 8-20 atoms, heteroarylheterocyclyl including 8-20 atoms, arylheterocycloalkenyl including 8-20 atoms, heteroarylheterocycloalkenyl including 8-20 atoms, each of which is:

(i) substituted with from 1-5 R⁸, and

(ii) optionally substituted with from 1-4 R^{e}; wherein:

R⁸ is WA, wherein:

W at each occurrence is, independently, a bond; —O—; —S(O)ₜ—, wherein t is 0-2; —NR⁹—, wherein R⁹ is hydrogen or C₁-C₆ alkyl; C₁₋₆ alkylene; C₂₋₆ alkenylene; C₂₋₆ alkynylene; C₃₋₆ cycloalkylene; —W¹(C₁₋₆ alkylene)-; or —(C₁₋₆ alkylene)W¹—;

W¹ at each occurrence is, independently, —O—; —S(O)ₜ—, wherein t is 0-2; or —NR⁹—, wherein R⁹ is hydrogen or C₁-C₆ alkyl; and

A at each occurrence is, independently, C₆-C₁₈ aryl, heteroaryl including 5-16 atoms, C₈-C₂₀ arylcycloalkyl, heteroarylcycloalkyl including 8-20 atoms, C₈-C₂₀ arylcycloalkenyl, heteroarylcycloalkenyl including 8-20 atoms, arylheterocyclyl including 8-20 atoms, heteroarylheterocyclyl including 8-20 atoms, arylheterocycloalkenyl including 8-20 atoms, heteroarylheterocycloalkenyl including 8-20 atoms, each of which is:

(i) substituted with from 1-5 R¹⁰, and

(ii) optionally substituted with from 1-10 (e.g., 1-8, 1-6, 1-5, 1-4, 1-3, 1-2, 1, e.g., 1-6, 1-4) R^{e}; wherein:

R¹⁰ at each occurrence is, independently:

(i) —W²—C(O)OR¹¹; or

(ii)

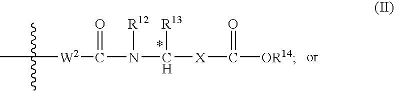

(iii) —W²—C(O)NR¹⁵R¹⁶; wherein:

W² at each occurrence is, independently, a bond; C₁₋₆ alkylene optionally substituted with from 1-3 R^{b}; C₂₋₆ alkenylene; C₂₋₆ alkynylene; C₃₋₆ cycloalkylene; or —W³(C₁₋₆ alkylene)-;

W³ at each occurrence is, independently, —O—; —S(O)ₜ—, wherein t is 0-2; or —NR⁹—,

wherein R⁹ is hydrogen or C₁-C₆ alkyl;

each of R¹¹, R¹³, and R¹⁴ at each occurrence is, independently:

(i) hydrogen; or

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{c}; or

(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{b}; or

(v) C₆-C₁₈ aryl or heteroaryl including 5-16 atoms, each of which is optionally substituted with from 1-10 R^{d};

R¹² is hydrogen or C₁-C₆ alkyl;

X is a bond; C₁₋₆ alkylene optionally substituted with from 1-5 substituents selected from halo or R^{a}; C₂₋₆ alkenylene or C₂₋₆ alkynylene optionally substituted with from 1-5 R^{c}; or C₃₋₆ cycloalkylene optionally substituted with from 1-5 R^{b}; and

R¹⁵ and R¹⁶ together with the nitrogen atom to which each is attached is heterocyclyl including 3-10 atoms, which is:

(i) substituted with from 1-2 R^{f}; and

(ii) optionally substituted with from 1-4 R^{e};

each of R⁴, R⁵, R⁶, and R⁷ is, independently:

(i) hydrogen; or

(ii) R^{c}; or

(iii) C₁-C₂₀ alkyl or C₁-C₂₀ haloalkyl, each of which is optionally substituted with from 1-10 R^{a}; or

(iv) C₂-C₂₀ alkenyl or C₂-C₂₀ alkynyl, each of which is optionally substituted with from 1-10 R^{c}; or

(iv) C₇-C₂₀ aralkyl or heteroaralkyl including 6-20 atoms, each of which is optionally substituted with from 1-10 R^{b};

R^{a} at each occurrence is, independently:

(i) NR^{g}R^{h}; nitro; azido; hydroxy; oxo; thioxo; ═NRⁱ; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{b}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{b}; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h}); or

(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{b};

R^{a′} at each occurrence is, independently, NR^{g}R^{h}; nitro; azido; hydroxy; oxo; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; —P(O)(OR^{g})(OR^{h}); C₃-C₂₀ cycloalkyl, C₃-C₂₀ cycloalkenyl, heterocyclyl including 3-20 atoms, or heterocycloalkenyl including 3-20 atoms;

R^{b} at each occurrence is, independently:

(i) halo; NR^{g}R^{h}; nitro; azido; hydroxy; oxo; thioxo; ═NRⁱ; C₁-C₂₀ alkoxy or C₁-C₂₀ haloalkoxy, each of which is optionally substituted with from 1-10 R^{a}; C₆-C₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{b′}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{b′}; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h}); or

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{c}; or

(iv) C₆-C₁₈ aryl or heteroaryl including 5-16 atoms, each of which is optionally substituted with from 1-10 R^{d}; or

(v) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{b′};

R^{b′} at each occurrence is, independently, R^{a′}; halo; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₁-C₂₀ alkyl or C₁-C₂₀ haloalkyl, each of which is optionally substituted with from 1-10 R^{a}; C₂-C₂₀ alkenyl; C₂-C₂₀ alkynyl; or C₆-C₁₈ aryl or heteroaryl including 5-16 atoms, each of which is optionally substituted with from 1-10 R^{d};

R^{c} at each occurrence is, independently:

(i) halo; NR^{g}R^{h}; nitro; azido; hydroxy; oxo; thioxo; ═NRⁱ; C₁-C₂₀ alkoxy or C₁-C₂₀ haloalkoxy, each of which is optionally substituted with from 1-10 R^{a}; C₆-C₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{b}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{b}; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h}); or

(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{b}; or

(iii) C₆-C₁₈ aryl or heteroaryl including 5-16 atoms, each of which is optionally substituted with from 1-10 R^{d};

R^{d} at each occurrence is, independently:

(i) halo; NR^{g}R^{h}; nitro; azido; hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d′}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{b}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d′}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{b}; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h});

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{c}; or

(iv) C₇-C₂₀ aralkyl, heteroaralkyl including 6-20 atoms, C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{b}; or

(v) C₆-C₁₈ aryl or heteroaryl including 5-16 atoms, each of which is optionally substituted with from 1-10 R^{d′};

R^{d′} at each occurrence is, independently, halo; NR^{g}R^{h}; nitro; azido; hydroxy; C₁-C₂₀ alkyl, C₁-C₂₀ haloalkyl, C₂-C₂₀ alkenyl; C₂-C₂₀ alkynyl; C₃-C₂₀ cycloalkyl; C₃-C₂₀ cycloalkenyl, heterocyclyl including 3-20 atoms; heterocycloalkenyl including 3-20 atoms; C₇-C₂₀ aralkyl; heteroaralkyl including 6-20 atoms; C₁-C₂₀ alkoxy; C₁-C₂₀ haloalkoxy; C₆-C₁₈ aryloxy; heteroaryloxy; C₇-C₂₀ aralkoxy; heteroaralkoxy including 6-20 atoms; C₃-C₁₆ cycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₇-C₂₀ thioaralkoxy, thioheteroaralkoxy including 6-20 atoms, C₃-C₁₆ thiocycloalkoxy C₃-C₂₀ thiocycloalkenyloxy, thioheterocyclyloxy including 3-20 atoms, or thioheterocycloalkenyloxy including 3-20 atoms; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h});

R^{e} at each occurrence is, independently, C₁-C₆ alkyl, optionally substituted with from 1-3 R^{a}; C₁-C₆ haloalkyl; mercapto; C₁-C₆ thioalkoxy optionally substituted with from 1-3 R^{a}; C₆-C₁₀ aryl or C₆-C₁₀ aryloxy, each of which is optionally substituted with from 1-10 R^{d}; halo; hydroxyl; NR^{g}R^{h}; nitro; C₂-C₆ alkenyl; C₂-C₆ alkynyl; C₁-C₆ alkoxy; C₁-C₆ haloalkoxy; cyano; —C(O)OR^{j}; or —C(O)R^{j};

R^{f} at each occurrence is, independently, —X—C(O)OR¹⁴, wherein each of X and R¹⁴ at each occurrence is, independently, as defined above;

each of R^{g}, R^{h}, Rⁱ, and R^{k}, at each occurrence is, independently:

(i) hydrogen; or

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{c}; or

(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{b}; or

(v) C₆-C₁₈ aryl or heteroaryl including 5-16 atoms, each of which is optionally substituted with from 1-10 R^{d}; or

(vi) —C(O)R^{j}, —C(O)OR^{j}; or —S(O)ₙR^{m};

R^{j} at each occurrence is, independently:

(i) hydrogen; or

(ii) C₁-C₂₀ alkyl or C₁-C₂₀ haloalkyl, each of which is optionally substituted with from 1-10 R^{a}; or

(iii) C₂-C₂₀ alkenyl or C₂-C₂₀ alkynyl, each of which is optionally substituted with from 1-10 R^{c}; or

(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{b}; or

(v) C₆-C₁₈ aryl or heteroaryl including 5-16 atoms, each of which is optionally substituted with from 1-10 R^{d}; and

R^{m} at each occurrence is, independently, R^{j}, OR^{j}, or NR^{g}R^{h};

or an S- or N-oxide and/or a salt (e.g., a pharmaceutically acceptable salt) thereof.

In one aspect, this invention relates to any of the specific quinoline compounds delineated herein (e.g., as shown in Examples 1-131, e.g., Examples 1-10 and 12-27).

In one aspect, this invention features a pharmaceutical composition, which includes a compound of formula (I) (including any subgenera or specific compounds thereof) or a salt (e.g., a pharmaceutically acceptable salt) or a prodrug thereof and a pharmaceutically acceptable adjuvant, carrier or diluent. In some embodiments, the composition can include an effective amount of the compound or the salt thereof. In some embodiments, the composition can further include an additional therapeutic agent.

The invention also relates generally to modulating (e.g., activating) LXRs with the quinoline compounds described herein. In some embodiments, the methods can include, e.g., contacting an LXR in a sample (e.g., a tissue, a cell free assay medium, a cell-based assay medium) with a compound of formula (I) (including any subgenera or specific compounds thereof). In other embodiments, the methods can include administering a compound of formula (I) (including any subgenera or specific compounds thereof) to a subject (e.g., a mammal, e.g., a human, e.g., a human having or at risk of having one or more of the diseases or disorders described herein).

In one aspect, this invention features a method of selectively modulating (e.g., activating) LXRβ (e.g., selectively modulating LXRβ relative to LXRα, e.g., selectively activating LXRβ relative to LXRα). In some embodiments, a compound of formula (I) can have an LXRα/LXRβ binding ratio of from about 1.5 to about 1,000 (e.g., from about 1.5 to about 500, from about 1.5 to about 100, from about 2.0 to about 100, from about 2.0 to about 50, from about 9 to about 50, from about 10 to about 50, from about 20 to about 50, from about 30 to about 50, from about 40 to about 50).

As used herein, the term “LXRα/LXRβ binding ratio” refers to the following ratio: IC50 (μM) LXRα binding/IC50 (μM) LXRβ binding.

In certain embodiments, a compound of formula (I) can have an LXRα/LXRβ binding ratio of from about 9 to about 19; from about 20 to about 27; from about 28 to about 44 (e.g., from about 28 to about 38); or from about 45 to about 50.

In one aspect, this invention also relates generally to methods of treating (e.g., controlling, ameliorating, preventing, delaying the onset of, or reducing the risk of developing) one or more LXR-mediated diseases or disorders in a subject (e.g., a subject in need thereof). The methods include administering to the subject an effective amount of a compound of formula (I) (including any subgenera or specific compounds thereof) or a pharmaceutically acceptable salt or prodrug thereof. LXR-mediated diseases or disorders can include, e.g., cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging or connective tissue diseases.

In another aspect, this invention relates to methods of modulating (e.g., increasing) serum HDL cholesterol levels in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., decreasing) serum LDL cholesterol levels in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., decreasing) serum LDL cholesterol levels in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., increasing) reverse cholesterol transport in a subject (e.g., a subject in need thereof), which includes administering to the subject an effective amount of a compound of formula (I) (including any subgenera or specific compounds thereof) or a pharmaceutically acceptable salt or prodrug thereof.

In another aspect, this invention relates to methods of modulating (e.g., decreasing or inhibiting) cholesterol absorption in a subject (e.g., a subject in need thereof), which includes administering to the subject an effective amount of a compound of formula (I) (including any subgenera or specific compounds thereof) or a pharmaceutically acceptable salt or prodrug thereof.

In a further aspect, this invention relates to methods of preventing or treating a cardiovascular disease (e.g., acute coronary syndrome, restenosis), which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating a atherosclerosis and/or atherosclerotic lesions, which includes administering to a subject in need thereof an effective amount of a compound of formula (I) (including any subgenera or specific compounds thereof) or a pharmaceutically acceptable salt or prodrug thereof.

In another aspect, this invention relates to methods of preventing or treating diabetes (e.g., type I diabetes or type 2 diabetes), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating Syndrome X, which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating a obesity, which includes administering to a subject in need thereof an effective amount of a compound of formula (I) (including any subgenera or specific compounds thereof) or a pharmaceutically acceptable salt or prodrug thereof.

In another aspect, this invention relates to methods of preventing or treating a lipid disorder (e.g., dyslipidemia, hyperlipidemia, hypertriglyceridemia, hypercholesterolemia, low HDL and high LDL), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating a cognitive disorder (e.g., Alzheimer's disease or dementia), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating a Alzheimer's disease or dementia, which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating a Alzheimer's disease, which includes administering to a subject in need thereof an effective amount of a compound of formula (I) (including any subgenera or specific compounds thereof) or a pharmaceutically acceptable salt or prodrug thereof.

In one aspect, this invention relates to methods of preventing or treating an inflammatory disease (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating celiac, which includes administering to a subject in need thereof an effective amount of a compound of formula (I) (including any subgenera or specific compounds thereof) or a pharmaceutically acceptable salt or prodrug thereof.

In a further aspect, this invention relates to methods of preventing or treating thyroiditis, which includes administering to a subject in need thereof an effective amount of a compound of formula (I) (including any subgenera or specific compounds thereof) or a pharmaceutically acceptable salt or prodrug thereof.

In one aspect, this invention relates to methods of treating a connective tissue disease (e.g., osteoarthritis or tendonitis), which includes administering to a subject (e.g., a mammal, e.g., a human) in need thereof an effective amount of a compound of formula (I) (including any subgenera or specific compounds thereof) or a pharmaceutically acceptable salt or prodrug thereof. In embodiments, the compound of formula (I) inhibits (e.g., reduces or otherwise diminishes) cartilage degradation. In embodiments, the compound of formula (I) induces (e.g., increases or otherwise agments) cartilage regeneration. In embodiments, the compound of formula (I) inhibits (e.g., reduces or otherwise diminishes) cartilage degradation and induces (e.g., increases or otherwise agments) cartilage regeneration. In embodiments, the compound of formula (I) inhibits (e.g., reduces or otherwise diminishes) aggrecanase activity. In embodiments, the compound of formula (I) inhibits (e.g., reduces or otherwise diminishes) elaboration of pro-inflammatory cytokines in osteoarthritic lesions.

In another aspect, this invention relates to methods of treating or preventing skin aging, the method comprising administering (e.g., topically administering) to a subject (e.g., a mammal, e.g., a human) in need thereof an effective amount of a compound of formula (I) (including any subgenera or specific compounds thereof) or a pharmaceutically acceptable salt or prodrug thereof. In embodiments, the skin aging can be derived from chronological aging, photoaging, steroid-induced skin thinning, or a combination thereof.

The term “skin aging” includes conditions derived from intrinsic chronological aging (for example, deepened expression lines, reduction of skin thickness, inelasticity, and/or unblemished smooth surface), those derived from photoaging (for example, deep wrinkles, yellow and leathery surface, hardening of the skin, elastosis, roughness, dyspigmentations (age spots) and/or blotchy skin), and those derived from steroid-induced skin thinning. Accordingly, another aspect is a method of counteracting UV photodamage, which includes contacting a skin cell exposed to UV light with an effective amount of a compound of formula (I).

In some embodiments, the compound of formula (I) (including any subgenera or specific compounds thereof) does not substantially increase serum and/or hepatic triglyceride levels of the subject.

In some embodiments, the administered compound of formula (I) (including any subgenera or specific compounds thereof) can be an LXR agonist (e.g., an LXRα agonist or an LXRβ agonist, e.g., an LXRβ agonist).

In some embodiments, the subject can be a subject in need thereof (e.g., a subject identified as being in need of such treatment). Identifying a subject in need of such treatment can be in the judgment of a subject or a health care professional and can be subjective (e.g. opinion) or objective (e.g. measurable by a test or diagnostic method). In some embodiments, the subject can be a mammal. In certain embodiments, the subject is a human.

In a further aspect, this invention also relates to methods of making compounds described herein. Alternatively, the method includes taking any one of the intermediate compounds described herein and reacting it with one or more chemical reagents in one or more steps to produce a compound described herein.

In one aspect, this invention relates to a packaged product. The packaged product includes a container, one of the aforementioned compounds in the container, and a legend (e.g., a label or an insert) associated with the container and indicating administration of the compound for treatment and control of the diseases or disorders described herein.

Embodiments can include one or more of the following features.

In some embodiments, when:

(i) R³ is phenyl that is mono-substituted at the meta position with 1 R⁸ (i.e., meta relative to the ring atom that serves as the point of connection of the phenyl ring to the 4-position of the quinoline ring in formula (I)), and each of the remaining ring atoms on the phenyl ring are attached to hydrogen; and

(ii) W is —OCH₂—, —NHCH₂—, —CH₂NH—, or —N(CH₃)CH₂—; and

(iii) A is phenyl that is mono-substituted at the para position with 1 CH₂C(O)OH (i.e., para relative to the ring atom that serves as the point of connection of the phenyl ring to variable W), and each of the remaining ring atoms on the phenyl ring are attached to hydrogen;

then R² cannot be C₇-C₁₂ (e.g., C₇-C₁₀, C₇) aralkyl (e.g., R² cannot be benzyl under these conditions).

In certain embodiments, the compound of formula (I) cannot be:
2-(4-((3-(3-benzyl-8-(trifluoromethyl)quinolin-4-yl)phenoxy)methyl)phenyl)acetic acid;
2-(4-((3-(3-benzyl-8-(trifluoromethyl)quinolin-4-yl)phenylamino)methyl)phenyl)acetic acid;
2-(4-(3-(3-benzyl-8-(trifluoromethyl)quinolin-4-yl)benzylamino)phenyl)acetic acid; and
2-(4-(((3-(3-benzyl-8-(trifluoromethyl)quinolin-4-yl)phenyl)(methyl)amino)methyl)phenyl)acetic acid,

each of which are described, e.g., in Hu, et al., J. Med. Chem. 2006, 49, 6151-6154 as compound 15, 16, 17, and 18, respectively.

In some embodiments, when A is C₆-C₁₈ (e.g., C₆-C₁₄, C₆-C₁₀, C₆) aryl, then A must be further substituted with from 1-10 (e.g., 1-8, 1-6, 1-5, 1-4, 1-3, 1-2, 1, e.g., 1-6, 1-4) R^{e} (i.e., substituted with one or more R^{e} in addition to being substituted with one or more R¹⁰). For example, when A is phenyl under these conditions, the phenyl ring must be further substituted with 1-4 (e.g., 1-3, 1-2, 1) R^{e} in addition to being substituted with one or more R¹⁰.

In some embodiments, when A is C₆-C₁₈ (e.g., C₆-C₁₄, C₆-C₁₀, C₆) aryl and is substituted with 1 R¹⁰, and R¹⁰ is W²—C(O)OR¹¹, then A must be further substituted with from 1-10 (e.g., 1-8, 1-6, 1-5, 1-4, 1-3, 1-2, 1, e.g., 1-6, 1-4) R^{e} (i.e., substituted with one or more R^{e} in addition to being substituted with W²—C(O)OR¹¹). For example, when A is phenyl under these conditions, the phenyl ring must be further substituted with 1-4 (e.g., 1-3, 1-2, 1) R^{e} in addition to being substituted with W²—C(O)OR¹¹.

In some embodiments, when A is C₆-C₁₈ (e.g., C₆-C₁₄, C₆-C₁₀, C₆) aryl and is substituted with 1 R¹⁰, and R¹⁰ is W²—C(O)OH, and W² is C₁-C₆ (e.g., C₁-C₃, C₁) alkylene, then A must be further substituted with from 1-10 (e.g., 1-8, 1-6, 1-5, 1-4, 1-3, 1-2, 1, e.g., 1-6, 1-4) R^{e} (i.e., substituted with one or more R^{e} in addition to being substituted with W²—C(O)OH). For example, when A is phenyl under these conditions, the phenyl ring must be further substituted with 1-4 (e.g., 1-3, 1-2, 1) R^{e} in addition to being substituted with W²—C(O)OH.

In some embodiments, R¹ can be hydrogen.

In some embodiments, R² can be C₁-C₆ alkyl, which is optionally substituted with from 1-2 R^{a}. In certain embodiment, R² can be an unsubstituted C₁-C₆ alkyl, e.g., CH₃.

In some embodiments, R² can be C₇-C₁₀ aralkyl, which is optionally substituted with from 1-3 R^{b}. In certain embodiments, R² can be benzyl.

In some embodiments, R² can be hydrogen.

In some embodiments, R³ can be C₆-C₁₀ aryl, which is (a) substituted with from 1-2 (e.g., 1) R⁸; and (b) optionally substituted with from 1-2 R^{e}. In certain embodiments, R³ can be phenyl, which is (a) substituted with 1 R⁸; and (b) optionally substituted with from 1-2 R^{e}. In some embodiments, R³ can be phenyl, which is substituted with 1 R⁸.

In some embodiments, R³ can have formula (A-2):

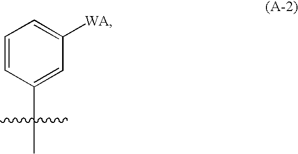

in which W and A can be as defined anywhere herein.

In some embodiments, R³ can be heteroaryl including 5-10 atoms, which is (a) substituted with 1 R⁸; and (b) optionally substituted with from 1-2 R^{e}. In certain embodiments, R³ can be pyridyl, thienyl, or indolyl, each of which is substituted with 1 R⁸.

In some embodiments, W can be —(C₁₋₆ alkylene)W¹—. In embodiments, W¹ can be —NR⁹—. In certain embodiments, R⁹ can be hydrogen. For example, W can be —(C₁₋₃ alkylene)NH—, such as —CH₂NH—.

In some embodiments, W¹ can be —O—. In some embodiments, W can be —(C₁₋₃ alkylene)O—. For example, W can be —CH₂O—.

In some embodiments, W can be —W¹(C₁₋₆ alkylene)-. In embodiments, W¹ can be —O—. In certain embodiments, W can be —O(C₁₋₃ alkylene)-. For example, W can be —OCH₂—.

In some embodiments, A can be C₆-C₁₀ aryl, which is (a) substituted with from 1-2 (e.g., 1) R¹⁰; and (b) optionally substituted with from 1-6 R^{e}. In certain embodiments, A can be C₆-C₁₀ aryl, which is (a) substituted with 1 R¹⁰; and (b) optionally substituted with from 1-6 R^{e}.

In some embodiments, A can have formula (B-1):

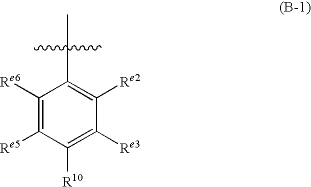

in which each of R^{e2}, R^{e3}, R^{e5}, and R^{e6} can be, independently, hydrogen or R^{e}; in which each of R^{e} and R¹⁰ can be as defined anywhere herein.

In some embodiments, A can have formula (B-2):

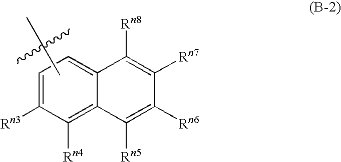

in which each of Rⁿ³ and Rⁿ⁴ can be, independently, hydrogen or R^{e}; and one of Rⁿ⁵, Rⁿ⁶, Rⁿ⁷, and Rⁿ⁸ can be R¹⁰, and the others can be each hydrogen or R^{e}; in which each of R^{e} and R¹⁰ can be as defined anywhere herein.

In some embodiments, R¹⁰ can be —W²—C(O)OR¹¹. In embodiments, R¹¹ can be hydrogen. In some embodiments, W² can be C₁-C₃ alkylene. For example, W² can be CH₂. In other embodiments, W² can be a bond. In certain embodiments, W² can be CH₂ or a bond.

In some embodiments, R¹⁰ can be:

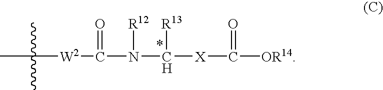

In embodiments, each of R¹² and R¹⁴ can be hydrogen, and X can be a bond. In some embodiments, R¹³ can be C₁-C₆ alkyl optionally substituted with from 1-2 R^{a} or heteroaralkyl including 6-20 atoms, each of which is optionally substituted with from 1-2 R^{b}.

In some embodiments, R¹⁰ can be —W²—C(O)NR¹⁵R¹⁶, in which R¹⁵ and R¹⁶ together with the nitrogen atom to which each is attached can be piperidin-1-yl or pyrrolidin-1-yl, which is: (i) substituted with from 1 R^{f}; and (ii) optionally substituted with from 1-2 R^{e}.

In some embodiments, R^{f} can be —X—C(O)OH. In certain embodiments, R^{f} can be —C(O)OH.

In some embodiments, each of R⁴, R⁵ and R⁶ can be hydrogen. In other embodiments, each of R⁴, R⁵ and R⁶ can be, independently, hydrogen, halo, or CH₃.

In some embodiments, R⁷ can be C₁-C₆ haloalkyl. For example, R⁷ can be CF₃.

In some embodiments, R⁷ can be halo. In certain embodiments, R⁷ can be chloro.

In some embodiments,

R² can be:

(i) hydrogen; or

(ii) C₁-C₁₂ alkyl which is optionally substituted with from 1-5 R^{a}; or

(iii) C₇-C₂₀ aralkyl, which is optionally substituted with from 1-10 R^{b}; and

R³ can be C₆-C₁₈ aryl or heteroaryl including 5-16 atoms, each of which is: (i) substituted with 1 R⁸, and (ii) optionally substituted with from 1-4 R^{e}; and

W can be —(C₁₋₆ alkylene)W¹—; and

W¹ at each occurrence can be, independently, —O— or —NR⁹—, in which R⁹ is hydrogen or C₁-C₃ alkyl; and

A at each occurrence can be, independently, C₆-C₁₈ aryl, heteroaryl including 5-16 atoms, or C₈-C₂₀ arylcycloalkenyl, each of which is: (i) substituted with 1 R¹⁰, and (ii) optionally substituted with from 1-4 R^{e}; and

R¹⁰ can be —W²—C(O)OR¹¹, and

W² can be a bond or C₁₋₆ alkylene optionally substituted with from 1-3 R^{b}.

In some embodiments, R¹, R⁴, R⁵, and R⁶ can be hydrogen, and R⁷ can be C₁-C₄ haloalkyl.

In certain embodiments, R² can be C₁-C₆ alkyl, which is optionally substituted with from 1-2 R^{a}. For example, R² can be CH₃. In certain embodiments, R² can be C₇-C₁₀ aralkyl, which is optionally substituted with from 1-3 R^{b}. For example, R² can be benzyl. In other embodiments, R² can be hydrogen. In certain embodiments, R² can be CH₃, benzyl, or hydrogen.

In certain embodiments, R³ can have formula (A-1):

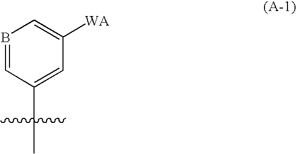

in which B can be CH or N, and each of W and A can be as defined anywhere herein. In embodiments, W can be —(C₁₋₃ alkylene)NR⁹—. In embodiments, R⁹ can be hydrogen. In some embodiments, W can be —CH₂NH—.

In certain embodiments, W can be —(C₁₋₃ alkylene)O—. For example, W can be —CH₂O—. In some embodiments, W can be —CH₂NH— or —CH₂O—.

In certain embodiments, R¹⁰ can be —W²—C(O)OH. In embodiments, W² can be a bond or C₁₋₃ alkylene. In some embodiments, R¹⁰ can be —W²—C(O)OH and W² can be a bond or C₁₋₃ alkylene.

In certain embodiments, A can have formula (B-1):

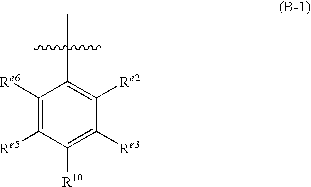

in which each of R^{e2}, R^{e3}, R^{e5}, and R^{e6} can be, independently, hydrogen, or R^{e}. In some embodiments, one or two of R^{e2}, R^{e3}, R^{e5}, and R^{e6} can each be, independently, C₁-C₄ alkyl, and the others can be hydrogen. In certain embodiments, two of R^{e2}, R^{e3}, R^{e5}, and R^{e6} can be CH₃, and the others can be hydrogen. In some embodiments, R¹⁰ can be —W²—C(O)OH. In embodiments, W² can be C₁₋₃ alkylene. For example, W² can be CH₂. In other embodiments, W² can be a bond. In some embodiments, W² can be CH₂ or a bond. In certain embodiments, two of R^{e2}, R^{e3}, R^{e5}, and R^{e6} can be CH₃, and the others can be hydrogen, R¹⁰ can be —W²—C(O)OH, and W² can be CH₂ or a bond.

In certain embodiments, A can have formula (B-2):

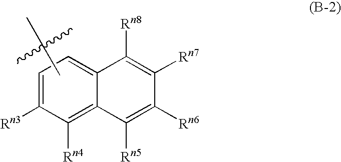

in which each of Rⁿ³, Rⁿ⁴, Rⁿ⁷, and Rⁿ⁸ can be, independently, hydrogen or R^{e}; and one of Rⁿ⁵ and Rⁿ⁶ can be R¹⁰, and the other can be hydrogen. In some embodiments, A can have formula (B-3):

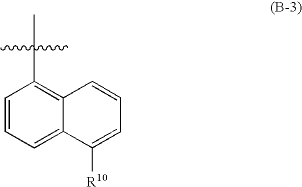

in which R¹⁰ can be —W²—C(O)OH. In embodiments, W² can be C₁₋₃ alkylene. For example, W² can be CH₂. In some embodiments, R¹⁰ can be —CH₂—C(O)OH.

In some embodiments, R⁷ can be CF₃.

The term “mammal” includes organisms, which include mice, rats, cows, sheep, pigs, rabbits, goats, and horses, monkeys, dogs, cats, and preferably humans.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect). An effective amount of the compound described above may range from about 0.01 mg/Kg to about 1000 mg/Kg, (e.g., from about 0.1 mg/Kg to about 100 mg/Kg, from about 1 mg/Kg to about 100 mg/Kg). Effective doses will also vary depending on route of administration, as well as the possibility of co-usage with other agents.

The term “halo” or “halogen” refers to any radical of fluorine, chlorine, bromine or iodine.

In general, and unless otherwise indicated, substituent (radical) prefix names are derived from the parent hydride by either (i) replacing the “ane” in the parent hydride with the suffixes “yl,” “diyl,” “triyl,” “tetrayl,” etc.; or (ii) replacing the “e” in the parent hydride with the suffixes “yl,” “diyl,” “triyl,” “tetrayl,” etc. (here the atom(s) with the free valence, when specified, is (are) given numbers as low as is consistent with any established numbering of the parent hydride). Accepted contracted names, e.g., adamantyl, naphthyl, anthryl, phenanthryl, furyl, pyridyl, isoquinolyl, quinolyl, and piperidyl, and trivial names, e.g., vinyl, allyl, phenyl, and thienyl are also used herein throughout. Conventional numbering/lettering systems are also adhered to for substituent numbering and the nomenclature of fused, bicyclic, tricyclic, polycyclic rings.

The term “alkyl” refers to a saturated hydrocarbon chain that may be a straight chain or branched chain, containing the indicated number of carbon atoms. For example, C₁-C₂₀ alkyl indicates that the group may have from 1 to 20 (inclusive) carbon atoms in it. Any atom can be substituted. Examples of alkyl groups include without limitation methyl, ethyl, and tert-butyl.

The term “cycloalkyl” refers to saturated monocyclic, bicyclic, tricyclic, or other polycyclic hydrocarbon groups. Any atom can be substituted, e.g., by one or more substituents. A ring carbon serves as the point of attachment of a cycloalkyl group to another moiety. Cycloalkyl groups can contain fused rings. Fused rings are rings that share a common carbon atom. Cycloalkyl moieties can include, e.g., cyclopropyl, cyclobutyl, cyclopentyl, cyclohexyl, cycloheptyl, adamantyl, and norbornyl (bicycle[2.2.1]heptyl).

The terms “alkylene,” “alkenylene,” “alkynylene,” and “cycloalkylene” refer to divalent alkyl (e.g., —CH₂—), alkenyl (e.g., —CH═CH—), alkynyl (e.g., —C≡C—), cycloalkyl moieties, respectively.

The term “haloalkyl” refers to an alkyl group, in which at least one hydrogen atom is replaced by halo. In some embodiments, more than one hydrogen atom (2, 3, 4, 5, 6, 7, 8, 9, 10, 11, 12, 13, 14, 15, 16, 17, 18, 19, 20, 21, 22, 23, 24, 25, 26, etc. hydrogen atoms) on a alkyl group can be replaced by more than one halogen (e.g., 2, 3, 4, 5, 6, 7, 8, 9, 10, 11, 12, 13, 14, 15, 16, 17, 18, 19, 20, 21, 22, 23, 24, 25, 26, etc. halogen atoms). In these embodiments, the hydrogen atoms can each be replaced by the same halogen (e.g., fluoro) or the hydrogen atoms can be replaced by a combination of different halogens (e.g., fluoro and chloro). “Haloalkyl” also includes alkyl moieties in which all hydrogens have been replaced by halo (e.g., perhaloalkyl, e.g., perfluoroalkyl, such as trifluoromethyl).

The term “aralkyl” refers to an alkyl moiety in which an alkyl hydrogen atom is replaced by an aryl group. One of the carbons of the alkyl moiety serves as the point of attachment of the aralkyl group to another moiety. Aralkyl includes groups in which more than one hydrogen atom on an alkyl moiety has been replaced by an aryl group. Any ring or chain atom can be substituted e.g., by one or more substituents. Non-limiting examples of “aralkyl” include benzyl, 2-phenylethyl, 3-phenylpropyl, benzhydryl (diphenylmethyl), and trityl (triphenylmethyl) groups.

The term “heteroaralkyl” refers to an alkyl moiety in which an alkyl hydrogen atom is replaced by a heteroaryl group. One of the carbons of the alkyl moiety serves as the point of attachment of the aralkyl group to another moiety. Heteroaralkyl includes groups in which more than one hydrogen atom on an alkyl moiety has been replaced by a heteroaryl group. Any ring or chain atom can be substituted e.g., by one or more substituents. Heteroaralkyl can include, for example, 2-pyridylethyl.

The term “alkenyl” refers to a straight or branched hydrocarbon chain containing 2-20 carbon atoms and having one or more double bonds. Any atom can be substituted, e.g., by one or more substituents. Alkenyl groups can include, e.g., allyl, 1-butenyl, 2-hexenyl and 3-octenyl groups. One of the double bond carbons can optionally be the point of attachment of the alkenyl substituent. The term “alkynyl” refers to a straight or branched hydrocarbon chain containing 2-20 carbon atoms and having one or more triple bonds. Any atom can be substituted, e.g., by one or more substituents. Alkynyl groups can include, e.g., ethynyl, propargyl, and 3-hexynyl. One of the triple bond carbons can optionally be the point of attachment of the alkynyl substituent.

The term “alkoxy” refers to an —O-alkyl radical. The term “mercapto” refers to an SH radical. The term “thioalkoxy” refers to an —S-alkyl radical. The terms “aryloxy” and “heteroaryloxy” refer to an —O-aryl radical and —O-heteroaryl radical, respectively. The terms “thioaryloxy” and “thioheteroaryloxy” refer to an —S-aryl radical and —S-heteroaryl radical, respectively.

The terms “aralkoxy” and “heteroaralkoxy” refer to an —O-aralkyl radical and —O-heteroaralkyl radical, respectively. The terms “thioaralkoxy” and “thioheteroaralkoxy” refer to an —S-aralkyl radical and —S-heteroaralkyl radical, respectively. The term “cycloalkoxy” refers to an —O-cycloalkyl radical. The terms “cycloalkenyloxy” and “heterocycloalkenyloxy” refer to an —O-cycloalkenyl radical and —O-heterocycloalkenyl radical, respectively. The term “heterocyclyloxy” refers to an —O-heterocyclyl radical. The term “thiocycloalkoxy” refers to an —S-cycloalkyl radical. The terms “thiocycloalkenyloxy” and “thioheterocycloalkenyloxy” refer to an —S-cycloalkenyl radical and —S-heterocycloalkenyl radical, respectively. The term “thioheterocyclyloxy” refers to an —S-heterocyclyl radical.

The term “heterocyclyl” refers to a saturated monocyclic, bicyclic, tricyclic or other polycyclic ring system having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂). For example, carbon atoms and 1-4, 1-8, or 1-10 heteroatoms of N, O, or S if monocyclic, bicyclic, or tricyclic, respectively). The heteroatom or ring carbon is the point of attachment of the heterocyclyl substituent to another moiety. Any atom can be substituted, e.g., by one or more substituents. The heterocyclyl groups can contain fused rings. Fused rings are rings that share a common carbon atom. Heterocyclyl groups can include, e.g., tetrahydrofuryl, tetrahydropyranyl, piperidyl (piperidino), piperazinyl, morpholinyl (morpholino), pyrrolinyl, and pyrrolidinyl.

The term “cycloalkenyl” refers to partially unsaturated monocyclic, bicyclic, tricyclic, or other polycyclic hydrocarbon groups. A ring carbon (e.g., saturated or unsaturated) is the point of attachment of the cycloalkenyl substituent. Any atom can be substituted e.g., by one or more substituents. The cycloalkenyl groups can contain fused rings. Fused rings are rings that share a common carbon atom. Cycloalkenyl moieties can include, e.g., cyclohexenyl, cyclohexadienyl, or norbornenyl.

The term “heterocycloalkenyl” refers to partially unsaturated monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). A ring carbon (e.g., saturated or unsaturated) or heteroatom is the point of attachment of the heterocycloalkenyl substituent. Any atom can be substituted, e.g., by one or more substituents. The heterocycloalkenyl groups can contain fused rings. Fused rings are rings that share a common carbon atom. Heterocycloalkenyl groups can include, e.g., tetrahydropyridyl, dihydropyranyl, 4,5-dihydrooxazolyl, 4,5-dihydro-1H-imidazolyl, 1,2,5,6-tetrahydro-pyrimidinyl, and 5,6-dihydro-2H-[1,3]oxazinyl.

The term “aryl” refers to an aromatic monocyclic, bicyclic, or tricyclic hydrocarbon ring system, wherein any ring atom can be substituted, e.g., by one or more substituents. Aryl groups can contain fused rings. Fused rings are rings that share a common carbon atom. Aryl moieties can include, e.g., phenyl, naphthyl, anthracenyl, and pyrenyl.

The term “heteroaryl” refers to an aromatic monocyclic, bicyclic, tricyclic, or other polycyclic hydrocarbon groups having 1-4 heteroatoms if monocyclic, 1-8 heteroatoms if bicyclic, or 1-10 heteroatoms if tricyclic, said heteroatoms independently selected from O, N, or S (and mono and dioxides thereof, e.g., N→O⁻, S(O), SO₂) (e.g., carbon atoms and 1-4, 1-8, or 1-10 heteroatoms of N, O, or S if monocyclic, bicyclic, or tricyclic, respectively). Any atom can be substituted, e.g., by one or more substituents. Heteroaryl groups can contain fused rings. Fused rings are rings that share a common carbon atom. Heteroaryl groups can include, e.g., pyridyl, thienyl, furyl (furanyl), imidazolyl, indolyl, isoquinolyl, quinolyl and pyrrolyl.

The terms “arylcycloalkyl” and “heteroarylcycloalkyl” refer to bicyclic, tricyclic, or other polycyclic ring systems that include one (or more) aryl or heteroaryl rings, respectively, fused to a cycloalkyl ring. Any atom can be substituted, e.g., by one or more substituents (e.g., R⁸, R¹⁰, or R^{e}). A ring atom on either the aryl portion or the cycloalkyl portion can serve as the point of attachment of the arylcycloalkyl to another moiety. Likewise, a ring atom on either the heteroaryl portion or the cycloalkyl portion can serve as the point of attachment of the heteroarylcycloalkyl to another moiety. Arylcycloalkyl can include, e.g., tetrahydronaphthyl, indanyl (sometimes referred to as dihydroindenyl), and fluorenyl.

The terms “arylcycloalkenyl” and “heteroarylcycloalkenyl” refer to bicyclic, tricyclic, or other polycyclic ring systems that include one (or more) aryl or heteroaryl rings, respectively, fused to a cycloalkenyl ring. Any atom can be substituted, e.g., by one or more substituents (e.g., R⁸, R¹⁰, or R^{e}). A ring atom on either the aryl portion or the cycloalkenyl portion can serve as the point of attachment of the arylcycloalkenyl to another moiety. Likewise, a ring atom on either the heteroaryl portion or the cycloalkenyl portion can serve as the point of attachment of the heteroarylcycloalkenyl to another moiety. Arylcycloalkenyl can include, e.g., dihydronaphthyl,

The terms “arylheterocyclyl” and “heteroarylheterocyclyl” refer to bicyclic, tricyclic, or other polycyclic ring systems that include an aryl or heteroaryl ring, respectively, that is fused to a heterocyclyl ring that includes from 1-3 heteroatoms independently selected from O, N, or S (and mono and dioxides thereof, e.g., N→O⁻, S(O), SO₂). The remaining ring atoms of the heterocyclyl ring are carbon. Any atom can be substituted, e.g., by one or more substituents. A ring atom on either the aryl portion or the heterocyclyl portion can serve as the point of attachment of the arylheterocyclyl to another moiety. Likewise, a ring atom on either the heteroaryl portion or the heterocyclyl portion can serve as the point of attachment of the heteroarylheterocyclyl to another moiety. Arylheterocyclyl can include, e.g., 1,2,3,4-tetrahydroquinolyl, 1,2,3,4-tetrahydroisoquinolyl, isoindolinyl-1,3-dione (phthalimido), dihydrobenzo[b]thienyl, isobenzofuranyl-1-one, and benzo[1,3]dioxolyl.

The terms “arylheterocycloalkenyl” and “heteroarylheterocycloalkenyl” refer to bicyclic, tricyclic, or other polycyclic ring systems that include an aryl or heteroaryl ring, respectively, that is fused to a heterocycloalkenyl ring that includes from 1-3 heteroatoms independently selected from O, N, or S (and mono and dioxides thereof, e.g., N→O⁻, S(O), SO₂). The remaining ring atoms of the heterocycloalkenyl ring are carbon. Any atom can be substituted, e.g., by one or more substituents (e.g., R⁸, R¹⁰, or R^{e}). A ring atom on either the aryl portion or the heterocycloalkenyl portion can serve as the point of attachment of the arylheterocycloalkenyl to another moiety. Likewise, a ring atom on either the heteroaryl portion or the heterocycloalkenyl portion can serve as the point of attachment of the heteroarylheterocycloalkenyl to another moiety. Arylheterocycloalkenyl can include, e.g., isochromenyl-1-one.

The term “oxo” refers to an oxygen atom, which forms a carbonyl (C═O) when attached to carbon, or which forms part of a sulfinyl or sulfonyl group when attached to a sulfur atom, or which forms part of an N-oxide when attached to a nitrogen. The term “thioxo” refers to an oxygen atom, which forms a thiocarbonyl (C═S) when attached to carbon. Descriptors such as C(O), C(S), and C(NRⁱ) refer to carbon atoms that are doubly bonded to an oxygen, sulfur, and nitrogen atom, respectively.

The term “substituent” refers to a group “substituted” on, e.g., an alkyl, cycloalkyl, alkenyl, alkynyl, aralkyl, heteroaralkyl, heterocyclyl, heterocycloalkenyl, cycloalkenyl, aryl, heteroaryl, arylcycloalkyl, heteroarylcycloalkyl, arylcycloalkenyl, heteroarylcycloalkenyl, arylheterocyclyl, heteroarylheterocyclyl, arylheterocycloalkenyl, or heteroarylheterocycloalkenyl group at any atom of that group. In one aspect, the substituent(s) (e.g., R^{a}) on a group are independently any one single, or any combination of two or more of the permissible atoms or groups of atoms delineated for that substituent. In another aspect, a substituent may itself be substituted with any one of the above substituents (e.g., R^{a′}).

In general, when a definition for a particular variable includes both hydrogen and non-hydrogen (halo, alkyl, aryl, etc.) possibilities, the term “substituent(s) other than hydrogen” refers collectively to the non-hydrogen possibilities for that particular variable.

In some embodiments, the compounds have agonist activity for genes involved with HDL production and cholesterol efflux (e.g., ABCA1) and antagonist activity for genes involved with triglyceride synthesis (e.g., SREBP-1c).

The details of one or more embodiments of the invention are set forth in the description below. Other features and advantages of the invention will be apparent from the description and from the claims.

### DETAILED DESCRIPTION

This invention relates generally to quinoline-based modulators of LXRs and related methods and compositions.

The quinoline-based LXR modulators have the general formula (I):

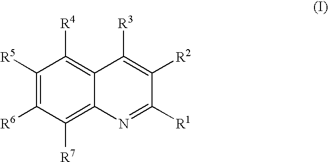

in which R¹, R², R³, R⁴, R⁵, R⁶, R⁷, R⁸, R⁹, R¹⁰, R¹¹, R¹², R¹³, R¹⁴, R¹⁵, R¹⁶, X, W, W¹, W², W³, A, R^{a}, R^{a′, R}^{b}, R^{b′}, R^{c}, R^{d}, R^{d′}, R^{e}, R^{f}, R^{f′}, R^{g}, R^{h}, Rⁱ R^{j}, R^{k}, R^{m}, and n, can be as defined anywhere herein.

For ease of exposition, it is understood that where in this specification (including the claims), a group is defined by “as defined anywhere herein” (or the like), the definitions for that particular group include the first occurring and broadest generic definition as well as any subgeneric and specific definitions delineated anywhere in this specification.

Also, for ease of exposition, it is understood that any recitation of ranges (e.g., C₁-C₁₂, 1-4) or subranges of a particular range (e.g., C₁-C₄, C₂-C₆, 1-2) for any of R¹, R², R³R⁴, R⁵, R⁶, R⁷, R⁸, R⁹, R¹⁰, R¹¹, R¹², R¹³, R¹⁴, R¹⁵, R¹⁶, X, W, W¹, W², W³, A, R^{a}, R^{a′}, R^{b}, R^{b′}, R^{c}, R^{d}, R^{d′}, R^{e}, R^{f}, R^{f′}, R^{g}, R^{h}, Rⁱ R^{j}, R^{k}, R^{m}, and n expressly includes each of the individual values that fall within the recited range, including the upper and lower limits of the recited range. For example, the range C₁-C₄ alkyl is understood to mean C₁, C₂, C₃, or C₄ alkyl or the range 1-3 R^{a} is understood to mean 1, 2, or 3 R^{a}.

Variable R¹

In some embodiments, R¹ can be hydrogen.

Variable R²

In some embodiments, R² can be:

(i) hydrogen; or

(ii) C₁-C₁₂ (e.g., C₁-C₆ or C₁-C₄) alkyl or C₁-C₁₂ (e.g., C₁-C₆ or C₁-C₄) haloalkyl, each of which is optionally substituted with from 1-5 (e.g., 1-4, 1-3, 1-2, 1) R^{a}; or

(iii) C₇-C₂₀ (e.g., C₇-C₁₆, C₇-C₁₂, C₇-C₁₀) aralkyl or heteroaralkyl including 6-20 (e.g., 6-14, 6-12, 6-10) atoms, each of which is optionally substituted with from 1-10 (e.g., 1-5, 1-4, 1-3, 1-2, 1) R^{b}; or

(vi) C₆-C₁₈ (e.g., C₆-C₁₄, C₆-C₁₀, phenyl) aryl or heteroaryl including 5-16 (e.g., 5-14, 5-10, 5-6) atoms, each of which is optionally substituted with from 1-10 (e.g., 1-5, 1-4, 1-3, 1-2, 1) R^{d}.

In some embodiments, R² can be:

(i) hydrogen; or

(ii) C₁-C₁₂ (e.g., C₁-C₆ or C₁-C₄) alkyl or C₁-C₁₂ (e.g., C₁-C₆ or C₁-C₄) haloalkyl, each of which is optionally substituted with from 1-5 (e.g., 1-4, 1-3, 1-2, 1) R^{a}; or

(iii) C₇-C₂₀ (e.g., C₇-C₁₆, C₇-C₁₂, C₇-C₁₀) aralkyl or heteroaralkyl including 6-20

(e.g., 6-14, 6-12, 6-10) atoms, each of which is optionally substituted with from 1-10

(e.g., 1-5, 1-4, 1-3, 1-2, 1) R^{b}.

In certain embodiments, R² can be hydrogen.

In certain embodiments, R² can be C₁-C₆ (e.g., C₁-C₄) alkyl, which is optionally substituted with 1 or 2 R^{a} (e.g., hydroxy). For example, R² can be CH₃ (methyl), ethyl, n-propyl, or iso-propyl. An exemplary R² alkyl substituent is CH₃.

In certain embodiments, R² can be C₇-C₁₀ aralkyl, which is optionally substituted with from 1-3 (e.g., 1-2, 1) R^{b} (e.g., C₁-C₃ alkyl; C₁-C₃ haloalkyl, e.g., C₁-C₃ perfluoroalkyl; C₁-C₃ alkoxy; C₂-C₄ alkenyl; C₂-C₄ alkynyl; hydroxyl; halogen; nitro; or CN). An exemplary R² aralkyl substituent is benzyl.

In some embodiments, when:

(i) R³ is phenyl that is mono-substituted at the meta position with 1 R⁸ (i.e., meta relative to the ring atom that serves as the point of connection of the phenyl ring to the 4-position of the quinoline ring in formula (I)), and each of the remaining ring atoms on the phenyl ring are attached to hydrogen; and

(ii) W is —OCH₂—, —NHCH₂—, —CH₂NH—, or —N(CH₃)CH₂—; and

(iii) A is phenyl that is mono-substituted at the para position with 1 CH₂C(O)OH

(i.e., para relative to the ring atom that serves as the point of connection of the phenyl ring to variable W), and each of the remaining ring atoms on the phenyl ring are attached to hydrogen;

then R² cannot be C₇-C₁₂ (e.g., C₇-C₁₀, C₇) aralkyl (e.g., R² cannot be benzyl under these conditions).

In other embodiments, R² can be C₆-C₁₀ aryl that is optionally substituted with from 1-3 (e.g., 1-2, 1) R^{d} (e.g., C₁-C₃ alkyl; C₁-C₃ haloalkyl, e.g., C₁-C₃ perfluoroalkyl; C₁-C₃ alkoxy; C₂-C₄ alkenyl; C₂-C₄ alkynyl; hydroxyl; halogen; nitro; or CN). For example, R² can be phenyl optionally substituted with from 1-3 R^{d}.

In still other embodiments, R² can be C₂-C₁₂ (e.g., C₂-C₆, C₂-C₄) alkenyl; C₂-C₁₂ (e.g., C₂-C₆, C₂-C₄) alkynyl; or C₃-C₁₀ (e.g., C₃-C₆) cycloalkyl.

Variable R³

In some embodiments, R³ can be C₆-C₁₈ (e.g., C₆-C₁₄, C₆-C₁₀, C₆) aryl, which is (i) substituted with from 1-5 (e.g., 1-4, 1-3, 1-2, 1) R⁸ and (ii) optionally substituted with from 1-4 (e.g., 1-3, 1-2, 1) R^{e}.

By way of example, when R³ is aryl and substituted with R^{e}, each R^{e} can be independently of one another: C₁-C₃ alkyl, optionally substituted with from 1-3 R^{a} (e.g., hydroxyl or —C(O)OR^{j}, e.g., the alkyl group can be CH₂C(O)OR^{j}); C₁-C₃ haloalkyl (e.g., C₁-C₃ fluoroalkyl, e.g., 1-5 fluorines can be present; or C₁-C₃ perfluoroalkyl); halo; CN; mercapto; C₁-C₆ thioalkoxy optionally substituted with from 1-3 R^{a}; C₆-C₁₀ aryl (e.g., phenyl) or C₆-C₁₀ aryloxy (e.g., phenoxy), each of which is optionally substituted with from 1-10 R^{d}; hydroxyl; NR^{g}R^{h} (e.g., NH₂, monoalkylamino, or dialkylamino); nitro; C₂-C₄ alkenyl; C₂-C₄ alkynyl; C₁-C₃ alkoxy; C₁-C₃ haloalkoxy; —C(O)OR^{j} (e.g., R^{j} can be hydrogen or C₁-C₃ alkyl); or —C(O)R^{j} (e.g., R^{j} can be C₁-C₃ alkyl).

In some embodiments, R³ can be C₆-C₁₀ aryl, which is (i) substituted with from 1-5 (e.g., 1-4, 1-3, 1-2, 1) R⁸ and (ii) optionally substituted with from 1-4 (e.g., 1-3, 1-2, 1) R^{e}.

In some embodiments, R³ can be C₆-C₁₀ aryl, which is (i) substituted with 1 or 2 R⁸ and (ii) optionally substituted with 1 or 2 R^{e}.

In some embodiments, R³ can be naphthyl, which is (i) substituted with 1 or 2 R⁸ and (ii) optionally substituted with 1 or 2 R^{e}.

In some embodiments, R³ can be phenyl, which is (i) substituted with 1 or 2 R⁸ and (ii) optionally substituted with 1 or 2 R^{e}.

In certain embodiments, R³ can be phenyl, which is (i) substituted with 1 R⁸ and (ii) optionally substituted with 1 or 2 R^{e}. For example, R³ can be phenyl, which is substituted with 1 R⁸. In these embodiments, R⁸ (i.e., the moiety —WA) can be attached to a ring carbon that is ortho, meta, or para (preferably meta) with respect to the ring carbon that connects the phenyl ring to the 4-position of the quinoline ring, and R^{e}, when present can be connected to ring carbons that are not occupied by WA.

In some embodiments, R³ can be heteroaryl including 5-16 (e.g., 5-14, 5-10, 5-6) atoms, which is (i) substituted with from 1-5 (e.g., 1-4, 1-3, 1-2, 1) R⁸ and (ii) optionally substituted with from 1-4 (e.g., 1-3, 1-2, 1) R^{e}.

By way of example, when R³ is heteroaryl and substituted with R^{e}, each R^{e} can be independently of one another: C₁-C₃ alkyl, optionally substituted with from 1-3 R^{a}; C₁-C₃ haloalkyl (e.g., C₁-C₃ fluoroalkyl, e.g., 1-5 fluorines can be present; or C₁-C₃ perfluoroalkyl); halo; CN; mercapto; C₁-C₆ thioalkoxy optionally substituted with from 1-3 R^{a}; hydroxyl; NR^{g}R^{h} (e.g., NH₂, monoalkylamino, or dialkylamino); nitro; C₁-C₃ alkoxy; C₁-C₃ haloalkoxy; or —C(O)R^{j} (e.g., R^{j} can be C₁-C₃ alkyl).

In some embodiments, R³ can be heteroaryl including 5-12 (e.g., 5-10) atoms, which is (i) substituted with from 1-4 (e.g., 1-3, 1-2, 1) R⁸ and (ii) optionally substituted with from 1-4 (e.g., 1-3, 1-2, 1) R^{e}.

In some embodiments, R³ can be heteroaryl including 5-12 (e.g., 5-10) atoms, which is (i) substituted 1 or 2 R⁸ and (ii) optionally substituted with 1 or 2 R^{e}.

In some embodiments, R³ can be heteroaryl including 5-6 atoms, which is (i) substituted 1 or 2 R⁸ and (ii) optionally substituted with 1 or 2 R^{e}.

In some embodiments, R³ can be heteroaryl including 8-10 atoms, which is (i) substituted 1 or 2 R⁸ and (ii) optionally substituted with 1 or 2 R^{e}.

In certain embodiments, R³ can be pyridyl, pyrimidinyl, thienyl, furyl, quinolinyl, oxazolyl, thiazolyl, imidazolyl, isoxazolyl, indolyl, benzo[1,3]-dioxolyl, benzo[1,2,5]-oxadiazolyl, isochromenyl-1-one, 3-H-isobenzofuranyl-1-one (e.g., pyridyl, thienyl, or indolyl, e.g., pyridyl), each of which is (i) substituted with 1 R⁸ and (ii) optionally substituted with 1 or 2 R^{e}. For example, R³ can be pyridyl substituted with 1 R⁸.

In some embodiments, R³ can have formula (A):

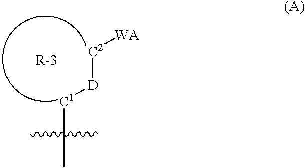

in which:

(i) ring (R-3) is C₆-C₁₀ aryl or heteroaryl including 5-10 atoms, each of which can be optionally further substituted with 1 or 2 R^{e}; and

(ii) D is N, NR^{g} (e.g., NH), O, S, CH, or CR^{e}.

Thus, in these embodiments, there is an intervening ring atom (i.e., D) between the ring carbon that is attached to WA (i.e., C²) and the ring carbon that connects the ring R-3 to the 4-position of the quinoline ring (i.e., C¹). For purposes of clarification, atoms C¹, D, and C² form part of the aryl or heteroaryl ring system. While not shown expressly in formula (A), it is understood that these three atoms can be linked together either by two single bonds or a double bond and a single bond. Each of W and A can be as defined anywhere herein.

In certain embodiments, R³ can have formula (A-1):

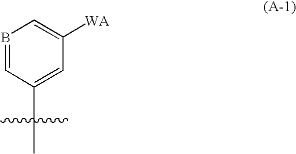

in which B is N, CH, or CR^{e}, and each of W and A can be as defined anywhere herein.

For example, R³ can have formula (A-2) or (A-3):

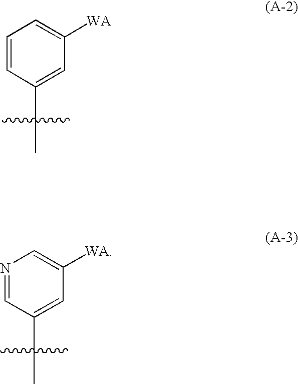

Variable W

In some embodiments, W can be —(C₁₋₆ alkylene)W¹—. In certain embodiments, W¹ is —NR⁹—, in which R⁹ can be hydrogen; or W¹ can be —O—. In certain embodiments, W is —(C₁₋₃ alkylene)NH— (e.g., —CH₂NH—). In certain embodiments, W is —(C₁₋₃ alkylene)O— (e.g., —CH₂O—).

In other embodiments, W can be —W¹(C₁₋₆ alkylene)-. In certain embodiments, W¹ can be —O—. For example, W can be —O(C₁₋₃ alkylene)- (e.g., —OCH₂—).

In still other embodiments, W can be a bond; —O—; C₂-C₄ alkynylene (e.g., —C≡C—); or C₁₋₃ alkylene (e.g., CH₂).

Variable A

In general, A is a cyclic group that is (a) substituted with one or more R¹⁰; and (b) optionally substituted with one or more R^{e}.

In some embodiments, A can be:

(i-A) C₆-C₁₀ (e.g., phenyl) aryl, which is (a) substituted with from 1-5 (e.g., 1-4, 1-3, 1-2, 1, e.g., 1) R¹⁰; and (b) optionally substituted with from 1-6 (e.g., 1-5, 1-4, 1-3, 1-2, 1, e.g., 1-2) R^{e};

and/or

(ii-A) C₈-C₂₀ (e.g., C₈-C₁₂, C₁₀) arylcycloalkenyl, which is (a) substituted with from 1-5 (e.g., 1-4, 1-3, 1-2, 1, e.g., 1) R¹⁰; and (b) optionally substituted with from 1-6 (e.g., 1-5, 1-4, 1-3, 1-2, 1, e.g., 1-2) R^{e};

and/or

(iii-A) C₈-C₂₀ (e.g., C₈-C₁₂, C₁₀) arylcycloalkyl, which is (a) substituted with from 1-5 (e.g., 1-4, 1-3, 1-2, 1, e.g., 1) R¹⁰; and (b) optionally substituted with from 1-6 (e.g., 1-5, 1-4, 1-3, 1-2, 1, e.g., 1-2) R^{e};

and/or

(iv-A) heteroaryl including 5-12 (e.g., 5-10, 5-8) atoms, which is (a) substituted with from 1-5 (e.g., 1-4, 1-3, 1-2, 1, e.g., 1) R¹⁰; and (b) is optionally substituted with from 1-4 (e.g., 1-3, 1-2, 1, e.g., 1-3) R^{e}.

In embodiments, any one of the following combinations can apply for defining A:
(i-A), (ii-A), or (iii-A); or
(i-A) and (ii-A), or (i-A) and (iii-A), or (iii-A) and (iv-A); or
(i-A), (ii-A), and (iii-A).

In some embodiments, A can be C₆-C₁₀ aryl, which is (i) substituted with 1 or 2 R¹⁰ and (ii) optionally substituted with from 1-6 (e.g., 1-5, 1-4, 1-3, 1-2, 1, e.g., 1-2) R^{e}.

By way of example, when A is aryl (or arylcycloalkenyl or arylcycloalkyl) and substituted with R^{e}, each R^{e} can be independently of one another: C₁-C₃ alkyl, optionally substituted with from 1-3 R^{a} (e.g., hydroxyl or —C(O)OR^{j}, e.g., the alkyl group can be CH₂C(O)OR^{j}); C₁-C₃ haloalkyl (e.g., C₁-C₃ fluoroalkyl, e.g., 1-5 fluorines can be present; or C₁-C₃ perfluoroalkyl); halo; CN; mercapto; C₁-C₆ thioalkoxy optionally substituted with from 1-3 R^{a}; C₆-C₁₀ aryl (e.g., phenyl) or C₆-C₁₀ aryloxy (e.g., phenoxy), each of which is optionally substituted with from 1-10 R^{d}; hydroxyl; NR^{g}R^{h} (e.g., NH₂, monoalkylamino, or dialkylamino); nitro; C₂-C₄ alkenyl; C₂-C₄ alkynyl; C₁-C₃ alkoxy; C₁-C₃ haloalkoxy; —C(O)OR^{j} (e.g., R^{j} can be hydrogen or C₁-C₃ alkyl); or —C(O)R^{j} (e.g., R^{j} can be C₁-C₃ alkyl).

In some embodiments, A can be C₆-C₁₀ aryl, which is (i) substituted with 1 R¹⁰ and (ii) optionally substituted with from 1-6 (e.g., 1-5, 1-4, 1-3, 1-2, 1, e.g., 1-2) R^{e}.

In some embodiments, A can be phenyl, which is (i) substituted with 1 R¹⁰ and (ii) optionally substituted with from 1-4 (e.g., 1-3, 1-2, 1) R^{e}.

In some embodiments, A can be phenyl, which is (i) substituted with 1 R¹⁰ and (ii) substituted with from 1-4 (e.g., 1-3, 1-2, 1) R^{e}.

In these embodiments, R¹⁰ can be attached to a ring carbon that is ortho, meta, or para (preferably para) with respect to the ring carbon that connects the phenyl ring to W.

In certain embodiments, A can have formula (B-1):

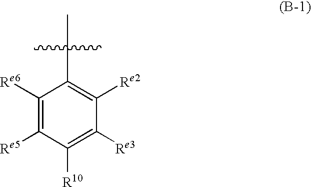

in which each of R^{e2}, R^{e3}, R^{e5}, and R^{e6} is, independently, hydrogen or R^{e}. Each of R^{e} and R¹⁰ can be as defined anywhere herein. In certain embodiments, each of R^{e2}, R^{e3}, R^{e5}, and R^{e6} can be hydrogen. In certain embodiments, one or two (e.g., two) of R^{e2}, R^{e3}, R^{e5}, and R^{e6} is (or are each independently) R^{e}, and the others are hydrogen. In certain embodiments, each of R^{e2}, R^{e3}, R^{e5} and R^{e6} is, independently, R^{e} (i.e., a substituent other than hydrogen).

In some embodiments, A can be naphthyl, which is (i) substituted with 1 R¹⁰ and (ii) substituted with from 1-6 (e.g., 1-5, 1-4, 1-3, 1-2, 1, e.g., 1-2) R^{e}.

In certain embodiments, A can have formula (B-2):

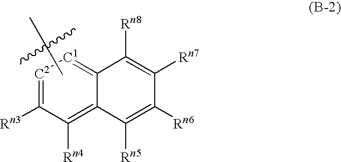

in which each of Rⁿ³ and Rⁿ⁴ is, independently, hydrogen or R^{e} (e.g., each can be hydrogen); and one of Rⁿ⁵, Rⁿ⁶, Rⁿ⁷, and Rⁿ⁸ is R¹⁰, and the others are each, independently, hydrogen or R^{e} (e.g., each can be hydrogen). Each of R^{e} and R¹⁰ can be as defined anywhere herein. In these embodiments, the ring carbon that is not the point of attachment of the naphthyl ring to W (i.e., C¹ or C² in formula B-2) can be attached to a hydrogen atom or to R^{e} (e.g., hydrogen). In certain embodiments, C¹ can be the point of attachment of the naphthyl ring to W, and Rⁿ⁵ can be R¹⁰. In certain embodiments, C² can be the point of attachment of the naphthyl ring to W, and Rⁿ⁶ can be R¹⁰.

In some embodiments, A can be C₈-C₁₂ arylcycloalkenyl, which is (i) substituted with 1 R¹⁰ and (ii) optionally substituted with from 1-6 (e.g., 1-5, 1-4, 1-3, 1-2, 1, e.g., 1-2) R^{e}. For example, A can be dihydronaphthyl.

In other embodiments, A can be C₈-C₁₂ arylcycloalkyl, which is (i) substituted with 1 R¹⁰ and (ii) optionally substituted with from 1-6 (e.g., 1-5, 1-4, 1-3, 1-2, 1, e.g., 1-2) R^{e}. For example, A can be tetrahydronaphthyl or indanyl.

In still other embodiments, A can be pyridyl, pyrimidinyl, thienyl, furyl, quinolinyl, oxazolyl, thiazolyl, imidazolyl, isoxazolyl, indolyl, benzo[1,3]-dioxolyl, benzo[1,2,5]-oxadiazolyl, isochromenyl-1-one, 3-H-isobenzofuranyl-1-one (e.g., pyridyl, thienyl, or indolyl, e.g., pyridyl), which is (i) substituted with 1 R¹⁰ and (ii) optionally substituted with 1 or 2 R^{e}. By way of example, when R^{e} is present, each R^{e} can be independently of one another: C₁-C₃ alkyl, optionally substituted with from 1-3 R^{a}; C₁-C₃ haloalkyl (e.g., C₁-C₃ fluoroalkyl, e.g., 1-5 fluorines can be present; or C₁-C₃ perfluoroalkyl); halo; CN; mercapto; C₁-C₆ thioalkoxy optionally substituted with from 1-3 R^{a}; hydroxyl; NR^{g}R^{h} (e.g., NH₂, monoalkylamino, or dialkylamino); nitro; C₁-C₃ alkoxy; C₁-C₃ haloalkoxy; or —C(O)R^{j} (e.g., R^{j} can be C₁-C₃ alkyl).

Variable R¹⁰

R¹⁰ can be:

(i) —W²—C(O)OR¹¹; and/or

(ii)

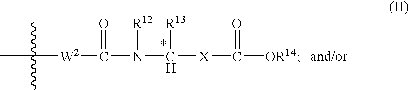

(iii) —W²—C(O)NR¹⁵R¹⁶.

In embodiments, any one of the following combinations can apply for defining R¹⁰:
(i), (ii), or (iii); or
(i) and (ii), or (i) and (iii), or (ii) and (iii); or
(i), (ii), and (iii).

In some embodiments, R¹⁰ can be —W²—C(O)OR¹¹.

In some embodiments, R¹¹ can be:

(i) hydrogen; or

(ii) C₁-C₁₀ (e.g., C₁-C₇) alkyl, which is optionally substituted with from 1-3 (e.g., 1-2, 1) R^{a}; or

(iii) C₂-C₁₂ (e.g., C₃-C₁₂, C₃-C₇) alkenyl or C₂-C₁₂ (e.g., C₃-C₁₂, C₃-C₇) alkynyl, each of which is optionally substituted with from 1-3 (e.g., 1-2, 1) R^{c}; or

(iv) C₃-C₇ cycloalkyl or C₇-C₁₂ aralkyl, each of which is optionally substituted with from 1-10 R^{b}.

In certain embodiments, R¹¹ can be hydrogen.

In some embodiments, W² can be C₁-C₆ alkylene, optionally substituted with from 1-3 R^{b}; or a bond.

In certain embodiments, W² can be C₁-C₆ alkylene. For example, W² can be C₁-C₃ alkylene, such as CH₂ or CH₂CH₂.

In certain embodiments, W² can be a bond.

In some embodiments, R¹⁰ can have formula (C):

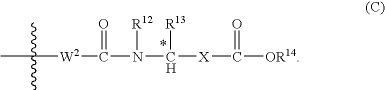

In some embodiments, W² can be a bond.

In some embodiments, X can be a bond.

In some embodiments, R¹² can be hydrogen.

In some embodiments, R¹⁴ can be hydrogen. In these embodiments, R¹⁰ can be a zwitterion.

In some embodiments, each of R¹² and R¹⁴ can be hydrogen.

In some embodiments, X can be a bond, and each of R¹² and R¹⁴ can be hydrogen.

In some embodiments, W² can be a bond, X can be a bond, and each of R¹² and R¹⁴ can be hydrogen.

In some embodiments, when R¹³ is a substituent other than hydrogen, *C can have the R or the S configuration. In some embodiments, when R¹³ is a substituent other than hydrogen, some of the population of *C can have the R configuration, and some of the population of *C can have the S configuration (e.g., about 50% of the population of *C can have the R configuration, and about 50% of the population of *C can have the S configuration).

In some embodiments, R¹³ can be a substituent other than hydrogen.

In certain embodiments, R¹³ can be:

(ii) C₁-C₂₀ alkyl or C₁-C₂₀ haloalkyl, each of which is optionally substituted with from 1-10 R^{a}; or

(iv) C₇-C₂₀ aralkyl, or heteroaralkyl including 6-20 atoms, each of which is optionally substituted with from 1-10 R^{b}; or

(v) C₆-C₁₈ aryl or heteroaryl including 5-16 atoms, each of which is optionally substituted with from 1-10 R^{d}.

In certain embodiments, R¹³ can be C₁-C₆ alkyl optionally substituted with from 1-2 R^{a}; or heteroaralkyl including 6-20 atoms optionally substituted with from 1-2 R^{b}.

In embodiments, when R¹³ includes ionizable substituents, these substituents can be in charged or uncharged form.

By way of example, R¹³ can be the sidechain that is present in one of the following amino acids: alanine, valine, leucine, isoleucine, phenylalanine, tryptophan, tyrosine, histidine, serine, threonine, methionine, cysteine, aspartic acid, glutamic acid, asparagines, glutamine, lysine, or arginine.

In some embodiments, R¹⁰ can be —W²—C(O)NR¹⁵R¹⁶.

In some embodiments, W² can be a bond.

In some embodiments, R¹⁵ and R¹⁶ together with the nitrogen atom to which each is attached is heterocyclyl including 5-6 atoms, which is: (i) substituted with from 1 R^{f}; and (ii) optionally substituted with from 1-2 R^{e}.

In certain embodiments, R¹⁵ and R¹⁶ together with the nitrogen atom to which each is attached is piperidin-1-yl or pyrrolidin-1-yl, which is: (i) substituted with from 1 R^{f}; and (ii) optionally substituted with from 1-2 R^{e}.

In some embodiments, R^{f} can be —X—C(O)OH. In these embodiments, R¹⁰ can be a zwitterion. In some embodiments, X can be a bond (i.e., R^{f} is —C(O)OH).

In some embodiments, when the carbon attached to R^{f} is a stereogenic carbon, the stereogenic carbon can have the R or the S configuration. In some embodiments, some of the stereogenic carbon population can have the R configuration, and some can have the S configuration (e.g., about 50% of the stereogenic carbon population can have the R configuration, and about 50% of the stereogenic carbon population can have the S configuration).

Variables R⁴, R⁵, R⁶, and R⁷

In some embodiments, each of R⁴, R⁵ and R⁶ can be, independently of one another, hydrogen, halo (e.g., fluoro), or C₁-C₃ alkyl (e.g., CH₃). In certain embodiments, each of R⁴, R⁵ and R⁶ can be hydrogen.

In some embodiments, R⁷ can be hydrogen, halo, cyano, nitro, C₁-C₁₀ (e.g., C₁-C₆, C₁-C₃) alkyl, or C₁-C₁₀ (e.g., C₁-C₆, C₁-C₃) haloalkyl, or SO₂R^{m}.

In some embodiments, R⁷ can be hydrogen; chloro or bromo (e.g., chloro); cyano, nitro, C₁-C₁₀ (e.g., C₁-C₆, C₁-C₃) alkyl, or C₁-C₁₀ (e.g., C₁-C₆, C₁-C₃) haloalkyl, or SO₂R^{m}.

In some embodiments, R⁷ can be halo, cyano, nitro, C₁-C₁₀ (e.g., C₁-C₆, C₁-C₃) alkyl, or C₁-C₁₀ (e.g., C₁-C₆, C₁-C₃) haloalkyl, or SO₂R^{m}.

In some embodiments, R⁷ can be chloro or bromo (e.g., chloro); cyano, nitro, C₁-C₁₀ (e.g., C₁-C₆, C₁-C₃) alkyl, or C₁-C₁₀ (e.g., C₁-C₆, C₁-C₃) haloalkyl, or SO₂R^{m}.

In some embodiments, R⁷ can be halo, C₁-C₁₀ (e.g., C₁-C₆, C₁-C₃) alkyl, or C₁-C₁₀ (e.g., C₁-C₆, C₁-C₃) haloalkyl, or SO₂R^{m}.

In some embodiments, R⁷ can be chloro or bromo (e.g., chloro); C₁-C₁₀ (e.g., C₁-C₆, C₁-C₃) alkyl, or C₁-C₁₀ (e.g., C₁-C₆, C₁-C₃) haloalkyl, or SO₂R^{m}.

In certain embodiments, R⁷ can be hydrogen; chloro; cyano; CH₃; CF₃; or SO₂CH₃. In certain embodiments, R⁷ can be chloro; cyano; CH₃; CF₃; or SO₂CH₃. In certain embodiments, R⁷ can be chloro; CH₃; CF₃; or SO₂CH₃.

In certain embodiments, R⁷ can be C₁-C₆ (e.g., C₁-C₃) haloalkyl (e.g., CF₃).

In certain embodiments, R⁷ can be halo (e.g., chloro).

In certain embodiments, R⁷ can be SO₂R^{m} (e.g., R^{m} can be CH₃).

In certain embodiments, R⁷ can be hydrogen or cyano.

In some embodiments, R⁷ is other than fluoro.

In some embodiments, the compounds can have formula (II):

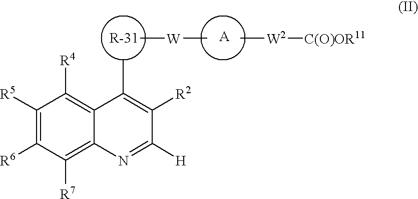

in which:

(i) ring (R-31) can be C₆-C₁₈ aryl, heteroaryl including 5-16 atoms, C₈-C₂₀ arylcycloalkyl, heteroarylcycloalkyl including 8-20 atoms, C₈-C₂₀ arylcycloalkenyl, heteroarylcycloalkenyl including 8-20 atoms, arylheterocyclyl including 8-20 atoms, heteroarylheterocyclyl including 8-20 atoms, arylheterocycloalkenyl including 8-20 atoms, heteroarylheterocycloalkenyl including 8-20 atoms, each of which can be optionally substituted with from 1-4 R^{e}; and

(ii) each of R², R⁴, R⁵, R⁶, R⁷, W, A, W², and R¹¹ can be as defined anywhere herein.

Thus, in these embodiments and in the following subgenera, A is substituted with 1 R¹⁰, which is W²—C(O)OR¹¹.

In certain embodiments, the compounds can have formula (III):

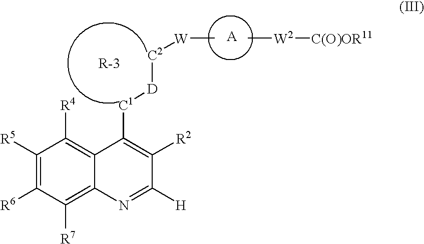

in which R², R⁴, R⁵, R⁶, R⁷, ring (R-3), D, W, A, W², and R¹¹ can be as defined anywhere herein.

In certain embodiments, the compounds can have formula (IV):

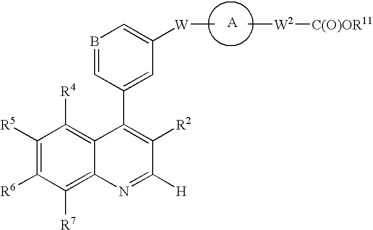

in which R², R⁴, R⁵, R⁶, R⁷, B, W, A, W², and R¹¹ can be as defined anywhere herein.

In some embodiments, compounds having formula (I), (II), (III), or (IV) can include one or more of the following features:

R² can be (i) hydrogen; or (ii) C₁-C₁₂ alkyl which is optionally substituted with from 1-5 R^{a}; or (iii) C₇-C₂₀ aralkyl, which is optionally substituted with from 1-10 R^{b}.

R² is hydrogen.

R² can be C₁-C₆ alkyl, optionally substituted with from 1-2 R^{a}. For example, R² can be CH₃.

R² can be C₇-C₁₀ aralkyl, optionally substituted with from 1-3 R^{b}. For example, R² can be benzyl.

W can be —(C₁₋₆ alkylene)W¹—, and W¹ can be —O— or —NR⁹—, wherein R⁹ is hydrogen or C₁-C₃ alkyl. In certain embodiments, R⁹ can be hydrogen. For example, W can be —(C₁₋₃ alkylene)NR⁹—, in which R⁹ can be hydrogen (e.g., W is —CH₂NH—). As another example, W can be —(C₁₋₃ alkylene)O— (e.g., W can be —CH₂O—).

A can be C₆-C₁₀ aryl, which can optionally further substituted with from 1-4 (e.g., 1-3, 1-2, 1, e.g., 1-2) R^{e}. A can be phenyl that is substituted with from 1-4 (e.g., 1-3, 1-2, 1, e.g., 1-2) R^{e}. For example, A can have the substitution pattern delineated in formula (B-1) or (B-2) described herein and their accompanying definitions.

A can be can be C₈-C₁₂ arylcycloalkenyl, which can be further optionally substituted with from 1-6 (e.g., 1-5, 1-4, 1-3, 1-2, 1, e.g., 1-2) R^{e}. For example, A can be dihydronaphthyl.

W² can be a bond or C₁₋₆ alkylene. W² can be C₁-C₃ alkylene (e.g., CH₂). W² can be a bond.

R¹¹ can be hydrogen.

Each of R⁴, R⁵, and R⁶ can be hydrogen.

R⁷ can be C₁-C₄ haloalkyl (e.g., CF₃).

The compound can have an LXRα/LXRβ binding ratio of from about 9 to about 19; from about 20 to about 27; from about 28 to about 44 (e.g., from about 28 to about 38); or from about 45 to about 50.

In certain embodiments, the compounds can have formula (V):

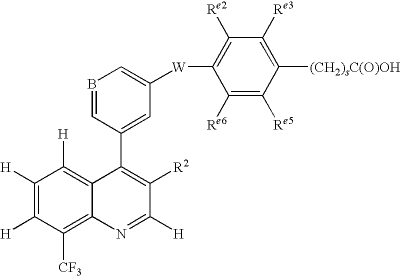

in which:

each of R^{e2}, R^{e3}, R^{e5}, and R^{e6} is, independently, hydrogen, or R^{e};

B can be CH or N;

s can be 0 or 1;

R² can be (i) hydrogen; or (ii) C₁-C₆ alkyl (e.g., CH₃), which is optionally substituted with from 1-5 R^{a}; or (iii) C₇-C₁₀ aralkyl (e.g., benzyl), which is optionally substituted with from 1-10 R^{b}; and

W can be —(C₁₋₃ alkylene)NR⁹—, in which R⁹ can be hydrogen (e.g., W can be —CH₂NH—); or W can be —(C₁₋₃ alkylene)O— (e.g., W can be —CH₂O—).

Embodiments can include one or more of the following features.

R^{e} at each occurrence can be, independently, C₁-C₃ alkyl, optionally substituted with from 1-3 R^{a} (e.g., hydroxyl or —C(O)OR^{j}, e.g., the alkyl group can be CH₂C(O)OR^{j}); C₁-C₃ haloalkyl (e.g., C₁-C₃ fluoroalkyl, e.g., 1-5 fluorines can be present; or C₁-C₃ perfluoroalkyl); halo; CN; mercapto; C₁-C₆ thioalkoxy optionally substituted with from 1-3 R^{a}; C₆-C₁₀ aryl (e.g., phenyl) or C₆-C₁₀ aryloxy (e.g., phenoxy), each of which is optionally substituted with from 1-10 R^{d}; hydroxyl; NR^{g}R^{h} (e.g., NH₂, monoalkylamino, or dialkylamino); nitro; C₂-C₄ alkenyl; C₂-C₄ alkynyl; C₁-C₃ alkoxy; C₁-C₃ haloalkoxy; —C(O)OR^{j} (e.g., R^{j} can be hydrogen or C₁-C₃ alkyl); or —C(O)R^{j} (e.g., R^{j} can be C₁-C₃ alkyl).

One or two of R^{e2}, R^{e3}, R^{e5}, and R^{e6} are each, independently, R^{e}, and the others are hydrogen.

Two of R^{e2}, R^{e3}, R^{e5}, and R^{e6} (e.g., R^{e2} and R^{e3} or R^{e2} and R^{e5}) are each, independently, R^{e}, and the others are hydrogen. In certain embodiments, two of R^{e2}, R^{e3}, R^{e5}, and R^{e6} (e.g., R^{e2} and R^{e3} or R^{e2} and R^{e5}) can each be, independently of one another, C₁-C₃ alkyl, C₁-C₃ alkoxy, or halo. In certain embodiments, two of R^{e2}, R^{e3}, R^{e5}, and R^{e6} (e.g., R^{e2} and R^{e3} or R^{e2} and R^{e5}) can each be, independently of one another, C₁-C₄ alkyl. As an example, two of R^{e2}, R^{e3}, R^{e5}, and R^{e6} (e.g., R^{e2} and R^{e3} or R^{e2} and R^{e5}) can each be CH₃. As another example, two of R^{e2}, R^{e3}, R^{e5} and R^{e6} (e.g., R^{e2} and R^{e3} or R^{e2} and R^{e5}) can each be a different C₁-C₄ alkyl group.

In certain embodiments, the compounds can have formula (VI):

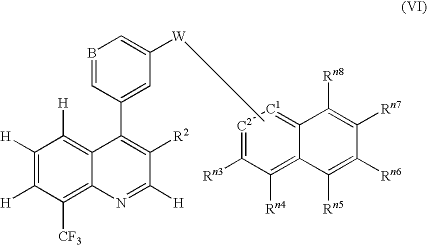

in which each of Rⁿ³, Rⁿ⁴, Rⁿ⁷, and Rⁿ⁸ is, independently, hydrogen or R^{e}; and one of Rⁿ⁵ and Rⁿ⁶ is —(CH₂)ₛC(O)OH, wherein s is 0 or 1, and the other is hydrogen;

B can be CH or N;

R² can be (i) hydrogen; or (ii) C₁-C₆ alkyl (e.g., CH₃), which is optionally substituted with from 1-5 R^{a}; or (iii) C₇-C₁₀ aralkyl (e.g., benzyl), which is optionally substituted with from 1-10 R^{b}; and

W can be —(C₁₋₃ alkylene)NR⁹—, in which R⁹ can be hydrogen (e.g., W can be —CH₂NH—); or W can be —(C₁₋₃ alkylene)O— (e.g., W can be —CH₂O—).

In these embodiments, the ring carbon that is not the point of attachment of the naphthyl ring to W (i.e., C¹ or C² in formula VI) can be attached to a hydrogen atom or to R^{e}.

In certain embodiments, C¹ can be the point of attachment of the naphthyl ring to W, and Rⁿ⁵ can be R¹⁰. In embodiments, C² can be attached to hydrogen, and each of Rⁿ³, Rⁿ⁴, Rⁿ⁷, and Rⁿ⁸ can be hydrogen.

In certain embodiments, C² can be the point of attachment of the naphthyl ring to W, and Rⁿ⁶ can be R¹⁰. In embodiments, C¹ can be attached to hydrogen, and each of Rⁿ³, Rⁿ⁴, Rⁿ⁷, and Rⁿ⁸ can be hydrogen.

R^{e} can be as defined anywhere herein.

In embodiments, the compounds of formula (V) and (VI) can have an LXRα/LXRβ binding ratio of from about 9 to about 19; from about 20 to about 27; from about 28 to about 44 (e.g., from about 28 to about 38); or from about 45 to about 50.

It is understood that the actual electronic structure of some chemical entities cannot be adequately represented by only one canonical form (i.e. Lewis structure). While not wishing to be bound by theory, the actual structure can instead be some hybrid or weighted average of two or more canonical forms, known collectively as resonance forms or structures. Resonance structures are not discrete chemical entities and exist only on paper. They differ from one another only in the placement or “localization” of the bonding and nonbonding electrons for a particular chemical entity. It can be possible for one resonance structure to contribute to a greater extent to the hybrid than the others. Thus, the written and graphical descriptions of the embodiments of the present invention are made in terms of what the art recognizes as the predominant resonance form for a particular species.

The compounds described herein can be synthesized according to methods described herein (or variations thereof) and/or conventional, organic chemical synthesis methods from commercially available starting materials and reagents or from starting materials and reagents that can be prepared according to conventional organic chemical synthesis methods. The compounds described herein can be separated from a reaction mixture and further purified by a method such as column chromatography, high-pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In some embodiments, the compounds described herein can be prepared according to Scheme 1. Condensation of acrylates 2 with various anilines 1 followed by thermal cyclization can provide 4-hydroxyquinoline 3. Conversion of 3 to bromide or chloride 4 can be accomplished using, e.g., phosphorus oxybromide or phosphorus oxychloride, respectively. Reaction of 4 with phenylboronic acids under Suzuki conditions can provide 5. Alkylation of 5 (if X═OH, CH₂OH, NH₂) or reductive amination with 5 (if X═CHO or NH₂) can afford 6. If 6 is a carboxylic ester the corresponding acid is obtained by hydrolysis of the ester. Conversion of acid 6 (R¹¹═H) into amino acid amide 7 can be achieved by conventional amide coupling conditions.

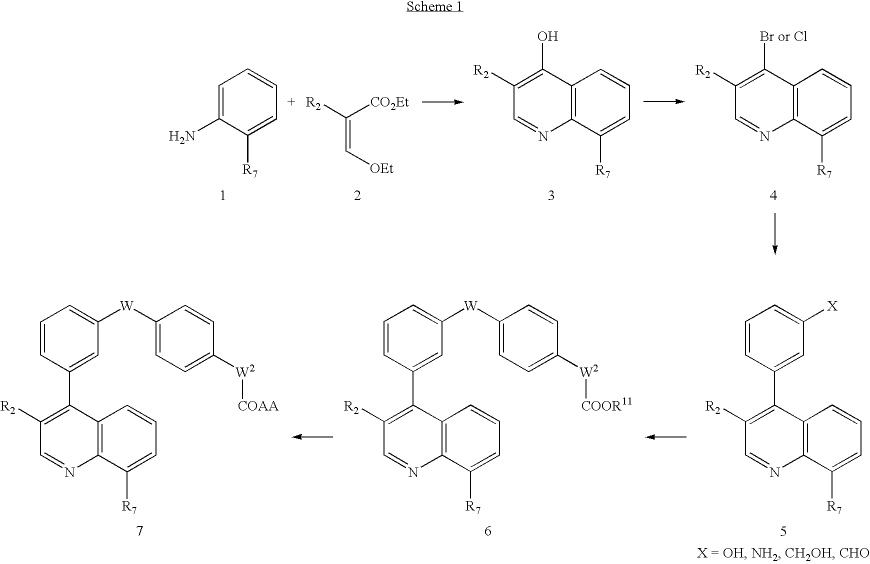

In some embodiments, the alkyl substituted phenyl acetic acids 12 employed as starting materials in the preparation of compounds of formula 14 can be prepared according to Scheme 2. Aniline 8 can be acylated with acetyl chloride. Friedel-Craft acylation of 9 can provide acetamidoacetophone 10. Mono or dialkyl substituted thioacetomorpholide 11 can be prepared by reacting 10 with sulfur and morpholine at temperatures of 100 to 130° C. for a period of 1 to 10 hours. Hydrolysis of 11 with an aqueous solution of an inorganic acid such as hydrochloric acid can give amino phenyl acetic acid 12. Treatment of 12 with aldehyde 13 (Y═CHO) and a reducing agent such as NaBH(OAc)₃, can result in the secondary amine product of formula 14. The same product of formula 14 could also be obtained upon treating the starting primary amine with an alkylating agent 13 (Y═CH₂Br) in the presence of a base.

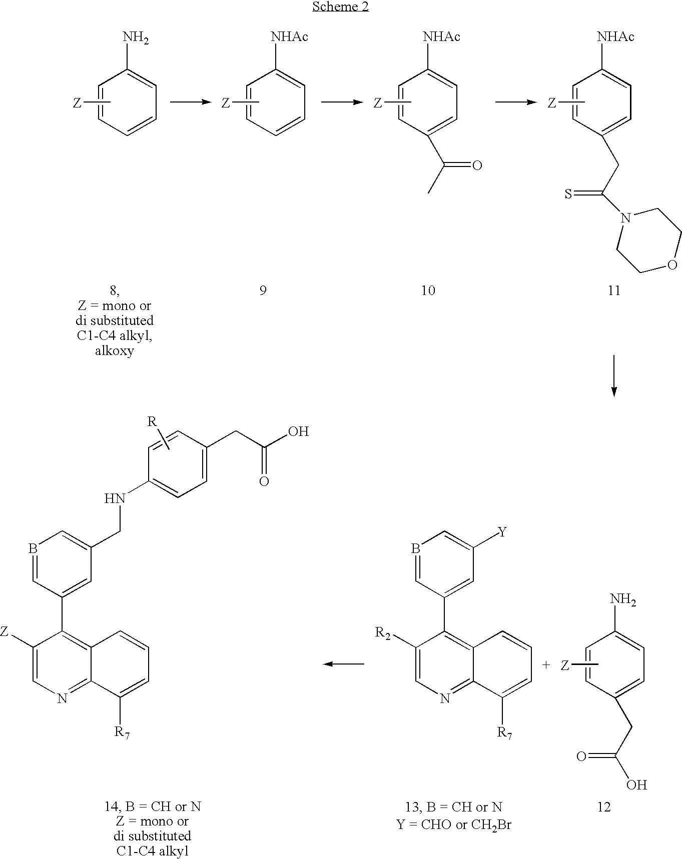

In other embodiments, according to Scheme 3, a compound of formula 10 can be converted to a benzoic acid in the presence of bromine and a base such as sodium hydroxide. An aqueous acid hydrolysis followed by a reductive amination or alkylation as described in Scheme 2 can give the benzoic acid derivatives 17.

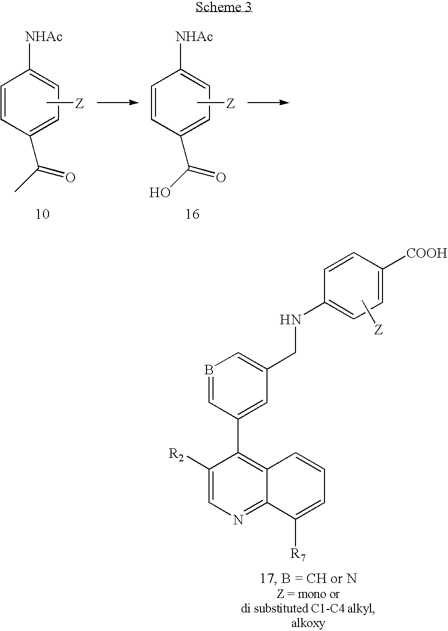

The compounds of formula 23 or 24 can be prepared according to Scheme 4. Nitration of 2-(naphthalen-1-yl)acetic acid (18) can give a mixture of position isomers, and 19 and 20 can be isolated by silica gel chromatography. Hydrogenation in the presence of a palladium catalyst can provide the anilines 21 and 22 which can be converted to the corresponding carboxylic acids 23 and 24 under a standard reductive amination condition.

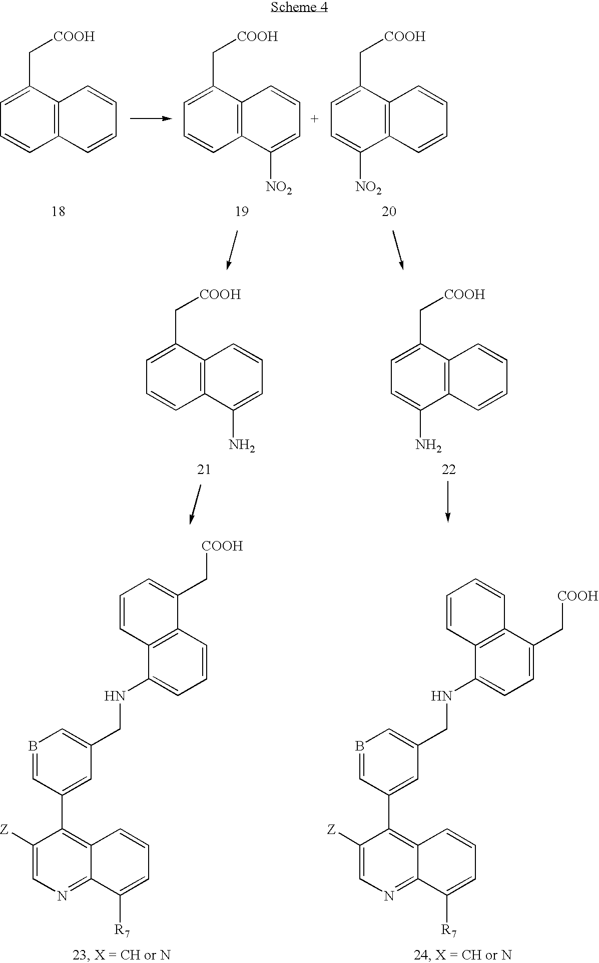

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds, carbon-nitrogen bonds such as amide bonds) wherein bond rotation is restricted about that particular linkage, e.g. restriction resulting from the presence of a ring or double bond. Accordingly, all cis/trans and E/Z isomers and rotational isomers are expressly included in the present invention. The compounds of this invention may also be represented in multiple tautomeric forms, in such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, digluconate, dodecylsulfate, ethanesulfonate, formate, fumarate, glucoheptanoate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts. Salts derived from appropriate bases include alkali metal (e.g., sodium), alkaline earth metal (e.g., magnesium), ammonium and N-(alkyl)₄⁺ salts. This invention also envisions the quaternization of any basic nitrogen-containing groups of the compounds disclosed herein. Water or oil-soluble or dispersible products may be obtained by such quaternization. Salt forms of the compounds of any of the formulae herein can be amino acid salts of carboxy groups (e.g. L-arginine, -lysine, -histidine salts).

The term “pharmaceutically acceptable carrier or adjuvant” refers to a carrier or adjuvant that may be administered to a subject (e.g., a patient), together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

In general, the compounds described herein can be used for treating (e.g., controlling, ameliorating, preventing, delaying the onset of, or reducing the risk of developing) one or more diseases, disorders, conditions or symptoms mediated by LXRs (e.g., cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis).

A disorder or physiological condition that is mediated by LXR refers to a disorder or condition wherein LXR can trigger the onset of the condition, or where inhibition of a particular LXR can affect signaling in such a way so as to treat, control, ameliorate, prevent, delay the onset of, or reduce the risk of developing the disorder or condition. Examples of such disorders include, but are not limited to cardiovascular diseases (e.g., acute coronary syndrome, restenosis), atherosclerosis, atherosclerotic lesions, type I diabetes, type II diabetes, Syndrome X, obesity, lipid disorders (e.g., dyslipidemia, hyperlipidemia, hypertriglyceridemia, hypercholesterolemia, low HDL and high LDL), cognitive disorders (e.g., Alzheimer's disease, dementia), inflammatory diseases (e.g., multiple sclerosis, rheumatoid arthritis, inflammatory bowel disease, Crohn's disease, endometriosis, LPS-induced sepsis, acute contact dermatitis of the ear, chronic atherosclerotic inflammation of the artery wall), celiac, thyroiditis, skin aging (e.g., skin aging is derived from chronological aging, photoaging, steroid-induced skin thinning, or a combination thereof), or connective tissue disease (e.g., osteoarthritis or tendonitis)).

While not wishing to be bound by theory, it is believed that LXR modulators that activate cholesterol efflux (e.g., upregulate ABCA1), but do not substantially increase SREBP-1c expression and triglyceride synthesis in liver, can both reduce atherosclerotic risk and minimize the likelihood of concommitantly increasing serum and hepatic triglyceride levels. Candidate compounds having differential activity for regulating ABCA1 (ABCG1) vs. SREBP-1c can be can be evaluated using conventional pharmacological test procedures, which measure the affinity of a candidate compound to bind to LXR and to upregulate the gene ABCA1.

In some embodiments, LXR ligands can be identified initially in cell-free LXR beta and LXR alpha competition binding assays. LXR ligands can be further characterized by gene expression profiling for tissue selective gene regulation.

In certain embodiments, a compound of formula (I) can have an LXRα/LXRβ binding ratio of from about 10 to about 19; from about 20 to about 27; from about 28 to about 44 (e.g., from about 28 to about 38); or from about 45 to about 50. Examples of such compounds include those described in examples 1-10 and 12-27.

In some embodiments, the compounds described herein have agonist activity for ABCA1 transactivation but do not substantially affect (e.g., inhibit) SREBP-1c gene expression in differentiated THP-1 macrophages. Gene expression analysis in an antagonist mode can be used to further delineate differential regulation of ABCA1 and SREBP-1c gene expression. In certain embodiments, the compounds described herein preferentially antagonize SREBP-1c activation (a marker for genes involved in cholesterol and fatty acid homeostasis) but do not substantially affect (e.g., have relatively minimal or additive effects) on ABCA1 gene expression or genes known to enhance HDL biogenesis (based on a competition assay with known potent synthetic LXR agonists). Cell type or tissue specificity may be further evaluated in additional cell lines, intestinal, CaCo2 or liver, HepG2 and Huh-7 cells where ABCA1 activity is believed to influence net cholesterol absorption and reverse cholesterol transport. The test procedures performed, and results obtained therefrom are described in the Examples section.

In some embodiments, the compounds described herein have agonist activity for ABCA1 and antagonist activity for SREBP-1c (e.g., as determined by gene specific modulation in cell based assays). In certain embodiments, the compounds described herein (in the agonist mode) have at least about 20% efficacy for ABCA1 activation by LXR and do not substantially agonize SREBP-1c (at most about 25% efficacy relative to a reference compound N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838)). In certain embodiments, the compounds described herein (in the antagonist mode) do not substantially antagonize ABCA1 gene expression. While not wishing to be bound by theory, it is believed that there may be an additive effect on ABCA1 gene expression relative to the reference compound at their EC₅₀ concentration. In certain embodiments, the compounds described herein (in the antagonist mode) inhibited agonist-mediated SREBP-1c gene expression in a dose dependent fashion.

In some embodiments, to study the effect of the compounds of formula (I) on skin aging, for example, in a clinical trial, cells can be isolated and RNA prepared and analyzed for the levels of expression of TIMP1, ABCA12, decorin, TNFα, MMP1, MMP3, and/or IL-8. The levels of gene expression (i.e., a gene expression pattern) can be quantified, for example, by Northern blot analysis or RT-PCR, by measuring the amount of protein produced, or by measuring the levels of activity of TIMP1, ABCA12, decorin, TNFα, MMP1, MMP3, and/or IL-8, all by methods known to those of ordinary skill in the art. In this way, the gene expression pattern can serve as a marker, indicative of the physiological response of the cells to the compounds of formula (I). Accordingly, this response state may be determined before, and at various points during, treatment of the individual with the compounds of formula (I).

In one embodiment, expression levels of cytokines and metalloproteases described herein can be used to facilitate design and/or identification of compounds that treat skin aging through an LXR-based mechanism. Accordingly, the invention provides methods (also referred to herein as “screening assays”) for identifying modulators, i.e., LXR modulators, that have a stimulatory or inhibitory effect on, for example, TIMP1, ABCA12, decorin, TNFα, MMP1, MMP3, and/or IL-8 expression.

An exemplary screening assay is a cell-based assay in which a cell that expresses LXR is contacted with a test compound, and the ability of the test compound to modulate TIMP1, ABCA12, decorin, TNFα, MMP1, MMP3, and/or IL-8 expression through an LXR-based mechanism. Determining the ability of the test compound to modulate TIMP1, ABCA12, decorin, TNFα, MMP1, MMP3, and/or IL-8 expression can be accomplished by monitoring, for example, DNA, mRNA, or protein levels, or by measuring the levels of activity of TIMP1, ABCA12, decorin, TNFα, MMP1, MMP3, and/or IL-8, all by methods known to those of ordinary skill in the art. The cell, for example, can be of mammalian origin, e.g., human.

In some embodiments, to study the effect of the compounds of formula (I) on osteoarthritis, for example, in a clinical trial, cells can be isolated and RNA prepared and analyzed for the levels of expression of ApoD and other genes implicated in osteoarthritis (for example, TNFα). The levels of gene expression (i.e., a gene expression pattern) can be quantified by Northern blot analysis or RT-PCR, by measuring the amount of protein produced, or by measuring the levels of activity of ApoD or other genes, all by methods known to those of ordinary skill in the art. In this way, the gene expression pattern can serve as a marker, indicative of the physiological response of the cells to the LXR modulator. Accordingly, this response state may be determined before, and at various points during, treatment of the individual with the LXR modulator.

An exemplary screening assay is a cell-based assay in which a cell that expresses LXR is contacted with a test compound, and the ability of the test compound to modulate ApoD expression and/or aggrecanase activity and/or cytokine elaboration through an LXR-based mechanism. Determining the ability of the test compound to modulate ApoD expression and/or aggrecanase activity and/or cytokine elaboration can be accomplished by monitoring, for example, DNA, mRNA, or protein levels, or by measuring the levels of activity of ApoD, aggrecanase, and/or TNFα, all by methods known to those of ordinary skill in the art. The cell, for example, can be of mammalian origin, e.g., human.

In some embodiments, the compounds described herein can be coadministered with one or more other therapeutic agents. In certain embodiments, the additional agents may be administered separately, as part of a multiple dose regimen, from the compounds of this invention (e.g., sequentially, e.g., on different overlapping schedules with the administration of one or more compounds of formula (I) (including any subgenera or specific compounds thereof)). Alternatively, these agents may be part of a single dosage form, mixed together with the compounds of this invention in a single composition. In still another embodiment, these agents can be given as a separate dose that is administered at about the same time that one or more compounds of formula (I) (including any subgenera or specific compounds thereof) are administered (e.g., simultaneously with the administration of one or more compounds of formula (I) (including any subgenera or specific compounds thereof)). When the compositions of this invention include a combination of a compound of the formulae described herein and one or more additional therapeutic or prophylactic agents, both the compound and the additional agent can be present at dosage levels of between about 1 to 100%, and more preferably between about 5 to 95% of the dosage normally administered in a monotherapy regimen.

The compounds and compositions described herein can, for example, be administered orally, parenterally (e.g., subcutaneously, intracutaneously, intravenously, intramuscularly, intraarticularly, intraarterially, intrasynovially, intrasternally, intrathecally, intralesionally and by intracranial injection or infusion techniques), by inhalation spray, topically, rectally, nasally, buccally, vaginally, via an implanted reservoir, by injection, subdermally, intraperitoneally, transmucosally, or in an ophthalmic preparation, with a dosage ranging from about 0.01 mg/Kg to about 1000 mg/Kg, (e.g., from about 0.01 to about 100 mg/kg, from about 0.1 to about 100 mg/Kg, from about 1 to about 100 mg/Kg, from about 1 to about 10 mg/kg) every 4 to 120 hours, or according to the requirements of the particular drug. The interrelationship of dosages for animals and humans (based on milligrams per meter squared of body surface) is described by Freireich et al., Cancer Chemother. Rep. 50, 219 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or administration by injection. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

The compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the compositions of this invention is useful when the desired treatment involves areas or organs readily accessible by topical application. For application topically to the skin, the composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The compositions of this invention may also be topically applied to the lower intestinal tract by rectal suppository formulation or in a suitable enema formulation.

In some embodiments, topical administration of the compounds and compositions described herein may be presented in the form of an aerosol, a semi-solid pharmaceutical composition, a powder, or a solution. By the term “a semi-solid composition” is meant an ointment, cream, salve, jelly, or other pharmaceutical composition of substantially similar consistency suitable for application to the skin. Examples of semi-solid compositions are given in Chapter 17 of The Theory and Practice of Industrial Pharmacy, Lachman, Lieberman and Kanig, published by Lea and Febiger (1970) and in Chapter 67 of Remington's Pharmaceutical Sciences, 15th Edition (1975) published by Mack Publishing Company.

Topically-transdermal patches are also included in this invention. Also within the invention is a patch to deliver active chemotherapeutic combinations herein. A patch includes a material layer (e.g., polymeric, cloth, gauze, bandage) and the compound of the formulae herein as delineated herein. One side of the material layer can have a protective layer adhered to it to resist passage of the compounds or compositions. The patch can additionally include an adhesive to hold the patch in place on a subject. An adhesive is a composition, including those of either natural or synthetic origin, that when contacted with the skin of a subject, temporarily adheres to the skin. It can be water resistant. The adhesive can be placed on the patch to hold it in contact with the skin of the subject for an extended period of time. The adhesive can be made of a tackiness, or adhesive strength, such that it holds the device in place subject to incidental contact, however, upon an affirmative act (e.g., ripping, peeling, or other intentional removal) the adhesive gives way to the external pressure placed on the device or the adhesive itself, and allows for breaking of the adhesion contact. The adhesive can be pressure sensitive, that is, it can allow for positioning of the adhesive (and the device to be adhered to the skin) against the skin by the application of pressure (e.g., pushing, rubbing) on the adhesive or device.

The compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

A composition having the compound of the formulae herein and an additional agent (e.g., a therapeutic agent) can be administered using any of the routes of administration described herein. In some embodiments, a composition having the compound of the formulae herein and an additional agent (e.g., a therapeutic agent) can be administered using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). Negrin et al., Biomaterials, 22(6):563 (2001). Timed-release technology involving alternate delivery methods can also be used in this invention. For example, timed-release formulations based on polymer technologies, sustained-release techniques and encapsulation techniques (e.g., polymeric, liposomal) can also be used for delivery of the compounds and compositions delineated herein.

The invention will be further described in the following examples. It should be understood that these examples are for illustrative purposes only and are not to be construed as limiting this invention in any manner.

### EXAMPLES

The following describes the preparation of representative compounds of this invention. Compounds described as homogeneous are determined to be of 90% or greater purity (exclusive of enantiomers) by analytical reverse phase chromatographic analysis with 254 nM UV detection. Melting points are reported as uncorrected in degrees centigrade. Mass spectral data is reported as the mass-to-charge ratio, m/z; and for high resolution mass spectral data, the calculated and experimentally found masses, [M+H]⁺, for the neutral formulae M are reported. All reactions are stirred and run under a nitrogen atmosphere unless otherwise noted.

The following describes the preparation of representative compounds of this invention. Compounds described as homogeneous are determined to be of 90% or greater purity (exclusive of enantiomers) by analytical reverse phase chromatographic analysis with 254 nM UV detection. Melting points are reported as uncorrected in degrees centigrade. Mass spectral data is reported as the mass-to-charge ratio, m/z; and for high-resolution mass spectral data, the calculated and experimentally found masses, [M+H]⁺, for the neutral formulae M are reported. All reactions are stirred and run under a nitrogen atmosphere unless otherwise noted.

### Example 1

### [5-({3-[8-(Trifluoromethyl)quinolin-4-yl]benzyl}amino)-1-naphthyl]acetic acid

Step 1: A mixture of nitric acid (50 mL, 70%) and 2-(naphthalen-1-yl)acetic acid (4.3 g, 23.1 mmol) was stirred at room temperature for 1 hour. ˜200 mL of water was added into the reaction mixture and the solid was collected. The mixture was separated into (5-nitro-1-naphthyl)acetic acid (pale yellow solid, 0.55 g, 10%) and 2-(4-nitronaphthalen-1-yl)acetic acid (pale yellow solid, 0.5 g, 9%);

Step 2: (5-Amino-1-naphthyl)acetic acid was prepared from (5-nitro-1-naphthyl)acetic acid by hydrogenation (Pd/C, 20 psi of H₂) for 1 hour (70%); MS (ES) m/z 202;

Step 3: A solution of 4-chloro-8-(trifluoromethyl)quinoline (1.65 g, 5.0 mmol), 3-formylphenyl boronic acid (15.0 g, 10 mmol), Pd(PPh₃)₄ (0.58 g, 0.5 mmol) in 2M aqueous sodium carbonate (10 mL), toluene (20 mL) and ethanol (5 mL) was heated to reflux. After 1 hr, the reaction was cooled, partitioned between water and EtOAc, dried over MgSO₄ and concentrated in vacuo, and the residue was purified by chromatography eluting with ethyl acetate/hexanes to give 3-(8-(trifluoromethyl)quinolin-4-yl)benzaldehyde (1.31 g, 86%) as a gummy solid; MS (ESI) m/z 301.9 (M+H)⁺.

Step 4: 3-(8-(Trifluoromethyl)quinolin-4-yl)benzaldehyde (0.05 g, 0.17 mmol) and (5-amino-1-naphthyl)acetic acid (0.05 g, 0.23 mmol) were mixed in DMF (2.5 mL) and then treated with NaBH(OAc)₃ (0.3 g, 1.4 mml) and acetic acid (2.5 mL). After stirring at room temperature under a N₂ atmosphere for 2 h the mixture was quenched with water and then extracted with ethyl acetate. The organic residue was purified by reverse phase HPLC to provide the title compound (0.025 g, 30%) as a solid; MS (EI) m/z 486.9.

### Example 2

### {4-[({5-[3-benzyl-8-(trifluoromethyl)quinolin-4-yl]pyridin-3-yl}methyl)amino]-2,5-dimethylphenyl}acetic acid

Step 1: A solution of 3-benzyl-4-bromo-8-(trifluoromethyl)quinoline (1.1 g, 3.0 mmol), 5-formylpyridine-3-boronic acid pinacol ester (1.0 g, 4.29 mmol), Pd(PPh₃)₄ (0.58 g, 0.5 mmol) in 2M aqueous sodium carbonate (5 mL), toluene (20 mL) and ethanol (5 mL) was heated to reflux. After 2 hr, the reaction was cooled, filtered through celite, partitioned between water and EtOAc, dried over MgSO₄ and concentrated in vacuo. The residue was purified by chromatography eluting with ethyl acetate/hexanes to give 5-[3-benzyl-8-(trifluoromethyl)quinolin-4-yl]nicotinaldehyde (1.2 g, 31%) as a gummy solid. MS (ESI) m/z 392.8 (M+H)⁺;

Step 2: Acetic chloride (15.6 g, 200 mmol) was added slowly to a stirred solution of 2,5-dimethylaniline (24.2 g, 200 mmol), pyridine (40 mL) and DCM (200 mL) at 0-5° C. and the mixture was stirred at 0-5° C. for 1 hr. After warming to room temperature, the reaction was partitioned between water and EtOAc, dried over MgSO₄ and concentrated in vacuo. The residue was purified by recrystallization from EtOAc/hexanes to give N-(2,5-dimethylphenyl)acetamide (20 g, 61%) as a pale brown solid

Step 3: A mixture of N-(2,5-dimethylphenyl)acetamide (16.0 g, 10 mmol), acetic chloride (15.6 g, 400 mmol), AlCl₃ (40 g), and carbon disulfide (200 mL) was heated to reflux. After 2 hr, the reaction was cooled and the solvent was decanted and the residue was treated with ice. The solid was collected and dried to give N-(4-acetyl-2,5-dimethylphenyl)acetamide as an off-white solid in quantitative yield; MS (ES) m/z 206.2;

Step 4: A mixture of N-(4-acetyl-2,5-dimethylphenyl)acetamide (20.5 g, 100 mmol), morpholine (11.7 g, 134 mmol), sulfur (3.2 g, 100 mmol) was heated at 110° C. for 16 hr. The reaction was cooled, partitioned between water and DCM, dried over MgSO₄ and concentrated in vacuo. The residue was concentrated to give N-[2,5-dimethyl-4-(2-morpholin-4-yl-2-thioxoethyl)phenyl]acetamide (21 g, 68%) as an off white solid; MS (ES) m/z 307.2;

Step 5: A mixture of N-[2,5-dimethyl-4-(2-morpholin-4-yl-2-thioxoethyl)phenyl]acetamide (21.0 g, 68.4 mmol) in 6 N aqueous HCl was heated to reflux. After 2 hr the reaction was cooled, pH was adjusted to 5 by concentrated ammonium hydroxide, and the solid was collected and dried to give (4-amino-2,5-dimethylphenyl)acetic acid (8.2 g, 66%) as an off white solid (MS (ESI) m/z 180;

Step 6: The title compound was prepared from 5-[3-benzyl-8-(trifluoromethyl)quinolin-4-yl]nicotinaldehyde and (4-amino-2,5-dimethylphenyl)acetic acid following the procedure of Example 1, Step 4 as a pale yellow solid; MS (ES) m/z 555.9.

### Example 3

### {5-[({5-[3-Benzyl-8-(trifluoromethyl)quinolin-4-yl]pyridin-3-yl}methyl)amino]-1-naphthyl}acetic acid

The title compound was prepared from 5-[3-benzyl-8-(trifluoromethyl)quinolin-4-yl]nicotinaldehyde and (5-amino-1-naphthyl)acetic acid following the procedure of Example 1, Step 4 as a pale yellow solid; MS (ES) m/z 578.0.

### Example 4

### [2,5-Dimethyl-4-({3-[8-(trifluoromethyl)quinolin-4-yl]benzyl}amino)phenyl]acetic acid

Step 1: 3-[8-(Trifluoromethyl)quinolin-4-yl]benzaldehyde was prepared from 4-bromo-8-(trifluoromethyl)quinoline and 3-formylphenylboronic acid following the procedure of Example 1 Step 3 as a white solid; MS (ES) m/z 301.9;

Step 2: The title compound was prepared from 3-(8-(trifluoromethyl)quinolin-4-yl)benzaldehyde and (5-amino-1-naphthyl)acetic acid following the procedure of Example 1 Step 4 as a pale yellow solid; MS (ES) m/z 464.9.

### Example 5

### [5-({3-[8-(Trifluoromethyl)quinolin-4-yl]benzyl}oxy)-1-naphthyl]acetic acid

Step 1: {3-[8-(Trifluoromethyl)quinolin-4-yl]phenyl}methanol was prepared from 3-benzyl-4-chloro-8-(trifluoromethyl)quinoline and 3-(hydroxymethyl)phenylboronic acid following the procedure of Example 1 Step 3 as a gummy solid; MS (ESI) m/z 303 (M+H)+;

Step 2: To a mixture of 5-hydroxy-1-naphthaleneacetic acid methyl ester (WO 2003011862) (0.07 g, 0.32 mmol), PPh₃ on polymer (0.5 g, 1.5 mmol) and {3-[8-(trifluoromethyl)quinolin-4-yl]phenyl}methanol (0.05 g, 0.17 mmol) in DCM (15 ml) was added DEAD (0.21 g, 1.0 mmol) drop wise. After 2 hr, the reaction was concentrated and purified by column chromatography (eluent EtOAc/Hexane) to give a gum. The gum was dissolved in THF/MeOH/water (2:1:1, 15 ml) and solid LiOH (100 mg) and added. After 2 hr, the reaction was acidified with acetic acid and the concentrated residue was purified by reverse phase HPLC to give the title compound as a white solid (0.045 g, 55%); MS (ES) m/z 486.1.

### Example 6

### 6-[({5-[3-Benzyl-8-(trifluoromethyl)quinolin-4-yl]pyridin-3-yl}methyl)amino]-2-naphthoic acid

The title compound was prepared from 5-[3-benzyl-8-(trifluoromethyl)quinolin-4-yl]nicotinaldehyde and 6-amino-2-naphthoic acid following the procedure of Example 1 Step 4 as a solid; MS (ES) m/z 564.0.

### Example 7

### [2,5-Dimethyl-4-({3-[3-methyl-8-(trifluoromethyl)quinolin-4-yl]-benzyl}amino)phenyl]acetic acid

Step 1: 3-[3-Methyl-8-(trifluoromethyl)quinolin-4-yl]benzaldehyde was prepared from 4-bromo-3-methyl-8-(trifluoromethyl)quinoline and 3-formylphenylboronic acid following the procedure of Example 1 Step 3 as a white solid; MS (EI) m/z 315;

Step 2: The title compound was prepared from 3-[3-methyl-8-(trifluoromethyl)quinolin-4-yl]benzaldehyde and (4-amino-2,5-dimethylphenyl)acetic acid following the procedure of Example 1 Step 4 as a solid; MS (ES) m/z 478.8;

### Example 8

### [5-({3-[3-Benzyl-8-(trifluoromethyl)quinolin-4-yl]benzyl}amino)-1-naphthyl]acetic acid

Step 1: 3-[3-Benzyl-8-(trifluoromethyl)quinolin-4-yl]benzaldehyde was prepared from 3-benzyl-4-bromo-8-(trifluoromethyl)quinoline and 3-formylphenylboronic acid following the procedure of Example 1 Step 3 as a white solid; MS (ES) m/z 392.1;

Step 2: The title compound was prepared from 3-[3-benzyl-8-(trifluoromethyl)quinolin-4-yl]benzaldehyde and 2-(5-aminonaphthalen-1-yl)acetic acid following the procedure of Example 1 Step 4 as a solid; MS (ES) m/z 576.8.

### Example 9

### 5-({3-[3-Methyl-8-(trifluoromethyl)quinolin-4-yl]benzyl}oxy)-1-naphthyl]acetic acid

Step 1: {3-[3-Methyl-8-(trifluoromethyl)quinolin-4-yl]phenyl}methanol was prepared from 3-[3-methyl-8-(trifluoromethyl)quinolin-4-yl]benzaldehyde by BaBH₄ reduction; MS (ES) m/z 318.0;

Step 2: The title compound was prepared from {3-[3-methyl-8-(trifluoromethyl)quinolin-4-yl]phenyl}methanol and 5-hydroxy-1-naphthaleneacetic acid methyl ester (WO 2003011862) following the procedure of Example 5 Step 2 as a solid; MS (ES) m/z 499.9.

### Example 10

### {4-[({5-[3-Benzyl-8-(trifluoromethyl)quinolin-4-yl]pyridin-3-yl}methyl)amino]-2,3-dimethylphenyl}acetic acid

Step 1: (4-Amino-2,3-dimethylphenyl)acetic acid was prepared from 2,3-dimethylaniline following the procedure of Example 2;

Step 2: The title compound was prepared from 5-[3-benzyl-8-(trifluoromethyl)quinolin-4-yl]nicotinaldehyde and (4-amino-2,3-dimethylphenyl)acetic acid following the procedure of Example 1 Step 4 as a pale yellow solid; MS (ES) m/z 555.9.

### Example 11

### N-[4-({3-[3-Benzyl-8-(trifluoromethyl)quinolin-4-yl]phenoxy}methyl)benzoyl]methionine

To 3-((3-(3-benzyl-8-(trifluoromethyl)quinolin-4-yl)phenoxy)methyl)benzoic acid (50 mg, 0.097 mmol) in CH₂Cl₂ (6 ml) at room temperature under a nitrogen atmosphere was added in the following order: methyl 2-amino-4-(methylthio)butanoate (17.5 mg, 0.102 mmol), 1-[3-(dimethylamino)propyl]-3-ethylcarbodiimide hydrochloride (22 mg, 0.117 mmol), 1-hydroxybenzotriazole hydrate (16 mg, 0.117 mmol) and N-methylmorpholine (39 mg, 0.895 mmol). The resulting yellow mixture was stirred 15 hrs. The reaction was quenched with water (0.5 ml), filtered and the filtrate concentrated in vacuo to a yellow paste. This same yellow paste was subjected to reversed phased chromatography (AcCN:H₂O) to afforded a yellow powder methyl 2-(3-((3-(3-benzyl-8-(trifluoromethyl)quinolin-4-yl)phenoxy)methyl)benzamido)-4-(methylthio)butanoate (59 mg, 92% yield). To this same ester was dissolved up in THF:H₂O/4:1 (5 ml) was added lithium hydroxide (3.9 mg, 3.895 mmol) and heated at 50° C. for 3 hrs. The reaction was cooled to room temperature upon which was quenched with aqueous 1N HCl (pH<2) and concentrated in vacuo to afforded the crude carboxylic acid as an off white powder. This same crude acid was subjected to reversed phased chromatography (AcCN:H₂O) to afford the title compound as a white powder (58 mg, 89% yield); MS (ES) m/z 645.2.

### Example 12

### 4-({3-[3-Benzyl-8-(trifluoromethyl)quinolin-4-yl]benzyl}amino)-2,5-dimethylbenzoic acid

Step 1: 4-(Acetylamino)-2,5-dimethylbenzoic acid was prepared by treating N-(4-acetyl-2,5-dimethylphenyl)acetamide with bromine and NaOH; MS (ES) m/z 208.1;

Step 2: 4-Amino-2,5-dimethylbenzoic acid was prepared by treating 4-(acetylamino)-2,5-dimethylbenzoic acid with HCl as described in Example 2; MS (ES) m/z 166.0;

Step 3: The title compound was prepared from 3-[3-benzyl-8-(trifluoromethyl)quinolin-4-yl]benzaldehyde and 4-amino-2,5-dimethylbenzoic acid following the procedure of Example 1 Step 4 as a solid; MS (ES) m/z 541.0.

By procedures similar to those in the preceding examples, the following Examples 13 to 120 were prepared.

### Example 13

### [5-({3-[3-methyl-8-(trifluoromethyl)quinolin-4-yl]benzyl}amino)-1-naphthyl]acetic acid

MS (ES) m/z 500.9. Prepared in a manner similar to Example 1.

### Example 14

### 4-[({5-[3-benzyl-8-(trifluoromethyl)quinolin-4-yl]pyridin-3-yl}methyl)amino]-2,5-dimethylbenzoic acid

MS (ES) m/z 542.1. Prepared in a manner similar to Example 12.

### Example 15

### 6-({3-[3-methyl-8-(trifluoromethyl)quinolin-4-yl]benzyl}amino)-2-naphthoic acid

MS (ES) m/z 487.1. Prepared in a manner similar to Example 12.

### Example 16

### [2,3-dimethyl-4-({3-[3-methyl-8-(trifluoromethyl)quinolin-4-yl]benzyl}amino)phenyl]acetic acid

MS (ES) m/z 478.9. Prepared in a manner similar to Example 1.

### Example 17

### [4-({5-[3-benzyl-8-(trifluoromethyl)quinolin-4-yl]pyridin-3-yl}methoxy)-2,5-dimethylphenyl]acetic acid

MS (ES) m/z 557.1. Prepared in a manner similar to Example 5.

### Example 18

### 4-({3-[3-benzyl-8-(trifluoromethyl)quinolin-4-yl]benzyl}amino)-5-chloro-2-methoxybenzoic acid

MS (ES) m/z 577.0. Prepared in a manner similar to Example 12.

### Example 19

### [5-({3-[3-benzyl-8-(trifluoromethyl)quinolin-4-yl]benzyl}oxy)-3,4-dihydronaphthalen-1-yl]acetic acid

MS (ES) m/z 579.9. Prepared in a manner similar to Example 5.

### Example 20

### [4-({3-[3-benzyl-8-(trifluoromethyl)quinolin-4-yl]benzyl}amino)-2,3-dimethylphenyl]acetic acid

MS (ES) m/z 555.2; Prepared in a manner similar to Example 1.

### Example 21

### [5-({3-[3-benzyl-8-(trifluoromethyl)quinolin-4-yl]benzyl}oxy)-1-naphthyl]acetic acid

MS (ES) m/z 578.1; Prepared in a manner similar to Example 5

### Example 22

### [2,3-dimethyl-4-({3-[8-(trifluoromethyl)quinolin-4-yl]benzyl}amino)phenyl]acetic acid

MS (ES) m/z 464.9; Prepared in a manner similar to Example 1.

### Example 23

### 6-({3-[3-benzyl-8-(trifluoromethyl)quinolin-4-yl]benzyl}amino)-2-naphthoic acid

MS (ES) m/z 563.1; Prepared in a manner similar to Example 12.

### Example 24

### [4-({3-[3-benzyl-8-(trifluoromethyl)quinolin-4-yl]benzyl}amino)-2,5-dimethylphenyl]acetic acid

MS (ES) m/z 555.2; Prepared in a manner similar to Example 4.

### Example 25

### 2,5-dimethyl-4-({3-[3-methyl-8-(trifluoromethyl)quinolin-4-yl]benzyl}amino)benzoic acid

MS (ES) m/z 465.0; Prepared in a manner similar to Example 12.

### Example 26

### 4-({3-[3-benzyl-8-(trifluoromethyl)quinolin-4-yl]benzyl}oxy)-3,5-dimethylbenzoic acid

MS (ES) m/z 541.9; Prepared in a manner similar to Example 5.

### Example 27

### 3-[4-({3-[3-benzyl-8-(trifluoromethyl)quinolin-4-yl]benzyl}amino)phenyl]propanoic acid

MS (ES) m/z 538.8; Prepared in a manner similar to Example 1.

### Example 28

### 4-({3-[3-benzyl-8-(trifluoromethyl)quinolin-4-yl]phenoxy}methyl)-2-methoxybenzoic acid

MS (ES) m/z 543.8; Prepared in a manner similar to Example 5.

### Example 29

### [4-({3-[3-benzyl-8-(trifluoromethyl)quinolin-4-yl]benzyl}amino)-1-naphthyl]acetic acid

MS (ES) m/z 577.2; Prepared in a manner similar to Example 1.

### Example 30

### [3-({3-[3-benzyl-8-(trifluoromethyl)quinolin-4-yl]benzyl}amino)phenyl]acetic acid

MS (ES) m/z 525.1; Prepared in a manner similar to Example 1.

### Example 31

### 5-[({5-[3-benzyl-8-(trifluoromethyl)quinolin-4-yl]pyridin-3-yl}methyl)amino]-1-naphthoic acid

MS (ES) m/z 564.0; Prepared in a manner similar to Example 12.

### Example 32

### {4-[({2-[3-benzyl-8-(trifluoromethyl)quinolin-4-yl]-3-thienyl}methyl)amino]-2,5-dimethylphenyl}acetic acid

MS (ES) m/z 560.8; Prepared in a manner similar to Example 1.

### Example 33

### 2-({3-[3-benzyl-8-(trifluoromethyl)quinolin-4-yl]benzyl}amino)-4-methylbenzoic acid

MS (ES) m/z 524.8; Prepared in a manner similar to Example 12.

### Example 34

### [4-({3-[3-benzyl-8-(trifluoromethyl)quinolin-4-yl]benzyl}oxy)-2,5-dimethylphenyl]acetic acid

MS (ES) m/z 553.9; Prepared in a manner similar to Example 5.

### Example 35

### [2-({3-[3-benzyl-8-(trifluoromethyl)quinolin-4-yl]benzyl}amino)-3,4-dimethylphenyl]acetic acid

MS (ES) m/z 555.2; Prepared in a manner similar to Example 1.

### Example 36

### {4-[({5-[3-benzyl-8-(trifluoromethyl)quinolin-4-yl]pyridin-3-yl}methyl)amino]phenyl}acetic acid

MS (ES) m/z 528.1; Prepared in a manner similar to Example 2.

### Example 37

### [5-({3-[3-benzyl-8-(trifluoromethyl)quinolin-4-yl]benzyl}oxy)-1,2,3,4-tetrahydronaphthalen-1-yl]acetic acid

MS (ES) m/z 581.9; Prepared in a manner similar to Example 5.

### Example 38

### [4-({3-[3-benzyl-8-(trifluoromethyl)quinolin-4-yl]benzyl}oxy)-2,3-dihydro-1H-inden-1-yl]acetic acid

MS (ES) m/z 567.9; Prepared in a manner similar to Example 5.

### Example 39

### 6-({3-[3-benzyl-8-(trifluoromethyl)quinolin-4-yl]benzyl}oxy)-2-naphthoic acid

MS (ES) m/z 564.0; Prepared in a manner similar to Example 5.

### Example 40

### 1-[3-({3-[3-benzyl-8-(trifluoromethyl)quinolin-4-yl]phenoxy}methyl)benzoyl]piperidine-4-carboxylic acid

MS (ES) m/z 625.2; Prepared in a manner similar to Example 11.

### Example 41

### 2-({3-[3-benzyl-8-(trifluoromethyl)quinolin-4-yl]benzyl}amino)-4-fluorobenzoic acid

MS (ES) m/z 531.3; Prepared in a manner similar to Example 12.

### Example 42

### [4-({3-[3-benzyl-8-(trifluoromethyl)quinolin-4-yl]benzyl}amino)phenyl]acetic acid

MS (ES) m/z 527.4; Prepared in a manner similar to Example 1.

### Example 43

### 3-[3-({3-[3-benzyl-8-(trifluoromethyl)quinolin-4-yl]benzyl}amino)phenyl]propanoic acid

MS (ES) m/z 541.3; Prepared in a manner similar to Example 1.

### Example 44

### 7-({3-[3-benzyl-8-(trifluoromethyl)quinolin-4-yl]benzyl}amino)-1-methyl-1H-indole-3-carboxylic acid

MS (ESI) m/z 566; Prepared in a manner similar to Example 12.

### Example 45

### 4-({3-[3-benzyl-8-(trifluoromethyl)quinolin-4-yl]benzyl}oxy)-3-methoxybenzoic acid

MS (ES) m/z 543.8; Prepared in a manner similar to Example 5.

### Example 46

### 6-({3-[8-(trifluoromethyl)quinolin-4-yl]benzyl}amino)-2-naphthoic acid

MS (ES) m/z 473.1; Prepared in a manner similar to Example 12.

### Example 47

### {4-[({5-[3-benzyl-8-(trifluoromethyl)quinolin-4-yl]-2-thienyl}methyl)amino]-2,5-dimethylphenyl}acetic acid

MS (ES) m/z 560.9; Prepared in a manner similar to Example 1.

### Example 48

### 5-({3-[8-(trifluoromethyl)quinolin-4-yl]benzyl}amino)-1-naphthoic acid

MS (ES) m/z 473.0; Prepared in a manner similar to Example 12.

### Example 49

### 2-({3-[3-benzyl-8-(trifluoromethyl)quinolin-4-yl]benzyl}amino)-6-(trifluoromethyl)benzoic acid

MS (ES) m/z 581.3; Prepared in a manner similar to Example 12.

### Example 50

### [4-({3-[3-benzyl-8-(trifluoromethyl)quinolin-4-yl]benzyl}amino)-2-chlorophenyl]acetic acid

MS (ES) m/z 560.8; Prepared in a manner similar to Example 2.

### Example 51

### 3-({3-[3-benzyl-8-(trifluoromethyl)quinolin-4-yl]benzyl}amino)benzoic acid

MS (ES) m/z 513.3; Prepared in a manner similar to Example 12.

### Example 52

### 3-({3-[3-benzyl-8-(trifluoromethyl)quinolin-4-yl]benzyl}amino)-4-fluorobenzoic acid

MS (ES) m/z 530.7; Prepared in a manner similar to Example 12.

### Example 53

### 4-({3-[3-benzyl-8-(trifluoromethyl)quinolin-4-yl]benzyl}amino)-3-methylbenzoic acid

MS (ES) m/z 526.8; Prepared in a manner similar to Example 12.

### Example 54

### 1-[4-({3-[3-benzyl-8-(trifluoromethyl)quinolin-4-yl]phenoxy}methyl)benzoyl]proline

MS (ES) m/z 611.2; Prepared in a manner similar to Example 11.

### Example 55

### 2-({3-[3-benzyl-8-(trifluoromethyl)quinolin-4-yl]benzyl}amino)-6-methoxybenzoic acid

MS (ES) m/z 540.7; Prepared in a manner similar to Example 12.

### Example 56

### 5-({3-[3-benzyl-8-(trifluoromethyl)quinolin-4-yl]benzyl}amino)-1-naphthoic acid

MS (ES) m/z 563.1; Prepared in a manner similar to Example 12.

### Example 57

### [6-({3-[3-benzyl-8-(trifluoromethyl)quinolin-4-yl]benzyl}oxy)-1-naphthyl]acetic acid

MS (ES) m/z 578.0; Prepared in a manner similar to Example 5.

### Example 58

### 2-({3-[3-benzyl-8-(trifluoromethyl)quinolin-4-yl]benzyl}amino)-6-fluorobenzoic acid

MS (ES) m/z 531.2; Prepared in a manner similar to Example 12.

### Example 59

### 1-[3-({3-[3-benzyl-8-(trifluoromethyl)quinolin-4-yl]phenoxy}methyl)benzoyl]proline

MS (ES) m/z 611.2; Prepared in a manner similar to Example 11.

### Example 60

### 1-[4-({3-[3-benzyl-8-(trifluoromethyl)quinolin-4-yl]phenoxy}methyl)benzoyl]piperidine-4-carboxylic acid

MS (ES) m/z 625.2; Prepared in a manner similar to Example 11.

### Example 61

### N-[4-({3-[3-benzyl-8-(trifluoromethyl)quinolin-4-yl]phenoxy}methyl)benzoyl]histidine

MS (ES) m/z 648.7; Prepared in a manner similar to Example 11.

### Example 62

### [3-({3-[3-benzyl-8-(trifluoromethyl)quinolin-4-yl]benzyl}oxy)-4-methylphenyl]acetic acid

MS (ES) m/z 541.9; Prepared in a manner similar to Example 5.

### Example 63

### 5-({3-[3-benzyl-8-(trifluoromethyl)quinolin-4-yl]benzyl}amino)-1-methyl-1H-indole-2-carboxylic acid

MS (ES) m/z 565.8; Prepared in a manner similar to Example 12.

### Example 64

### {4-[({4-[3-benzyl-8-(trifluoromethyl)quinolin-4-yl]-3-thienyl}methyl)amino]-2,5-dimethylphenyl}acetic acid

MS (ES) m/z 560.8; Prepared in a manner similar to Example 1.

### Example 65

### [4-({3-[8-(trifluoromethyl)quinolin-4-yl]benzyl}amino)-1-naphthyl]acetic acid

MS (ES) m/z 486.9; Prepared in a manner similar to Example 4.

### Example 66

### 2-[5-({3-[8-(trifluoromethyl)quinolin-4-yl]benzyl}oxy)-1-naphthyl]pent-4-ynoic acid

MS (ES) m/z 525.9; Prepared in a manner similar to Example 5.

### Example 67

### 4-({3-[3-benzyl-8-(trifluoromethyl)quinolin-4-yl]benzyl}oxy)-3,5-dichlorobenzoic acid

MS (ES) m/z 579.6; Prepared in a manner similar to Example 5.

### Example 68

### [6-({3-[8-(trifluoromethyl)quinolin-4-yl]benzyl}oxy)-1-naphthyl]acetic acid

MS (ES) m/z 488.0; Prepared in a manner similar to Example 5.

### Example 69

### [5-({3-[3-benzyl-8-(trifluoromethyl)quinolin-4-yl]benzyl}oxy)-2-naphthyl]acetic acid

MS (ES) m/z 576.0; Prepared in a manner similar to Example 5.

### Example 70

### {4-[({4-[3-benzyl-8-(trifluoromethyl)quinolin-4-yl]-2-thienyl}methyl)amino]-2,5-dimethylphenyl}acetic acid

MS (ES) m/z 560.8; Prepared in a manner similar to Example 4.

### Example 71

### 2-({3-[3-benzyl-8-(trifluoromethyl)quinolin-4-yl]benzyl}amino)-5-methylbenzoic acid

MS (ES) m/z 526.6; Prepared in a manner similar to Example 12.

### Example 72

### N-[3-({3-[3-benzyl-8-(trifluoromethyl)quinolin-4-yl]phenoxy}methyl)benzoyl]histidine

MS (ES) m/z 651.1; Prepared in a manner similar to Example 11.

### Example 73

### 2-chloro-4-({3-[3-methyl-8-(trifluoromethyl)quinolin-4-yl]benzyl}amino)benzoic acid

MS (ES) m/z 470.8; Prepared in a manner similar to Example 12.

### Example 74

### [4-({3-[3-benzyl-8-(trifluoromethyl)quinolin-4-yl]phenoxy}methyl)-2,5-dimethylphenyl]acetic acid

MS (ES) m/z 556.2; Prepared in a manner similar to Example 5.

### Example 75

### 4-({3-[3-benzyl-8-(trifluoromethyl)quinolin-4-yl]benzyl}amino)-2-methylbenzoic acid

MS (ES) m/z 527.0; Prepared in a manner similar to Example 12.

### Example 76

### N-[3-({3-[3-benzyl-8-(trifluoromethyl)quinolin-4-yl]phenoxy}methyl)benzoyl]methionine

MS (ES) m/z 645.2; Prepared in a manner similar to Example 11.

### Example 77

### 7-({3-[3-benzyl-8-(trifluoromethyl)quinolin-4-yl]benzyl}amino)-1H-indole-2-carboxylic acid

MS (ES) m/z 551.7; Prepared in a manner similar to Example 12.

### Example 78

### 3-({3-[3-benzyl-8-(trifluoromethyl)quinolin-4-yl]benzyl}amino)-2-naphthoic acid

MS (ES) m/z 562.8; Prepared in a manner similar to Example 12.

### Example 79

### 5-({3-[3-benzyl-8-(trifluoromethyl)quinolin-4-yl]benzyl}amino)-1-methyl-1H-indole-3-carboxylic acid

MS (ES) m/z 565.9; Prepared in a manner similar to Example 12.

### Example 80

### 5-({3-[3-methyl-8-(trifluoromethyl)quinolin-4-yl]benzyl}amino)-1-naphthoic acid

MS (ES) m/z 487.0; Prepared in a manner similar to Example 12.

### Example 81

### N-[4-({3-[3-benzyl-8-(trifluoromethyl)quinolin-4-yl]phenoxy}methyl)benzoyl]leucine

MS (ESI) m/z 627; Prepared in a manner similar to Example 11.

### Example 82

### 5-({3-[3-benzyl-8-(trifluoromethyl)quinolin-4-yl]benzyl}amino)-2-chlorobenzoic acid

MS (ES) m/z 544.7; Prepared in a manner similar to Example 12.

### Example 83

### 7-({3-[3-benzyl-8-(trifluoromethyl)quinolin-4-yl]benzyl}amino)-1H-indole-3-carboxylic acid

MS (ES) m/z 551.8; Prepared in a manner similar to Example 12.

### Example 84

### 4-({3-[3-benzyl-8-(trifluoromethyl)quinolin-4-yl]benzyl}oxy)-3-fluorobenzoic acid

MS (ES) m/z 529.8; Prepared in a manner similar to Example 5.

### Example 85

### 4-({3-[3-benzyl-8-(trifluoromethyl)quinolin-4-yl]benzyl}oxy)-3-chlorobenzoic acid

MS (ES) m/z 547.8; Prepared in a manner similar to Example 5.

### Example 86

### [7-({3-[3-benzyl-8-(trifluoromethyl)quinolin-4-yl]benzyl}oxy)-1-naphthyl]acetic acid

MS (ES) m/z 578.0; Prepared in a manner similar to Example 5.

### Example 87

### 4-({3-[3-benzyl-8-(trifluoromethyl)quinolin-4-yl]benzyl}amino)-2-chlorobenzoic acid

MS (ES) m/z 544.6; Prepared in a manner similar to Example 12.

### Example 88

### [3,4-dimethyl-5-({3-[3-methyl-8-(trifluoromethyl)quinolin-4-yl]benzyl}amino)phenyl]acetic acid

MS (ES) m/z 478.9; Prepared in a manner similar to Example 2.

### Example 89

### 2-({3-[3-benzyl-8-(trifluoromethyl)quinolin-4-yl]benzyl}oxy)-6-fluorobenzoic acid

MS (ES) m/z 531.7; Prepared in a manner similar to Example 5.

### Example 90

### 3-({3-[3-benzyl-8-(trifluoromethyl)quinolin-4-yl]phenoxy}methyl)benzoic acid

MS (ES) m/z 514.1; Prepared in a manner similar to Example 5.

### Example 91

### 2-({3-[3-benzyl-8-(trifluoromethyl)quinolin-4-yl]benzyl}amino)-4-chlorobenzoic acid

MS (ES) m/z 547.3; Prepared in a manner similar to Example 12.

### Example 92

### 3-({3-[3-benzyl-8-(trifluoromethyl)quinolin-4-yl]benzyl}oxy)-4-fluorobenzoic acid

MS (ES) m/z 531.8; Prepared in a manner similar to Example 5.

### Example 93

### 2-({3-[3-benzyl-8-(trifluoromethyl)quinolin-4-yl]benzyl}amino)-6-methylbenzoic acid

MS (ES) m/z 526.8; Prepared in a manner similar to Example 12.

### Example 94

### 3-({3-[3-benzyl-8-(trifluoromethyl)quinolin-4-yl]benzyl}amino)-4-methylbenzoic acid

MS (ES) m/z 527.3; Prepared in a manner similar to Example 12.

### Example 95

### 1-({3-[3-benzyl-8-(trifluoromethyl)quinolin-4-yl]benzyl}oxy)-2-naphthoic acid

MS (ES) m/z 563.8; Prepared in a manner similar to Example 5.

### Example 96

### N-[3-({3-[3-benzyl-8-(trifluoromethyl)quinolin-4-yl]phenoxy}methyl)benzoyl]leucine

MS (ESI) m/z 627; Prepared in a manner similar to Example 11.

### Example 97

### 2-({3-[3-benzyl-8-(trifluoromethyl)quinolin-4-yl]benzyl}amino)benzoic acid

MS (ES) m/z 513.2; Prepared in a manner similar to Example 12.

### Example 98

### 3-({3-[3-benzyl-8-(trifluoromethyl)quinolin-4-yl]benzyl}oxy)-4-methoxybenzoic acid

MS (ES) m/z 541.8; Prepared in a manner similar to Example 5.

### Example 99

### 5-({3-[3-benzyl-8-(trifluoromethyl)quinolin-4-yl]benzyl}amino)-1H-indole-3-carboxylic acid

MS (ES) m/z 551.9; Prepared in a manner similar to Example 12.

### Example 100

### 4-({3-[3-benzyl-8-(trifluoromethyl)quinolin-4-yl]phenoxy}methyl)benzoic acid

MS (ES) m/z 514.1; Prepared in a manner similar to Example 5.

### Example 101

### 4-({3-[3-benzyl-8-(trifluoromethyl)quinolin-4-yl]benzyl}amino)benzoic acid

MS (ES) m/z 513.3; Prepared in a manner similar to Example 12.

### Example 102

### 2-({3-[3-benzyl-8-(trifluoromethyl)quinolin-4-yl]benzyl}oxy)-5-methylbenzoic acid

MS (ES) m/z 525.8; Prepared in a manner similar to Example 5.

### Example 103

### 4-({3-[3-benzyl-8-(trifluoromethyl)quinolin-4-yl]benzyl}amino)-2,3-dimethylbenzoic acid

MS (ES) m/z 541.0; Prepared in a manner similar to Example 12.

### Example 104

### 2-({3-[3-benzyl-8-(trifluoromethyl)quinolin-4-yl]benzyl}oxy)-5-fluorobenzoic acid

MS (ES) m/z 531.8; Prepared in a manner similar to Example 5.

### Example 105

### 7-({3-[3-benzyl-8-(trifluoromethyl)quinolin-4-yl]benzyl}amino)-1-methyl-1H-indole-2-carboxylic acid

MS (ES) m/z 565.7; Prepared in a manner similar to Example 12.

### Example 106

### 4-fluoro-2-({3-[3-methyl-8-(trifluoromethyl)quinolin-4-yl]benzyl}amino)benzoic acid

MS (ES) m/z 452.8; Prepared in a manner similar to Example 12.

### Example 107

### [7-({3-[8-(trifluoromethyl)quinolin-4-yl]benzyl}oxy)-1-naphthyl]acetic acid

MS (ES) m/z 488.1; Prepared in a manner similar to Example 5.

### Example 108

### 4-({3-[3-benzyl-8-(trifluoromethyl)quinolin-4-yl]benzyl}amino)-5-bromo-2,3-dimethylbenzoic acid

MS (ES) m/z 618.9; Prepared in a manner similar to Example 12.

### Example 109

### 3-({3-[3-benzyl-8-(trifluoromethyl)quinolin-4-yl]benzyl}oxy)-2-naphthoic acid

MS (ES) m/z 563.8; Prepared in a manner similar to Example 5.

### Example 110

### 4-({3-[3-benzyl-8-(trifluoromethyl)quinolin-4-yl]benzyl}amino)-3-(trifluoromethoxy)benzoic acid

MS (ES) m/z 596.9; Prepared in a manner similar to Example 12.

### Example 111

### 2-({3-[3-benzyl-8-(trifluoromethyl)quinolin-4-yl]benzyl}amino)-6-chlorobenzoic acid

MS (ES) m/z 546.8; Prepared in a manner similar to Example 12.

### Example 112

### 2-({3-[3-benzyl-8-(trifluoromethyl)quinolin-4-yl]benzyl}amino)-5-fluorobenzoic acid

MS (ES) m/z 528.7; Prepared in a manner similar to Example 12.

### Example 113

### 4-({3-[3-benzyl-8-(trifluoromethyl)quinolin-4-yl]phenoxy}methyl)-3-chlorobenzoic acid

MS (ES) m/z 547.8; Prepared in a manner similar to Example 5.

### Example 114

### [8-({3-[3-benzyl-8-(trifluoromethyl)quinolin-4-yl]benzyl}oxy)-2-naphthyl]acetic acid

MS (ES) m/z 578.0; Prepared in a manner similar to Example 5.

### Example 115

### 2-fluoro-6-({3-[3-methyl-8-(trifluoromethyl)quinolin-4-yl]benzyl}amino)benzoic acid

MS (ES) m/z 454.8; Prepared in a manner similar to Example 12.

### Example 116

### {5-[({5-[8-(trifluoromethyl)quinolin-4-yl]pyridin-3-yl}methyl)amino]-1-naphthyl}acetic acid

MS (ES) m/z 488.0; Prepared in a manner similar to Example 1.

### Example 117

### {2,5-dimethyl-4-[({5-[3-methyl-8-(trifluoromethyl)quinolin-4-yl]pyridin-3-yl}methyl)amino]phenyl}acetic acid

MS (ES) m/z 480.1; Prepared in a manner similar to Example 2.

### Example 118

### [5-({5-[8-(trifluoromethyl)quinolin-4-yl]pyridin-3-yl}methoxy)-1-naphthyl]acetic acid

MS (ES) m/z 489.0; Prepared in a manner similar to Example 5.

### Example 119

### {5-[({5-[3-methyl-8-(trifluoromethyl)quinolin-4-yl]pyridin-3-yl}methyl)amino]-1-naphthyl}acetic acid

MS (ES) m/z 502.1; Prepared in a manner similar to Example 2.

### Example 120

### {4-[({5-[8-(trifluoromethyl)quinolin-4-yl]pyridin-3-yl}methyl)amino]phenyl}acetic acid

MS (ES) m/z 438.1; Prepared in a manner similar to Example 2.

### Example 121

### 5-{3-[3-Benzyl-8-(trifluoromethyl)quinolin-4-yl]phenoxy}pyrazine-2-carboxylic acid

A mixture of 3-[3-benzyl-8-(trifluoromethyl)quinolin-4-yl]phenol (US 2005131014) (0.50 g, 1.32 mmol), methyl 5-chloropyrazine-2-carboxylate (0.50 g, 2.9 mmol), cesium carbonate (3.0 g, 9.2 mmol) in DMF (25 ml) was heated to 110° C. 1.5 hrs. The reaction was quenched with water and extracted with ethyl acetate. The organic layers were concentrated and the residue was purified on silica gel chromatography to afforded 5-{3-[3-benzyl-8-(trifluoromethyl)quinolin-4-yl]phenoxy}pyrazine-2-carboxylic acid methyl ester (0.40 g, 59% yield) as a white solid. To this same ester was dissolved up in Methanol/THF/H₂O (2:1:1, 15 ml) was added lithium hydroxide (0.1 g) and heated at 40° C. for 2 hrs. The reaction was cooled to room temperature upon which was quenched with acetic acid (pH<4) and concentrated in vacuo to afforded the crude carboxylic acid. This same crude acid was subjected to reversed phased chromatography (AcCN:H₂O) to afford the title compound as a white powder (21 mg, 72% yield); MS (ES) m/z 501.9.

### Example 122

### 3-[3-(3-Benzyl-8-chloroquinolin-4-yl)phenoxy]-5-bromobenzoic acid

The title compound was prepared following the procedure of Example 121 as a yellow solid; HRMS [M+H]+: 544.0298.

### Example 123

### 3-[3-(3-Benzyl-8-chloroquinolin-4-yl)phenoxy]-5-fluorobenzoic acid

The title compound was prepared following the procedure of Example 121 as a yellow foamy solid; HRMS [M+H]+: 484.1111.

### Example 124

### 4-{3-[3-Phenyl-8-(trifluoromethyl)quinolin-4-yl]phenoxy}benzoic acid

Step 1: 3-[3-Phenyl-8-(trifluoromethyl)quinolin-4-yl]phenol (0.763 g, 2.21 mmol), 3-methoxycarbonylphenylboronic acid (0.874 g, 4.85 mmol), copper acetate (441 g, 2.43 mmol), and powdered 4 Å molecular sieves was stirred at R.T. in CH₂Cl₂ (26 mL) for 10 minutes. Triethylamine (1.53 mL, 11.1 mmol) was added and the reaction was stirred at R.T. for 24 hours. The reaction was filtered through an Autovial filter directly onto a Horizon silica gel samplet. Chromatography on silica gel eluting with an ethyl acetate/hexane gradient of 0/100 to 5/95 affords methyl 4-{3-[3-phenyl-8-(trifluoromethyl)quinolin-4-yl]phenoxy}benzoate as a colorless oil (697 mg); MS (ES) m/z 499.9;

Step 2: Methyl 4-{3-[3-phenyl-8-(trifluoromethyl)quinolin-4-yl]phenoxy}benzoate (0.061 g, 0.122 mmol) was dissolved in (1:1) MeOH:THF (5 mL). 2N NaOH (2 mL, 4 mmol) was added and the solution was refluxed for 1 hour. The reaction was cooled, poured into 2N HCl, and extracted with ethyl acetate (3×5 mL). The extracts were washed with H₂O, brine, and dried with MgSO₄ and concentrated in vacuo to afford the title compound as a white solid (50 mg); MS (ES) m/z 483.9.

### Example 125

### 3-[3-(3-Benzyl-8-chloroquinolin-4-yl)phenoxy]benzoic acid

The title compound was prepared following the procedure of Example 121 as a light yellow solid; MS (ES) m/z 463.8.

### Example 126

### 3-{3-[3-Benzyl-8-(trifluoromethyl)quinolin-4-yl]phenoxy}benzoic acid

The title compound was prepared following the procedure of Example 121 as a white flacky foam; MS (ES) m/z 500.1.

### Example 127

### 4-({4-[3-Benzyl-8-(trifluoromethyl)quinolin-4-yl]-1H-indol-1-yl}methyl)benzoic acid

In a one necked flask open to the atmosphere at room temperature was placed 3-benzyl-4-bromo-8-(trifluoromethyl)quinoline (748 mg, 2.050 mmol), 1H-indol-4-ylboronic acid (300 mg, 1.863 mmol), sodium carbonate (593 mg, 5.590 mmol), tetrakis(triphenylphosphine) palladium (430 mg, 0.373 mmol), toluene (25 ml), water (12 ml), and ethyl alcohol (6 ml). The resulting yellow bi-phased mixture was heated at reflux for 2 hrs. Cooling to room temperature upon which this mixture was partitioned between water and EtOAc (25 mL each). All organics combined, dried over MgSO₄, filtration and concentration in vacuo to a brown powder. This same brown powder was subjected to reversed phased chromatography (AcCN:H₂O) to afforded a red powder 3-benzyl-4-(1H-indol-4-yl)-8-(trifluoromethyl)quinoline (167 mg, 37% yield).

In a one necked flask under a nitrogen atmosphere at room temperature was placed 3-benzyl-4-(1H-indol-4-yl)-8-(trifluoromethyl)quinoline (20 mg, 0.050 mmol) in DMF (3 ml) and sodium hydride (1.4 mg, 0.06 mmol). The resulting red mixture was stirred 30 min upon which was added methyl 4-(bromomethyl)benzoate (14.8 mg, 0.065 mmol). The resulting brown mixture was stirred at room temperature for 1 hr. Quenching with water a few drops followed by 3 ml and partitioned with EtOAc (5 ml). All organics combined, dried over MgSO₄, filtration and concentration in vacuo to a brown powder. This same brown powder was subjected to reversed phased chromatography (AcCN:H₂O) to afforded a brown powder methyl 4-((4-(3-benzyl-8-(trifluoromethyl) quinolin-4-yl)-1H-indol-1-yl)methyl)benzoate (8 mg, 29% yield). To this same ester (8 mg, 0.015 mmol) was dissolved up in THF:H₂O/4:1 (3 ml) was added lithium hydroxide (1.7 mg, 0.045 mmol) and heated at 50° C. for 3 hrs. The reaction was cooled to room temperature upon which was quenched with 1N HClaq (pH <2) and concentrated in vacuo to afforded the crude carboxylic acid as a brown powder. This same crude acid was subjected to reversed phased chromatography (AcCN:H₂O) to afford a brown powder (4 mg, 55% yield) of the title compound; MS (ES) m/z 534.8.

### Example 128

### 4-({3-[3-Benzyl-8-(trifluoromethyl)quinolin-4-yl]phenyl}ethynyl)-3-methylbenzoic acid

Step 1: A solution of 3-benzyl-4-(3-bromo-phenyl)-8-trifluoromethyl-quinoline (1.0 g, 23 mmol) and trimethyl-tributylstannanylethynyl-silane (1.3 g, 34 mmol) in toluene (25 mL) is treated with Pd(PPh₃)₄ (270 mg) and heated at 120° C. for 3 h. The reaction is then cooled and concentrated in vacuo. The residue is chromatographed with 10:90 ethyl acetate:hexane to afford 3-benzyl-8-trifluoromethyl-4-(3-trimethylsilanylethynyl-phenyl)-quinoline as an oil; MS (ESI) m/z 460.0;

A solution of the above oil, 3-benzyl-8-trifluoromethyl-4-(3-trimethylsilanylethynyl-phenyl)-quinoline (730 mg, 1.6 mmol), methyl 4-iodo-3-methylbenzoate, (100 mg, 0.4 mmol), PdCl₂(PPh₃)₂ (8.0 mg), and piperidine (63 mg, 0.7 mmol) in toluene (30 mL) is stirred at ambient temperature for 3 h. The reaction mixture is stripped to dryness and the residue taken up in ethyl acetate and washed with 30 mL (1N HCl). The organic layer is dried and concentrated in vacuo to provide after chromatography 8 mg of methyl 4-((3-(3-benzyl-8-(trifluoromethyl)quinolin-4-yl)phenyl)ethynyl)-3-methylbenzoate; MS (ES) m/z 536.2;

Step 3: The above methyl ester (25 mg, 0.05 mmol) in MeOH:Ether (4 mL, 3:1) was treated with 0.5 mL 15% NaOH solution and heated to 65° C. for 3 h. The reaction mixture was cooled to RT and acidified with 1 N HCl and extracted with ethylacetate.

The organic layer is dried and concentrated in vacuo to provide after chromatography the title compound as a white solid (19 mg 73%). MS (ESI) m/z 522.

### Example 129

### 3-({3-[3-Benzyl-8-(trifluoromethyl)quinolin-4-yl]phenyl}ethynyl)-4-methylbenzoic acid

The title compound was prepared following the procedure of Example 128 as a white solid; MS (ESI) m/z 522.

### Example 130

### 3′-[3-Benzyl-8-(trifluoromethyl)quinolin-4-yl]biphenyl-3-carboxylic acid

Step 1: 3-Benzyl-4-(3-bromophenyl)-8-(trifluoromethyl)quinoline (300 mg, 0.68 mmol) was taken into toluene/EtOH (3 mL/0.5 mL). Then 3-(methoxycarbonyl)phenylboronic acid (0.183 mg, 1.0 mmol) was added followed by 2 M Na₂CO₃ (1.7 mL, 3.4 mmol) and finally Pd(PPh₃)₄ (39 mg, 0.034 mmol). The reaction was heated at 90° C. for 4 hours. The solvent was removed and the resulting material was purified via column chromatography using 5% ethyl acetate in hexane to elute out (0.235 g, 70%) of methyl 3′-[3-benzyl-8-(trifluoromethyl)quinolin-4-yl]biphenyl-3-carboxylate; MS (ES) m/z 497.9.

Step 2: The above methyl ester (233 mg, 0.5 mmol) in MeOH (40 mL) was treated with 1.0 mL 2N NaOH solution and heated to 90° C. for 3 h. The reaction mixture was cooled to RT and acidified with 1 N HCl and extracted with ethylacetate. The organic layer is dried and concentrated in vacuo to provide after chromatography the title compound as a white solid (180 mg 79%); MS (ES) m/z 484.1.

### Example 131

### 3-({3-[3-Benzyl-8-(trifluoromethyl)quinolin-4-yl]phenyl}ethynyl)-4-methoxybenzoic acid

The title compound was prepared following the procedure of Example 128 as a white solid; MS (ESI) m/z 538.

### Example 132

### Biological Testing

Representative compounds of this invention were evaluated in conventional pharmacological test procedures which measured their affinity to bind to LXR and to upregulate the gene ABCA1, which causes cholesterol efflux from atherogenic cells, such as macrophages.

LXR activation can be critical for maintaining cholesterol homeostasis, but its coincident regulation of fatty acid metabolism may lead to increased serum and hepatic triglyceride levels. Selective LXR modulators that activate cholesterol efflux with minimal impact on SREBP-1c expression and triglyceride synthesis in liver would be expected to reduce atherosclerotic risk with an improved therapeutic index and minimize the potential for deleterious effects on metabolic balance.

Accordingly, LXR ligands were identified initially in cell-free LXR beta and LXR alpha competition binding assays. LXR ligands were further characterized by gene expression profiling for tissue selective gene regulation. Selective LXR modulators demonstrate agonist activity for ABCA1 transactivation.

The test procedures performed, and results obtained are briefly described below.

### Ligand-Binding Test Procedure for Human LXRβ.

Ligand-binding to the human LXRβ was demonstrated for representative compounds of this invention by the following procedure.

### Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

### Tracer: ³H T0901317

Receptor source: E. coli extract from cells expressing biotinylated hLXRβ. Extract was made in a similar buffer as above, but with 50 mM TRIS.

### Day 1

Washed streptavidin and coated flash plates with wash buffer.

Diluted receptor extract to give Bmax ˜4000 cpm and add to the wells.

Wrapped the plates in aluminum foil and stored them at +4° C. over night.

### Day 2

Made a dilution series in DMSO of the test ligands.

Made a 5 nM solution of the radioactive tracer in buffer.

Mixed 250 μl diluted tracer with 5 μl of the test ligand from each concentration of the dilution series.

Washed the receptor-coated flash plates.

Added 200 μl per well of the ligand/radiolabel mixture to the receptor-coated flash plates.

Wrapped the plates in aluminum foil and incubate at +4° C. over night.

### Day 3

Aspirated wells, and wash the flashed plates. Sealed the plate.

Measured the remaining radioactivity in the plate.

### Ligand-Binding Test Procedure for Human LXRα.

Ligand-binding to the human LXRα was demonstrated for representative compounds of this invention by the following procedure.

Materials and Methods:

Buffer: 100 mM KCl, 100 mM TRIS (pH 7.4 at +4° C.), 8.6% glycerol, 0.1 mM PMSF*, 2 mM MTG*, 0.2% CHAPS (* not used in wash buffer)

Tracer: ³H T0901317

Receptor source: E. coli extract from cells expressing biotinylated hLXRα. Extract was made in a similar buffer as above, but with 50 mM TRIS.

Day 1

Washed streptavidin and coated flash plates with wash buffer.

Diluted receptor extract to give Bmax ˜4000 cpm and add to the wells.

Wrapped the plates in aluminum foil and stored them at +4° C. over night.

Day 2

Made a dilution series in DMSO of the test ligands.

Made a 5 nM solution of the radioactive tracer in buffer.

Mixed 250 μl diluted tracer with 5 μl of the test ligand from each concentration of the dilution series.

Washed the receptor-coated flash plates.

Added 200 μl per well of the ligand/radiolabel mixture to the receptor-coated flash plates.

Wrapped the plates in aluminum foil and incubate at +4° C. over night.

Day 3

Aspirated wells, and wash the flashed plates. Sealed the plate.

Measured the remaining radioactivity in the plate.

Results:

TABLE 1

| | LXRβ binding | LXRα binding | Binding ratio |
|---|---|---|---|
| Example | IC50 (uM) | IC50 (uM) | a/b |
| 1 | 0.015 | 0.745 | 48 |
| 2 | 0.005 | 0.185 | 36 |
| 3 | 0.03 | 0.964 | 32 |
| 4 | 0.288 | 9.031 | 31 |
| 5 | 0.026 | 0.779 | 30 |
| 6 | 0.182 | 4.511 | 25 |
| 7 | 0.009 | 0.223 | 24 |
| 8 | 0.005 | 0.123 | 23 |
| 9 | 0.013 | 0.294 | 22 |
| 10 | 0.028 | 0.615 | 22 |
| 13 | 0.01 | 0.211 | 22 |
| 14 | 0.148 | 3.206 | 22 |
| 15 | 0.117 | 2.157 | 18 |
| 16 | 0.295 | 5.186 | 18 |
| 17 | 0.003 | 0.048 | 18 |
| 18 | 0.012 | 0.201 | 16 |
| 19 | 0.008 | 0.133 | 16 |
| 20 | 0.004 | 0.058 | 14 |
| 21 | 0.011 | 0.161 | 14 |
| 22 | 1.366 | 17.55 | 13 |
| 23 | 0.062 | 0.667 | 11 |
| 12 | 0.012 | 0.132 | 11 |
| 24 | 0.002 | 0.017 | 11 |
| 25 | 0.104 | 1.066 | 10 |
| 26 | 0.032 | 0.325 | 10 |
| 27 | 0.034 | 0.333 | 10 |
| 28 | 0.007 | 0.066 | 9 |
| 29 | 0.023 | 0.211 | 9 |
| 30 | 0.009 | 0.072 | 8 |
| 31 | 0.21 | 1.739 | 8 |
| 32 | 0.427 | 3.332 | 8 |
| 33 | 0.166 | 1.291 | 8 |
| 34 | 0.004 | 0.032 | 8 |
| 35 | 0.131 | 0.961 | 7 |
| 36 | 0.191 | 1.357 | 7 |
| 128 | 0.019 | 0.137 | 7 |
| 37 | 0.019 | 0.131 | 7 |
| 38 | 0.008 | 0.051 | 7 |
| 39 | 0.101 | 0.683 | 7 |
| 40 | 0.084 | 0.559 | 7 |
| 41 | 0.05 | 0.331 | 7 |
| 42 | 0.007 | 0.047 | 6 |
| 43 | 0.002 | 0.015 | 6 |
| 44 | 0.165 | 1.048 | 6 |
| 45 | 0.358 | 2.255 | 6 |
| 46 | 1.305 | 8.002 | 6 |
| 47 | 0.002 | 0.013 | 6 |
| 48 | 0.538 | 3.273 | 6 |
| 49 | 0.066 | 0.393 | 6 |
| 50 | 0.004 | 0.02 | 6 |
| 51 | 0.209 | 1.17 | 6 |
| 52 | 0.249 | 1.375 | 6 |
| 53 | 0.095 | 0.51 | 5 |
| 54 | 0.062 | 0.321 | 5 |
| 55 | 0.365 | 1.807 | 5 |
| 56 | 0.032 | 0.157 | 5 |
| 57 | 0.055 | 0.267 | 5 |
| 58 | 0.025 | 0.121 | 5 |
| 59 | 0.098 | 0.467 | 5 |
| 60 | 0.038 | 0.178 | 5 |
| 61 | 0.145 | 0.681 | 5 |
| 62 | 0.027 | 0.125 | 5 |
| 63 | 0.332 | 1.529 | 5 |
| 64 | 0.813 | 3.517 | 4 |
| 65 | 2.054 | 8.831 | 4 |
| 66 | 0.454 | 1.949 | 4 |
| 67 | 0.047 | 0.203 | 4 |
| 68 | 0.535 | 2.286 | 4 |
| 69 | 0.071 | 0.276 | 4 |
| 70 | 0.002 | 0.007 | 4 |
| 71 | 0.558 | 2.064 | 4 |
| 72 | 0.309 | 1.103 | 4 |
| 73 | 0.183 | 0.613 | 3 |
| 74 | 0.003 | 0.01 | 3 |
| 75 | 0.025 | 0.08 | 3 |
| 76 | 0.318 | 0.983 | 3 |
| 77 | 0.408 | 1.253 | 3 |
| 78 | 0.666 | 1.988 | 3 |
| 11 | 0.14 | 0.418 | 3 |
| 79 | 0.629 | 1.821 | 3 |
| 80 | 0.129 | 0.374 | 3 |
| 81 | 0.236 | 0.654 | 3 |
| 82 | 0.523 | 1.449 | 3 |
| 83 | 0.292 | 0.802 | 3 |
| 84 | 0.319 | 0.864 | 3 |
| 85 | 0.359 | 0.966 | 3 |
| 86 | 0.374 | 0.997 | 3 |
| 87 | 0.028 | 0.072 | 3 |
| 88 | 12.69 | 31.6 | 2 |
| 89 | 1.603 | 3.98 | 2 |
| 90 | 0.358 | 0.872 | 2 |
| 91 | 0.477 | 1.079 | 2 |
| 92 | 0.57 | 1.277 | 2 |
| 93 | 0.906 | 1.999 | 2 |
| 94 | 1.371 | 3.015 | 2 |
| 95 | 1.028 | 2.225 | 2 |
| 96 | 0.546 | 1.165 | 2 |
| 97 | 0.237 | 0.493 | 2 |
| 98 | 2.503 | 5.195 | 2 |
| 99 | 0.62 | 1.244 | 2 |
| 122 | 0.772 | 1.512 | 2 |
| 100 | 0.053 | 0.103 | 2 |
| 101 | 0.183 | 0.354 | 2 |
| 126 | 0.135 | 0.241 | 2 |
| 131 | 1.475 | 2.634 | 2 |
| 102 | 2.821 | 4.756 | 2 |
| 103 | 3.297 | 5.038 | 2 |
| 123 | 0.481 | 0.723 | 2 |
| 104 | 2.146 | 3.201 | 1 |
| 105 | 0.129 | 0.185 | 1 |
| 106 | 5.295 | 7.278 | 1 |
| 124 | 0.701 | 0.951 | 1 |
| 107 | 5.543 | 7.383 | 1 |
| 108 | 2.729 | 3.618 | 1 |
| 109 | 2.733 | 3.59 | 1 |
| 110 | 1.578 | 2.045 | 1 |
| 111 | 0.709 | 0.874 | 1 |
| 125 | 0.228 | 0.267 | 1 |
| 112 | 0.464 | 0.517 | 1 |
| 113 | 1.564 | 1.715 | 1 |
| 129 | 0.605 | 0.656 | 1 |
| 114 | 1.19 | 1.29 | 1 |
| 115 | 10.34 | 10.67 | 1 |
| 127 | 2.462 | 2.139 | 1 |
| 121 | 2.296 | 1.707 | 1 |
| 116 | 15.71 | >10 | |
| 130 | 2.377 | 1.322 | |
| 117 | 2.521 | >1 | |
| 118 | 3.739 | >1 | |
| 119 | 7.217 | >1 | |
| 120 | >1 | >1 | |

### Quantitative Analysis of ABCA1 Gene Regulation in J774 Cells.

The compounds of formula (I) effect on the regulation of the ABCA1 gene was evaluated using the following procedure.

### Materials and Methods

Cell culture: The J774.A1 cell line (ATCC # TIB-67) was obtained from American Type Culture Collection (Manassas, Va.) and cultured in RPMI 1640 medium (Gibco, Carlsbad, Calif.) containing 10% FBS, 4 mM glutamax, and antibiotics (Gibco, BRL). Cells were plated in 96-well format at a density of 6.0×10⁴ per well. J774.A1 cells were treated with test compounds or ligands dissolved in DMSO (Sigma, D-8779) in culture medium. Final concentrations of DMSO did not exceed 0.3% of the media volume. Dose response effects were measured in duplicate, in the range of 0.001 to 30 micromolar concentrations and treated cells were incubated for an additional 18 hrs prior to RNA isolation. Unstimulated cells treated with vehicle were included as negative controls on each plate. An LXR agonist reference, N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838), was dosed at 1.0 μM and served as a positive control.
RNA isolation and quantitation: Total cellular RNA was isolated from treated cells cultured in 96-well plates using PrepStation 6100 (Applied Biosystems, Foster City, Calif.), according to the manufacturer's recommendations. RNA was resuspended in ribonuclease-free water and stored at −70° C. prior to analysis. RNA concentrations were quantitated with RiboGreen test procedure, #R-11490 (Molecular Probes, Eugene, Oreg.).
Gene expression analysis: Gene-specific mRNA quantitation was performed by real-time PCR with the Perkin Elmer Corp. chemistry on an ABI Prism 7900 Sequence detection system (Applied Biosystems, Foster City, Calif.) according to the manufacturer's instructions. Samples (50-100 ng) of total RNA were assayed in duplicate or triplicate in 50 ul reactions using one-step RT-PCR and the standard curve method to estimate specific mRNA concentrations. Sequences of gene-specific primer and probe sets were designed with Primer Express Software (Applied Biosystems, Foster City, Calif.). The murine ABCA1 primer and probe sequences are: forward, GAAGCCAGTTGTGCAAAACTAAATT, reverse, GCAACACTGTGGTGGCTTCA, and probe, 6FAM-CACATCTCATCTCCCGACCCAGCA-TAMRA. The RT and PCR reactions were performed according to PE Applied Biosystem's protocol for Taqman Gold RT-PCR or Qiagen's protocol for Quantitect probe RT-PCR. Relative levels of ABCA1 mRNA are normalized with murine β-actin mRNA, GAPDH mRNA, or 18S rRNA. The murine β-actin primer and probe sequences are: forward, AGCCATGTACGTAGCCATCCA, reverse, TCTCCGGAGTCCATCACAATG, and probe, 6FAM-TGTCCCTGTATGCCTCTGGTCGTACCAC-TAMRA. The GAPDH mRNA or 18S rRNA probe/primer sets were purchased commercially (Applied Biosystems, Foster City, Calif.).

### Statistics:

Mean, standard deviation and statistical significance of duplicate evaluations of RNA samples were assessed using ANOVA, one-way analysis of variance using SAS analysis.

### Reagents:

### Rodent GAPDH Probe and Primers—Taqman GAPDH Control Reagents 4308313

### 18S Ribosomal RNA—Taqman 18S Control Reagents 4308329

### 10 Pack Taqman PCR Core Reagent Kit 402930

Qiagen Quantitect probe RT-PCR 204445.

### Quantitative Analysis of ABCA1 Gene Regulation in THP-1 Cells.

The compounds of formula (I) effect on the regulation of the ABCA1 gene was evaluated using the following procedure.

### Materials and Methods

Cell culture: The THP-1 monocytic cell line (ATCC # TIB-202) was obtained from American Type Culture Collection (Manassas, Va.) and cultured in RPMI 1640 medium (Gibco, Carlsbad, Calif.) containing 10% FBS, 2 mM L-glutamine, and 55 uM beta-Mercaptoethanol (BME). Cells were plated in 96-well format at a density of 7.5×10⁴ in complete medium containing 50-100 ng/ml phorbal 12,13-dibutyrate (Sigma, St. Louis, Mo.) for three days to induce differentiation into adherent macrophages. Differentiated THP-1 cells were treated with test compounds or ligands dissolved in DMSO (Sigma, D-8779) in culture medium lacking phorbal ester. Final concentrations of DMSO did not exceed 0.3% of the media volume. Dose response effects were measured in duplicate, in the range of 0.001 to 30 micromolar concentrations and treated cells were incubated for an additional 18 hrs prior to RNA isolation. Unstimulated cells treated with vehicle were included as negative controls on each plate. An LXR agonist reference, N-(2,2,2-trifluoro-ethyl)-N-[4-(2,2,2-trifluoro-1-hydroxy-1-trifluoromethyl-ethyl)-phenyl]-benzenesulfonamide (Schultz, Joshua R., Genes & Development (2000), 14(22), 2831-2838), was dosed at 1.0 uM and served as a positive control. In antagonist mode, the compound under study is analyzed in the presence of 150 nM GW3965, trifluoromethyl-benzyl)-(2,2-diphenyl-ethyl)-amino]-propoxy]-phenyl)-acetic acid (Collins, J. L., J. Med. Chem. (2000), 45:1963-1966.). Results of antagonist analysis are expressed as % antagonism and IC50 (in μM).
RNA isolation and quantitation: Total cellular RNA was isolated from treated cells cultured in 96-well plates using PrepStation 6100 (Applied Biosystems, Foster City, Calif.), according to the manufacturer's recommendations. RNA was resuspended in ribonuclease-free water and stored at −70° C. prior to analysis. RNA concentrations were quantitated with RiboGreen test procedure, #R-11490 (Molecular Probes, Eugene, Oreg.).
Gene expression analysis: Gene-specific mRNA quantitation was performed by real-time PCR with the Perkin Elmer Corp. chemistry on an ABI Prism 7700 Sequence detection system (Applied Biosystems, Foster City, Calif.) according to the manufacturer's instructions. Samples (50-100 ng) of total RNA were assayed in duplicate or triplicate in 50 ul reactions using one-step RT-PCR and the standard curve method to estimate specific mRNA concentrations. Sequences of gene-specific primer and probe sets were designed with Primer Express Software (Applied Biosystems, Foster City, Calif.). The human ABCA1 primer and probe sequences are: forward, CAACATGAATGCCATTTTCCAA, reverse, ATAATCCCCTGAACCCAAGGA, and probe, 6FAM-TAAAGCCATGCCCTCTGCAGGAACA-TAMRA. RT and PCR reactions were performed according to PE Applied Biosystem's protocol for Taqman Gold RT-PCR or Qiagen's protocol for Quantitect probe RT-PCR. Relative levels of ABCA1 mRNA are normalized using GAPDH mRNA or 18S rRNA probe/primer sets purchased commercially (Applied Biosystems, Foster City, Calif.).

### Statistics:

Mean, standard deviation and statistical significance of duplicate evaluations of RNA samples were assessed using ANOVA, one-way analysis of variance using SAS analysis.

### Reagents:

### GAPDH Probe and Primers—Taqman GAPDH Control Reagents 402869 or 4310884E

### 18S Ribosomal RNA—Taqman 18S Control Reagents 4308329

### 10 Pack Taqman PCR Core Reagent Kit 402930

Qiagen Quantitect probe RT-PCR 204443.

### Results:

TABLE 2

| | J774 cells | | THP cells | |
|---|---|---|---|---|
| | ABCA1 | | ABCA1 | |
| Example | EC50 (uM) | Ag. (%) | EC50 (uM) | Ag. (%) |
| 1 | 0.669 | 75.1887 | 3.76 | 91.15 |
| 2 | 0.056 | 95.85 | | |
| 3 | 0.727 | 93.45 | 4.76 | 69.1 |
| 5 | 2.784 | 103.95 | 3.39 | 69.4 |
| 6 | 1.561 | 82 | | |
| 7 | 0.052 | 86.3 | | |
| 8 | 0.039 | 90.7 | | |
| 9 | 0.145 | 130.05 | 1.135 | 122.6 |
| 10 | 0.1155 | 106.95 | | |
| 13 | 0.475 | 97.45 | 2.63 | 107.8 |
| 14 | 0.63 | 94.6 | | |
| 15 | 2.6353 | 100.133 | | |
| 17 | 0.0037 | 95.8 | | |
| 18 | 0.064 | 65.25 | | |
| 19 | 0.1245 | 130.7 | | |
| 20 | 0.121 | 131.9 | | |
| 21 | 0.0123 | 101.033 | | |
| 23 | 1.6937 | 122.6 | | |
| 12 | 0.161 | 113.65 | | |
| 24 | 0.023 | 72 | | |
| 25 | 0.9355 | 87.25 | | |
| 26 | 0.141 | 118 | | |
| 27 | 0.542 | 134 | | |
| 28 | 0.0315 | 95.45 | | |
| 29 | 0.207 | 92.9 | | |
| 30 | 0.538 | 91.6 | | |
| 33 | 1.247 | 86.6 | | |
| 34 | 0.0025 | 118 | | |
| 128 | 0.433 | 243 | | |
| 41 | 1.8365 | 134.45 | | |
| 58 | 0.822 | 95.9 | | |
| 67 | 0.1325 | 102 | | |
| 70 | 0.0005 | 94.6 | | |
| 87 | 0.5676 | 131.2 | | |
| 100 | 0.6481 | 90.8 | | |

Based on the results obtained in the standard pharmacological test procedures, the compounds of this invention can be useful in treating or inhibiting LXR mediated diseases. In particular, the compounds of this invention can be useful in the treatment and inhibition of atherosclerosis and atherosclerotic lesions, lowering LDL cholesterol levels, increasing HDL cholesterol levels, increasing reverse cholesterol transport, inhibiting cholesterol absorption, treatment or inhibition of Alzheimer's disease, type I diabetes, type II diabetes, multiple sclerosis, rheumatoid arthritis, acute coronary syndrome, restenosis, inflammatory bowel disease (IBD), Crohn's disease, endometriosis, celiac, and thyroiditis.

A number of embodiments of the invention have been described. Nevertheless, it will be understood that various modifications may be made without departing from the spirit and scope of the invention. Accordingly, other embodiments are in the claims.

What is claimed is:

1. A compound having formula (I): 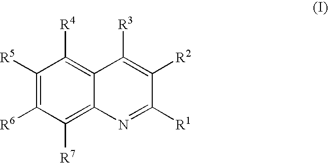
wherein:
R¹ is hydrogen or C₁-C₆ alkyl;
R² is:
(i) hydrogen; or
(ii) C₁-C₁₂ alkyl or C₁-C₁₂ haloalkyl, each of which is optionally substituted with from 1-5 R^{a}; or
(iii) C₇-C₂₀ aralkyl or heteroaralkyl including 6-20 atoms, each of which is optionally substituted with from 1-10 R^{b}; or
(iv) C₂-C₁₂ alkenyl or C₂-C₁₂ alkynyl, each of which is optionally substituted with from 1-10 R^{c};
(v) C₃-C₁₀ cycloalkyl or heterocyclyl including 3-10 atoms, each of which is optionally substituted with from 1-5 R^{b}; or
(vi) C₆-C₁₈ aryl or heteroaryl including 5-16 atoms, each of which is optionally substituted with from 1-10 R^{d};
R³ is C₆-C₁₈ aryl, heteroaryl including 5-16 atoms, C₈-C₂₀ arylcycloalkyl, heteroarylcycloalkyl including 8-20 atoms, C₈-C₂₀ arylcycloalkenyl, heteroarylcycloalkenyl including 8-20 atoms, arylheterocyclyl including 8-20 atoms, heteroarylheterocyclyl including 8-20 atoms, arylheterocycloalkenyl including 8-20 atoms, heteroarylheterocycloalkenyl including 8-20 atoms, each of which is:
(i) substituted with from 1-5 R⁸, and
(ii) optionally substituted with from 1-4 R^{e}; wherein:
R⁸ is WA, wherein:
W at each occurrence is, independently, a bond; —O—; —S(O)ₜ—, wherein t is 0-2; —NR⁹—, wherein R⁹ is hydrogen or C₁-C₆ alkyl; C₁₋₆ alkylene; C₂₋₆ alkenylene; C₂₋₆ alkynylene; C₃₋₆ cycloalkylene; —W¹(C₁₋₆ alkylene)-; or —(C₁₋₆ alkylene)W¹—;
W¹ at each occurrence is, independently, —O—; —S(O)ₜ—, wherein t is 0-2; or —NR⁹—, wherein R⁹ is hydrogen or C₁-C₆ alkyl; and
A at each occurrence is, independently, C₆-C₁₈ aryl, heteroaryl including 5-16 atoms, C₈-C₂₀ arylcycloalkyl, heteroarylcycloalkyl including 8-20 atoms, C₈-C₂₀ arylcycloalkenyl, heteroarylcycloalkenyl including 8-20 atoms, arylheterocyclyl including 8-20 atoms, heteroarylheterocyclyl including 8-20 atoms, arylheterocycloalkenyl including 8-20 atoms, heteroarylheterocycloalkenyl including 8-20 atoms, each of which is:
(i) substituted with from 1-5 R¹⁰, and
(ii) optionally substituted with from 1-10 R^{e}; wherein:
R¹⁰ at each occurrence is, independently:
(i) —W²—C(O)OR¹¹; or
(ii) 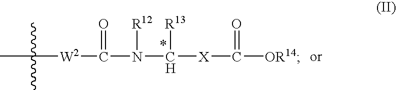
(iii) —W²—C(O)NR¹⁵R¹⁶; wherein:
W² at each occurrence is, independently, a bond; C₁₋₆ alkylene optionally substituted with from 1-3 R^{b}; C₂₋₆ alkenylene; C₂₋₆ alkynylene; C₃₋₆ cycloalkylene; or —W³(C₁₋₆ alkylene)-;
W³ at each occurrence is, independently, —O—; —S(O)ₜ—, wherein t is 0-2; or —NR⁹—, wherein R⁹ is hydrogen or C₁-C₆ alkyl;
each of R¹¹, R¹³, and R¹⁴ at each occurrence is, independently:
(i) hydrogen; or
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{c}; or
(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{b}; or
(v) C₆-C₁₈ aryl or heteroaryl including 5-16 atoms, each of which is optionally substituted with from 1-10 R^{d};
R¹² is hydrogen or C₁-C₆ alkyl;
X is a bond; C₁₋₆ alkylene optionally substituted with from 1-5 substituents selected from halo or R^{a}; C₂₋₆ alkenylene or C₂₋₆ alkynylene optionally substituted with from 1-5 R^{c}; or C₃₋₆ cycloalkylene optionally substituted with from 1-5 R^{b}; and
R¹⁵ and R¹⁶ together with the nitrogen atom to which each is attached is heterocyclyl including 3-10 atoms, which is:
(i) substituted with from 1-2 R^{f}; and
(ii) optionally substituted with from 1-4 R^{e};
each of R⁴, R⁵, R⁶, and R⁷ is, independently:
(i) hydrogen; or
(ii) R^{c}; or
(iii) C₁-C₂₀ alkyl or C₁-C₂₀ haloalkyl, each of which is optionally substituted with from 1-10 R^{a}; or
(iv) C₂-C₂₀ alkenyl or C₂-C₂₀ alkynyl, each of which is optionally substituted with from 1-10 R^{c}; or
(iv) C₇-C₂₀ aralkyl or heteroaralkyl including 6-20 atoms, each of which is optionally substituted with from 1-10 R^{b};
R^{a} at each occurrence is, independently:
(i) NR^{g}R^{h}; nitro; azido; hydroxy; oxo; thioxo; ═NRⁱ; C₁-C₂₀ alkoxy or C₁-C₂₀ haloalkoxy, each of which is optionally substituted with from 1-10 R^{a′}; C₆-C₁₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{b}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{b}; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h}); or
(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{b};
R^{a′} at each occurrence is, independently, NR^{g}R^{h}; nitro; azido; hydroxy; oxo; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; —P(O)(OR^{g})(OR^{h}); C₃-C₂₀ cycloalkyl, C₃-C₂₀ cycloalkenyl, heterocyclyl including 3-20 atoms, or heterocycloalkenyl including 3-20 atoms;
R^{b} at each occurrence is, independently:
(i) halo; NR^{g}R^{h}; nitro; azido; hydroxy; oxo; thioxo; ═NRⁱ; C₁-C₂₀ alkoxy or C₁-C₂₀ haloalkoxy, each of which is optionally substituted with from 1-10 R^{a}; C₆-C₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{b′}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{b′}; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h}); or
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{c}; or
(iv) C₆-C₁₈ aryl or heteroaryl including 5-16 atoms, each of which is optionally substituted with from 1-10 R^{d}; or
(v) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{b′};
R^{b′} at each occurrence is, independently, R^{a′}; halo; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d}; C₁-C₂₀ alkyl or C₁-C₂₀ haloalkyl, each of which is optionally substituted with from 1-10 R^{a}; C₂-C₂₀ alkenyl; C₂-C₂₀ alkynyl; or C₆-C₁₈ aryl or heteroaryl including 5-16 atoms, each of which is optionally substituted with from 1-10 R^{d};
R^{c} at each occurrence is, independently:
(i) halo; NR^{g}R^{h}; nitro; azido; hydroxy; oxo; thioxo; ═NRⁱ; C₁-C₂₀ alkoxy or C₁-C₂₀ haloalkoxy, each of which is optionally substituted with from 1-10 R^{a}; C₆-C₈ aryloxy or heteroaryloxy including 5-16 atoms, each of which is optionally substituted with from 1-10 R^{d}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{b}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{b}; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h}); or
(ii) C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{b}; or
(iii) C₆-C₁₈ aryl or heteroaryl including 5-16 atoms, each of which is optionally substituted with from 1-10 R^{d};
R^{d} at each occurrence is, independently:
(i) halo; NR^{g}R^{h}; nitro; azido; hydroxy; C₁-C₂₀ alkoxy or C₁-C₂₀ haloalkoxy, each of which is optionally substituted with from 1-10 R^{a}; C₆-C₁₈ aryloxy or heteroaryloxy including 5-16 atoms, each of which is optionally substituted with from 1-10 R^{d′}; C₇-C₂₀ aralkoxy, heteroaralkoxy including 6-20 atoms, C₃-C₁₆ cycloalkoxy, C₃-C₂₀ cycloalkenyloxy, heterocyclyloxy including 3-20 atoms, or heterocycloalkenyloxy including 3-20 atoms, each of which is optionally substituted with from 1-10 R^{b}; mercapto; C₁-C₂₀ thioalkoxy; C₁-C₂₀ thiohaloalkoxy; C₆-C₁₈ thioaryloxy or thioheteroaryloxy including 5-16 atoms, each of which is optionally substituted with from 1-10 R^{d′}; C₇-C₂₀ thioaralkoxy, thioheteroaralkoxy including 6-20 atoms, C₃-C₁₆ thiocycloalkoxy, C₃-C₂₀ thiocycloalkenyloxy, thioheterocyclyloxy including 3-20 atoms, or thioheterocycloalkenyloxy including 3-20 atoms, each of which is optionally substituted with from 1-10 R^{b}; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR^{g})(OR^{h});
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{c}; or
(iv) C₇-C₂₀ aralkyl, heteroaralkyl including 6-20 atoms, C₃-C₂₀ cycloalkyl, C₃-C₂₀ cycloalkenyl, heterocyclyl including 3-20 atoms, or heterocycloalkenyl including 3-20 atoms, each of which is optionally substituted with from 1-10 R^{b}; or
(v) C₆-C₁₈ aryl or heteroaryl including 5-16 atoms, each of which is optionally substituted with from 1-10 R^{d′};
R^{d′} at each occurrence is, independently, halo; NR^{g}R^{h}; nitro; azido; hydroxy; C₁-C₂₀ alkyl, C₁-C₂₀ haloalkyl, C₂-C₂₀ alkenyl; C₂-C₂₀ alkynyl; C₃-C₂₀ cycloalkyl; C₃-C₂₀ cycloalkenyl, heterocyclyl including 3-20 atoms; heterocycloalkenyl including 3-20 atoms; C₇-C₂₀ aralkyl; heteroaralkyl including 6-20 atoms; C₁-C₂₀ alkoxy; C₁-C₂₀ haloalkoxy; C₆-C₁₈ aryloxy; heteroaryloxy; C₇-C₂₀ aralkoxy; heteroaralkoxy including 6-20 atoms; C₃-C₁₆ cycloalkoxy; C₃-C₂₀ cycloalkenyloxy; heterocyclyloxy including 3-20 atoms; heterocycloalkenyloxy including 3-20 atoms; mercapto; C₁-C₂₀ thioalkoxy; C₁-C₂₀ thiohaloalkoxy; C₆-C₁₈ thioaryloxy; thioheteroaryloxy including 5-16 atoms; C₇-C₂₀ thioaralkoxy, thioheteroaralkoxy including 6-20 atoms, C₃-C₁₆ thiocycloalkoxy C₃-C₂₀ thiocycloalkenyloxy, thioheterocyclyloxy including 3-20 atoms, or thioheterocycloalkenyloxy including 3-20 atoms; cyano; —C(O)R^{j}, —C(O)OR^{j}; —OC(O)R^{j}; —C(O)SR^{j}; —SC(O)R^{j}; —C(S)SR^{j}; —SC(S)R^{j}; —C(O)NR^{g}R^{h}; —NR^{k}C(O)R^{j}; —C(NRⁱ)R^{j}; —OC(O)NR^{g}R^{h}; —NR^{k}C(O)NR^{g}R^{h}; —NR^{k}C(O)OR^{j}; —S(O)ₙR^{m}, wherein n is 1 or 2; —NR^{k}S(O)ₙR^{m}; or —P(O)(OR⁹)(OR^{h});
R^{e} at each occurrence is, independently, C₁-C₆ alkyl, optionally substituted with from 1-3 R^{a}; C₁-C₆ haloalkyl; mercapto; C₁-C₆ thioalkoxy optionally substituted with from 1-3 R^{a}; C₆-C₁₀ aryl or C₆-C₁₀ aryloxy, each of which is optionally substituted with from 1-10 R^{d}; halo; hydroxyl; NR^{g}R^{h}; nitro; C₂-C₆ alkenyl; C₂-C₆ alkynyl; C₁-C₆ alkoxy; C₁-C₆ haloalkoxy; cyano; —C(O)OR^{j}; or —C(O)R^{j};
R^{f} at each occurrence is, independently, —X—C(O)OR¹⁴, wherein each of X and R¹⁴ at each occurrence is, independently, as defined above;
each of R^{g}, R^{h}, Rⁱ, and R^{k}, at each occurrence is, independently:
(i) hydrogen; or
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{c}; or
(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{b}; or
(v) C₆-C₁₈ aryl or heteroaryl including 5-16 atoms, each of which is optionally substituted with from 1-10 R^{d}; or
(vi) —C(O)R^{j}, —C(O)OR^{j}; or —S(O)ₙR^{m};
R¹ at each occurrence is, independently:
(i) hydrogen; or
(ii) C₁-C₂₀ alkyl or C₁-C₂₀ haloalkyl, each of which is optionally substituted with from 1-10 R^{a}; or
(iii) C₂-C₂₀ alkenyl or C₂-C₂₀ alkynyl, each of which is optionally substituted with from 1-10 R^{c}; or
(iv) C₃-C₂₀ cycloalkyl, C₃-C₂₀ cycloalkenyl, heterocyclyl including 3-20 atoms, or heterocycloalkenyl including 3-20 atoms, C₇-C₂₀ aralkyl, or heteroaralkyl including 6-20 atoms, each of which is optionally substituted with from 1-10 R^{b}; or
(v) C₆-C₁₈ aryl or heteroaryl including 5-16 atoms, each of which is optionally substituted with from 1-10 R^{d}; and
R^{m} at each occurrence is, independently, R^{j}, OR^{j}, or NR^{g}R^{h};
or an N-oxide and/or a pharmaceutically acceptable salt thereof.

2. The compound of claim 1, wherein R¹ is hydrogen.

3. The compound of claim 1, wherein R² is C₁-C₆ alkyl, optionally substituted with from 1-2 R^{a}.

4. The compound of claim 3, wherein R² is CH₃.

5. The compound of claim 1, wherein R² is C₇-C₁₀ aralkyl, optionally substituted with from 1-3 R^{b}.

6. The compound of claim 5, wherein R² is benzyl.

7. The compound of claim 1, wherein R² is hydrogen.

8. The compound of claim 1, wherein R³ is phenyl, which is (a) substituted with 1 R⁵; and (b) optionally substituted with from 1-2 R^{e}.

9. The compound of claim 8, wherein R³ has formula (A-2):

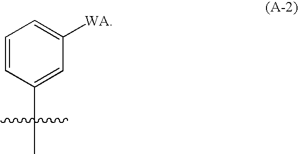

10. The compound of claim 1, wherein R³ is heteroaryl including 5-10 atoms, which is (a) substituted with 1 R⁸; and (b) optionally substituted with from 1-2 R^{e}.

11. The compound of claim 10, wherein R³ is pyridyl, thienyl, or indolyl, each of which is substituted with 1 R⁸.

12. The compound of claim 1, wherein W is —(C₁₋₆ alkylene)W¹—.

13. The compound of claim 12, wherein W¹ is —NR⁹—.

14. The compound of claim 13, wherein W is —CH₂NH—.

15. The compound of claim 12, wherein W¹ is —O—.

16. The compound of claim 15, wherein W is —CH₂O—.

17. The compound of claim 1, wherein A is C₆-C₁₀ aryl, which is (a) substituted with 1 R¹⁰; and (b) optionally substituted with from 1-6 R^{e}.

18. The compound of claim 17, wherein A has formula (B-1): 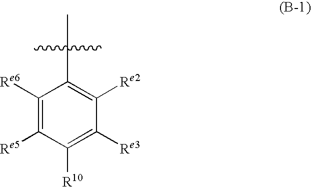
wherein:
each of R^{e2}, R^{e3}, R^{e5}, and R^{e6} is, independently, hydrogen or R^{e}.

19. The compound of claim 17, wherein A has formula (B-2): 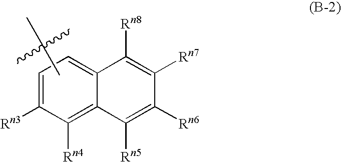
wherein:
each of Rⁿ³ and Rⁿ⁴ is, independently, hydrogen or R^{e}; and one of Rⁿ⁵, Rⁿ⁶, Rⁿ⁷, and Rⁿ⁸ is R¹⁰, and the others are each hydrogen or R^{e}.

20. The compound of claim 1, wherein R¹⁰ is —W²—C(O)OR¹¹.

21. The compound of claim 20, wherein R¹¹ is hydrogen.

22. The compound of claim 20, wherein W² is C₁-C₃ alkylene.

23. The compound of claim 20, wherein W² is CH₂ or a bond.

24. The compound of claim 1, wherein each of R⁴, R⁵ and R⁶ is hydrogen.

25. The compound of claim 1, wherein R⁷ is C₁-C₆ haloalkyl.

26. The compound of claim 25, wherein R⁷ is CF₃.

27. The compound of claim 1, wherein:
R² is:
(i) hydrogen; or
(ii) C₁-C₁₂ alkyl which is optionally substituted with from 1-5 R^{a}; or
(iii) C₇-C₂₀ aralkyl, which is optionally substituted with from 1-10 R^{b};
R³ is C₆-C₁₈ aryl or heteroaryl including 5-16 atoms, each of which is: (i) substituted with 1 R⁸, and (ii) optionally substituted with from 1-4 R^{e};
W is —(C₁₋₆ alkylene)W¹—;
W¹ at each occurrence is, independently, —O— or —NR⁹—, wherein R⁹ is hydrogen or C₁-C₃ alkyl; and
A at each occurrence is, independently, C₆-C₁₈ aryl, heteroaryl including 5-16 atoms, or C₈-C₂₀ arylcycloalkenyl, each of which is: (i) substituted with 1 R¹⁰, and (ii) optionally substituted with from 1-4 R^{e};
R¹⁰ is —W²—C(O)OR¹¹, and
W² is a bond or C₁₋₆ alkylene optionally substituted with from 1-3 R^{b}.

28. The compound of claim 27, wherein each of R¹, R⁴, R⁵, and R⁶ is hydrogen, and R⁷ is C₁-C₄ haloalkyl.

29. The compound of claim 28, wherein R² is CH₃, benzyl, or hydrogen.

30. The compound of claim 28, wherein R³ has formula (A-1): 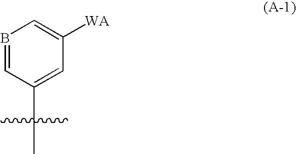
wherein B is CH or N.

31. The compound of claim 30, wherein W is —CH₂NH— or —CH₂O—.

32. The compound of claim 28, wherein R¹⁰ is —W²—C(O)OH, and W² is a bond or C₁₋₃ alkylene.

33. The compound of claim 28, wherein A has formula (B-1): 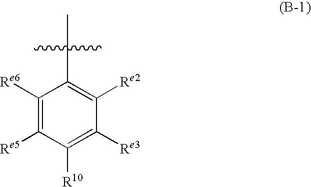
wherein:
each of R^{e2}, R^{e3}, R^{e5}, and R^{e6} is, independently, hydrogen, or R^{e}.

34. The compound of claim 33, wherein two of R^{e2}, R^{e3}, R^{e5}, and R^{e6} are CH₃, and the others are hydrogen, R¹⁰ is —W²—C(O)OH, and W² is CH₂ or a bond.

35. The compound of claim 28, wherein A has formula (B-3): 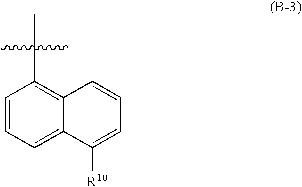
wherein R¹⁰ is —CH₂—C(O)OH.

36. The compound of claim 28, wherein R⁷ is CF₃.

37. The compound of claim 1, wherein the compound is selected from:
[5-({3-[8-(trifluoromethyl)quinolin-4-yl]benzyl}amino)-1-naphthyl]acetic acid;
{4-[({5-[3-benzyl-8-(trifluoromethyl)quinolin-4-yl]pyridin-3-yl}methyl)amino]-2,5-dimethylphenyl}acetic acid;
{5-[({5-[3-benzyl-8-(trifluoromethyl)quinolin-4-yl]pyridin-3-yl}methyl)amino]-1-naphthyl}acetic acid;
[2,5-dimethyl-4-({3-[8-(trifluoromethyl)quinolin-4-yl]benzyl}amino)phenyl]acetic acid;
[5-({3-[8-(trifluoromethyl)quinolin-4-yl]benzyl}oxy)-1-naphthyl]acetic acid;
6-[({5-[3-benzyl-8-(trifluoromethyl)quinolin-4-yl]pyridin-3-yl}methyl)amino]-2-naphthoic acid;
[2,5-dimethyl-4-({3-[3-methyl-8-(trifluoromethyl)quinolin-4-yl]benzyl}amino)phenyl]acetic acid;
[5-({3-[3-benzyl-8-(trifluoromethyl)quinolin-4-yl]benzyl}amino)-1-naphthyl]acetic acid;
5-({3-[3-methyl-8-(trifluoromethyl)quinolin-4-yl]benzyl}oxy)-1-naphthyl]acetic acid;
{4-[({5-[3-benzyl-8-(trifluoromethyl)quinolin-4-yl]pyridin-3-yl}methyl)amino]-2,3-dimethylphenyl}acetic acid;
[5-({3-[3-methyl-8-(trifluoromethyl)quinolin-4-yl]benzyl}amino)-1-naphthyl]acetic acid;
4-[({5-[3-benzyl-8-(trifluoromethyl)quinolin-4-yl]pyridin-3-yl}methyl)amino]-2,5-dimethylbenzoic acid;
6-({3-[3-methyl-8-(trifluoromethyl)quinolin-4-yl]benzyl}amino)-2-naphthoic acid;
[2,3-dimethyl-4-({3-[3-methyl-8-(trifluoromethyl)quinolin-4-yl]benzyl}amino)phenyl]acetic acid;
[4-({5-[3-benzyl-8-(trifluoromethyl)quinolin-4-yl]pyridin-3-yl}methoxy)-2,5-dimethylphenyl]acetic acid;
4-({3-[3-benzyl-8-(trifluoromethyl)quinolin-4-yl]benzyl}amino)-5-chloro-2-methoxybenzoic acid;
[5-({3-[3-benzyl-8-(trifluoromethyl)quinolin-4-yl]benzyl}oxy)-3,4-dihydronaphthalen-1-yl]acetic acid;
[4-({3-[3-benzyl-8-(trifluoromethyl)quinolin-4-yl]benzyl}amino)-2,3-dimethylphenyl]acetic acid;
[5-({3-[3-benzyl-8-(trifluoromethyl)quinolin-4-yl]benzyl}oxy)-1-naphthyl]acetic acid;
[2,3-dimethyl-4-({3-[8-(trifluoromethyl)quinolin-4-yl]benzyl}amino)phenyl]acetic acid;
6-({3-[3-benzyl-8-(trifluoromethyl)quinolin-4-yl]benzyl}amino)-2-naphthoic acid;
4-({3-[3-benzyl-8-(trifluoromethyl)quinolin-4-yl]benzyl}amino)-2,5-dimethylbenzoic acid;
[4-({3-[3-benzyl-8-(trifluoromethyl)quinolin-4-yl]benzyl}amino)-2,5-dimethylphenyl]acetic acid;
2,5-dimethyl-4-({3-[3-methyl-8-(trifluoromethyl)quinolin-4-yl]benzyl}amino)benzoic acid;
4-({3-[3-benzyl-8-(trifluoromethyl)quinolin-4-yl]benzyl}oxy)-3,5-dimethylbenzoic acid; and
3-[4-({3-[3-benzyl-8-(trifluoromethyl)quinolin-4-yl]benzyl}amino)phenyl]propanoic acid;
or a pharmaceutically acceptable salt thereof.

38. The compound of claim 1, wherein the compound is selected from:
N-[4-({3-[3-benzyl-8-(trifluoromethyl)quinolin-4-yl]phenoxy}methyl)benzoyl]methionine;
4-({3-[3-benzyl-8-(trifluoromethyl)quinolin-4-yl]phenoxy}methyl)-2-methoxybenzoic acid;
[4-({3-[3-benzyl-8-(trifluoromethyl)quinolin-4-yl]benzyl}amino)-1-naphthyl]acetic acid;
[3-({3-[3-benzyl-8-(trifluoromethyl)quinolin-4-yl]benzyl}amino)phenyl]acetic acid;
5-[({5-[3-benzyl-8-(trifluoromethyl)quinolin-4-yl]pyridin-3-yl}methyl)amino]-1-naphthoic acid;
{4-[({2-[3-benzyl-8-(trifluoromethyl)quinolin-4-yl]-3-thienyl}methyl)amino]-2,5-dimethylphenyl}acetic acid;
2-({3-[3-benzyl-8-(trifluoromethyl)quinolin-4-yl]benzyl}amino)-4-methylbenzoic acid;
[4-({3-[3-benzyl-8-(trifluoromethyl)quinolin-4-yl]benzyl}oxy)-2,5-dimethylphenyl]acetic acid;
[2-({3-[3-benzyl-8-(trifluoromethyl)quinolin-4-yl]benzyl}amino)-3,4-dimethylphenyl]acetic acid;
{4-[({5-[3-benzyl-8-(trifluoromethyl)quinolin-4-yl]pyridin-3-yl}methyl)amino]phenyl}acetic acid;
[5-({3-[3-benzyl-8-(trifluoromethyl)quinolin-4-yl]benzyl}oxy)-1,2,3,4-tetrahydronaphthalen-1-yl]acetic acid;
[4-({3-[3-benzyl-8-(trifluoromethyl)quinolin-4-yl]benzyl}oxy)-2,3-dihydro-1H-inden-1-yl]acetic acid;
6-({3-[3-benzyl-8-(trifluoromethyl)quinolin-4-yl]benzyl}oxy)-2-naphthoic acid;
1-[3-({3-[3-benzyl-8-(trifluoromethyl)quinolin-4-yl]phenoxy}methyl)benzoyl]piperidine-4-carboxylic acid;
2-({3-[3-benzyl-8-(trifluoromethyl)quinolin-4-yl]benzyl}amino)-4-fluorobenzoic acid;
[4-({3-[3-benzyl-8-(trifluoromethyl)quinolin-4-yl]benzyl}amino)phenyl]acetic acid;
3-[3-({3-[3-benzyl-8-(trifluoromethyl)quinolin-4-yl]benzyl}amino)phenyl]propanoic acid;
7-({3-[3-benzyl-8-(trifluoromethyl)quinolin-4-yl]benzyl}amino)-1-methyl-1H-indole-3-carboxylic acid;
4-({3-[3-benzyl-8-(trifluoromethyl)quinolin-4-yl]benzyl}oxy)-3-methoxybenzoic acid;
6-({3-[8-(trifluoromethyl)quinolin-4-yl]benzyl}amino)-2-naphthoic acid;
{4-[({5-[3-benzyl-8-(trifluoromethyl)quinolin-4-yl]-2-thienyl}methyl)amino]-2,5-dimethylphenyl}acetic acid;
5-({3-[8-(trifluoromethyl)quinolin-4-yl]benzyl}amino)-1-naphthoic acid;
2-({3-[3-benzyl-8-(trifluoromethyl)quinolin-4-yl]benzyl}amino)-6-(trifluoromethyl)benzoic acid;
[4-({3-[3-benzyl-8-(trifluoromethyl)quinolin-4-yl]benzyl}amino)-2-chlorophenyl]acetic acid;
3-({3-[3-benzyl-8-(trifluoromethyl)quinolin-4-yl]benzyl}amino)benzoic acid;
3-({3-[3-benzyl-8-(trifluoromethyl)quinolin-4-yl]benzyl}amino)-4-fluorobenzoic acid;
4-({3-[3-benzyl-8-(trifluoromethyl)quinolin-4-yl]benzyl}amino)-3-methylbenzoic acid;
1-[4-({3-[3-benzyl-8-(trifluoromethyl)quinolin-4-yl]phenoxy}methyl)benzoyl]praline;
2-({3-[3-benzyl-8-(trifluoromethyl)quinolin-4-yl]benzyl}amino)-6-methoxybenzoic acid;
5-({3-[3-benzyl-8-(trifluoromethyl)quinolin-4-yl]benzyl}amino)-1-naphthoic acid;
[6-({3-[3-benzyl-8-(trifluoromethyl)quinolin-4-yl]benzyl}oxy)-1-naphthyl]acetic acid; and
2-({3-[3-benzyl-8-(trifluoromethyl)quinolin-4-yl]benzyl}amino)-6-fluorobenzoic acid;
or a pharmaceutically acceptable salt thereof.

39. The compound of claim 1, wherein the compound is selected from:
1-[3-({3-[3-benzyl-8-(trifluoromethyl)quinolin-4-yl]phenoxy}methyl)benzoyl]praline;
1-[4-({3-[3-benzyl-8-(trifluoromethyl)quinolin-4-yl]phenoxy}methyl)benzoyl]piperidine-4-carboxylic acid;
N-[4-({3-[3-benzyl-8-(trifluoromethyl)quinolin-4-yl]phenoxy}methyl)benzoyl]histidine;
[3-({3-[3-benzyl-8-(trifluoromethyl)quinolin-4-yl]benzyl}oxy)-4-methylphenyl]acetic acid;
5-({3-[3-benzyl-8-(trifluoromethyl)quinolin-4-yl]benzyl}amino)-1-methyl-1H-indole-2-carboxylic acid;
{4-[({4-[3-benzyl-8-(trifluoromethyl)quinolin-4-yl]-3-thienyl}methyl)amino]-2,5-dimethylphenyl}acetic acid;
[4-({3-[8-(trifluoromethyl)quinolin-4-yl]benzyl}amino)-1-naphthyl]acetic acid;
2-[5-({3-[8-(trifluoromethyl)quinolin-4-yl]benzyl}oxy)-1-naphthyl]pent-4-ynoic acid;
4-({3-[3-benzyl-8-(trifluoromethyl)quinolin-4-yl]benzyl}oxy)-3,5-dichlorobenzoic acid;
[6-({3-[8-(trifluoromethyl)quinolin-4-yl]benzyl}oxy)-1-naphthyl]acetic acid;
[5-({3-[3-benzyl-8-(trifluoromethyl)quinolin-4-yl]benzyl}oxy)-2-naphthyl]acetic acid;
{4-[({4-[3-benzyl-8-(trifluoromethyl)quinolin-4-yl]-2-thienyl}methyl)amino]-2,5-dimethylphenyl}acetic acid;
2-({3-[3-benzyl-8-(trifluoromethyl)quinolin-4-yl]benzyl}amino)-5-methylbenzoic acid;
N-[3-({3-[3-benzyl-8-(trifluoromethyl)quinolin-4-yl]phenoxy}methyl)benzoyl]histidine;
2-chloro-4-({3-[3-methyl-8-(trifluoromethyl)quinolin-4-yl]benzyl}amino)benzoic acid;
[4-({3-[3-benzyl-8-(trifluoromethyl)quinolin-4-yl]phenoxy}methyl)-2,5-dimethylphenyl]acetic acid;
4-({3-[3-benzyl-8-(trifluoromethyl)quinolin-4-yl]benzyl}amino)-2-methylbenzoic acid;
N-[3-({3-[3-benzyl-8-(trifluoromethyl)quinolin-4-yl]phenoxy}methyl)benzoyl]methionine;
7-({3-[3-benzyl-8-(trifluoromethyl)quinolin-4-yl]benzyl}amino)-1H-indole-2-carboxylic acid;
3-({3-[3-benzyl-8-(trifluoromethyl)quinolin-4-yl]benzyl}amino)-2-naphthoic acid;
5-({3-[3-benzyl-8-(trifluoromethyl)quinolin-4-yl]benzyl}amino)-1-methyl-1H-indole-3-carboxylic acid;
5-({3-[3-methyl-8-(trifluoromethyl)quinolin-4-yl]benzyl}amino)-1-naphthoic acid;
N-[4-({3-[3-benzyl-8-(trifluoromethyl)quinolin-4-yl]phenoxy}methyl)benzoyl]leucine;
5-({3-[3-benzyl-8-(trifluoromethyl)quinolin-4-yl]benzyl}amino)-2-chlorobenzoic acid;
7-({3-[3-benzyl-8-(trifluoromethyl)quinolin-4-yl]benzyl}amino)-1H-indole-3-carboxylic acid;
4-({3-[3-benzyl-8-(trifluoromethyl)quinolin-4-yl]benzyl}oxy)-3-fluorobenzoic acid;
4-({3-[3-benzyl-8-(trifluoromethyl)quinolin-4-yl]benzyl}oxy)-3-chlorobenzoic acid;
[7-({3-[3-benzyl-8-(trifluoromethyl)quinolin-4-yl]benzyl}oxy)-1-naphthyl]acetic acid;
4-({3-[3-benzyl-8-(trifluoromethyl)quinolin-4-yl]benzyl}amino)-2-chlorobenzoic acid;
[3,4-dimethyl-5-({3-[3-methyl-8-(trifluoromethyl)quinolin-4-yl]benzyl}amino)phenyl]acetic acid;
2-({3-[3-benzyl-8-(trifluoromethyl)quinolin-4-yl]benzyl}oxy)-6-fluorobenzoic acid;
3-({3-[3-benzyl-8-(trifluoromethyl)quinolin-4-yl]phenoxy}methyl)benzoic acid;
2-({3-[3-benzyl-8-(trifluoromethyl)quinolin-4-yl]benzyl}amino)-4-chlorobenzoic acid;
3-({3-[3-benzyl-8-(trifluoromethyl)quinolin-4-yl]benzyl}oxy)-4-fluorobenzoic acid; and
2-({3-[3-benzyl-8-(trifluoromethyl)quinolin-4-yl]benzyl}amino)-6-methylbenzoic acid;
or a pharmaceutically acceptable salt thereof.

40. The compound of claim 1, wherein the compound is selected from:
3-({3-[3-benzyl-8-(trifluoromethyl)quinolin-4-yl]benzyl}amino)-4-methylbenzoic acid;
1-({3-[3-benzyl-8-(trifluoromethyl)quinolin-4-yl]benzyl}oxy)-2-naphthoic acid;
N-[3-({3-[3-benzyl-8-(trifluoromethyl)quinolin-4-yl]phenoxy}methyl)benzoyl]leucine;
2-({3-[3-benzyl-8-(trifluoromethyl)quinolin-4-yl]benzyl}amino)benzoic acid;
3-({3-[3-benzyl-8-(trifluoromethyl)quinolin-4-yl]benzyl}oxy)-4-methoxybenzoic acid;
5-({3-[3-benzyl-8-(trifluoromethyl)quinolin-4-yl]benzyl}amino)-1H-indole-3-carboxylic acid;
4-({3-[3-benzyl-8-(trifluoromethyl)quinolin-4-yl]phenoxy}methyl)benzoic acid;
4-({3-[3-benzyl-8-(trifluoromethyl)quinolin-4-yl]benzyl}amino)benzoic acid;
2-({3-[3-benzyl-8-(trifluoromethyl)quinolin-4-yl]benzyl}oxy)-5-methylbenzoic acid;
4-({3-[3-benzyl-8-(trifluoromethyl)quinolin-4-yl]benzyl}amino)-2,3-dimethylbenzoic acid;
2-({3-[3-benzyl-8-(trifluoromethyl)quinolin-4-yl]benzyl}oxy)-5-fluorobenzoic acid;
7-({3-[3-benzyl-8-(trifluoromethyl)quinolin-4-yl]benzyl}amino)-1-methyl-1H-indole-2-carboxylic acid;
4-fluoro-2-({3-[3-methyl-8-(trifluoromethyl)quinolin-4-yl]benzyl}amino)benzoic acid;
[7-({3-[8-(trifluoromethyl)quinolin-4-yl]benzyl}oxy)-1-naphthyl]acetic acid;
4-({3-[3-benzyl-8-(trifluoromethyl)quinolin-4-yl]benzyl}amino)-5-bromo-2,3-dimethylbenzoic acid;
3-({3-[3-benzyl-8-(trifluoromethyl)quinolin-4-yl]benzyl}oxy)-2-naphthoic acid;
4-({3-[3-benzyl-8-(trifluoromethyl)quinolin-4-yl]benzyl}amino)-3-(trifluoromethoxy)benzoic acid;
2-({3-[3-benzyl-8-(trifluoromethyl)quinolin-4-yl]benzyl}amino)-6-chlorobenzoic acid;
2-({3-[3-benzyl-8-(trifluoromethyl)quinolin-4-yl]benzyl}amino)-5-fluorobenzoic acid;
4-({3-[3-benzyl-8-(trifluoromethyl)quinolin-4-yl]phenoxy}methyl)-3-chlorobenzoic acid;
[8-({3-[3-benzyl-8-(trifluoromethyl)quinolin-4-yl]benzyl}oxy)-2-naphthyl]acetic acid;
2-fluoro-6-({3-[3-methyl-8-(trifluoromethyl)quinolin-4-yl]benzyl}amino)benzoic acid;
{5-[({5-[8-(trifluoromethyl)quinolin-4-yl]pyridin-3-yl}methyl)amino]-1-naphthyl}acetic acid;
{2,5-dimethyl-4-[({5-[3-methyl-8-(trifluoromethyl)quinolin-4-yl]pyridin-3-yl}methyl)amino]phenyl}acetic acid;
[5-({5-[8-(trifluoromethyl)quinolin-4-yl]pyridin-3-yl}methoxy)-1-naphthyl]acetic acid;
{5-[({5-[3-methyl-8-(trifluoromethyl)quinolin-4-yl]pyridin-3-yl}methyl)amino]-1-naphthyl}acetic acid;
{4-[({5-[8-(trifluoromethyl)quinolin-4-yl]pyridin-3-yl}methyl)amino]phenyl}acetic acid;
5-{3-[3-benzyl-8-(trifluoromethyl)quinolin-4-yl]phenoxy}pyrazine-2-carboxylic acid;
3-[3-(3-benzyl-8-chloroquinolin-4-yl)phenoxy]-5-bromobenzoic acid;
3-[3-(3-benzyl-8-chloroquinolin-4-yl)phenoxy]-5-fluorobenzoic acid;
4-{3-[3-phenyl-8-(trifluoromethyl)quinolin-4-yl]phenoxy}benzoic acid;
3-[3-(3-benzyl-8-chloroquinolin-4-yl)phenoxy]benzoic acid;
3-{3-[3-benzyl-8-(trifluoromethyl)quinolin-4-yl]phenoxy}benzoic acid;
4-({4-[3-benzyl-8-(trifluoromethyl)quinolin-4-yl]-1H-indol-1-yl}methyl)benzoic acid;
4-({3-[3-benzyl-8-(trifluoromethyl)quinolin-4-yl]phenyl}ethynyl)-3-methylbenzoic acid;
3-({3-[3-benzyl-8-(trifluoromethyl)quinolin-4-yl]phenyl}ethynyl)-4-methylbenzoic acid;
3′-[3-benzyl-8-(trifluoromethyl)quinolin-4-yl]biphenyl-3-carboxylic acid; and
3-({3-[3-benzyl-8-(trifluoromethyl)quinolin-4-yl]phenyl}ethynyl)-4-methoxybenzoic acid;
or a pharmaceutically acceptable salt thereof.

41. A pharmaceutical composition comprising a compound of formula (I) as claimed in claim 1, or a pharmaceutically acceptable salt thereof, and a pharmaceutically acceptable carrier.

42. A method of preventing or treating a Liver X receptor-mediated disease or disorder, the method comprising administering to a subject in need of such treatment an effective amount of a compound of formula (I) as claimed in claim 1 or a pharmaceutically acceptable salt thereof.

43. A method of preventing or treating a cardiovascular disease, the method comprising administering to a subject in need of such treatment an effective amount of a compound of formula (I) as claimed in claim 1 or a pharmaceutically acceptable salt thereof.

44. A method of preventing or treating Alzheimer's disease, the method comprising administering to a subject in need of such treatment an effective amount of a compound of formula (I) as claimed in claim 1 or a pharmaceutically acceptable salt thereof.

45. A method of preventing or treating type I or type II diabetes, the method comprising administering to a subject in need of such treatment an effective amount of a compound of formula (I) as claimed in claim 1 or a pharmaceutically acceptable salt thereof.

46. A method of preventing or treating an inflammatory disease, the method comprising administering to a subject in need of such treatment an effective amount of a compound of formula (I) as claimed in claim 1 or a pharmaceutically acceptable salt thereof.

47. A method of treating a connective tissue disease, the method comprising administering to a mammal in need thereof an effective amount of a compound of formula (I) as claimed in claim 1 or a pharmaceutically acceptable salt thereof.

48. A method of treating skin aging, the method comprising administering to a mammal in need thereof an effective amount of a compound of formula (I) as claimed in claim 1 or a pharmaceutically acceptable salt thereof.


SEQUENCE LISTING (9 sequences)

SEQ ID NO: 1 - DNA, 25 bp, Mus musculus
gaagccagttgtgcaaaactaaatt

SEQ ID NO: 2 - DNA, 20 bp, Mus musculus
gcaacactgtggtggcttca

SEQ ID NO: 3 - DNA, 24 bp, Mus musculus
cacatctcatctcccgacccagca

SEQ ID NO: 4 - DNA, 21 bp, Mus musculus
agccatgtacgtagccatcca

SEQ ID NO: 5 - DNA, 21 bp, Mus musculus
tctccggagtccatcacaatg

SEQ ID NO: 6 - DNA, 28 bp, Mus musculus
tgtccctgtatgcctctggtcgtaccac

SEQ ID NO: 7 - DNA, 22 bp, Homo sapiens
caacatgaatgccattttccaa

SEQ ID NO: 8 - DNA, 21 bp, Homo sapiens
ataatcccctgaacccaagga

SEQ ID NO: 9 - DNA, 25 bp, Homo sapiens
taaagccatgccctctgcaggaaca